(19) United States
(12) Patent Application Publication
(10) Pub. No.: US 2008/0213404 A1
(43) Pub. Date: Sep. 4, 2008

(54) Hif Modulating Compounds and Methods of Use Thereof

(76) Inventors: Randall S. Johnson, San Diego, CA (US); Carole Peyssonnaux, Antony (FR); Victor Nizet, San Diego, CA (US); Emmanuel Theodorakis, San Diego, CA (US)

Correspondence Address: SCHWEGMAN, LUNDBERG & WOESSNER, P.A., P.O. BOX 2938, MINNEAPOLIS, MN, 55402, US

(21) Appl. No.: 11/883,692

(22) PCT Filed: Feb. 3, 2006
(86) PCT No.: PCT/US06/04004

§ 371 Date: Aug. 3, 2007

Related U.S. Application Data
(60) Provisional application No. 60/649,544, filed on Feb. 4, 2005.

(51) Int. Cl. A61K 36/00; C12Q 1/20

(52) U.S. Cl. 424/725; 435/29

(57) ABSTRACT

A method is provided for treating subjects, including humans, with infection or virulence by pathogens. The method involves administering an agent in amounts effective to eradicate or reduce infections and/or an inflammatory response caused by pathogens. Methods for identifying compounds useful as anti-infectives that decrease the immune resistance, virulence, or growth of microbes are also provided. More particularly, there are provided methods for identifying compounds which increase accumulation or stability or activity, or alternatively decrease the degradation of HIF-1a protein.

### STATEMENT OF GOVERNMENT INTEREST

This invention was made partially with U.S. Government support from the United States Department of Health and Human Services, National Institutes of Health, under Grant No. CA 82515. The U.S. Government has certain rights in the invention.

### BACKGROUND

The eradication of invading microorganisms depends initially on innate immune mechanisms that preexist in all individuals and act within minutes of infection. Phagocytic cell types, including macrophages and neutrophils, play a key role in innate immunity because they can recognize, ingest, and destroy many pathogens without the aid of an adaptive immune response. The effectiveness of myeloid cells in innate defense reflects their capacity to function in low oxygen environments. Whereas in healthy tissues oxygen tension is generally 20-70 mm HG (i.e. 2.5-9% oxygen), much lower levels (<1% oxygen) have been described in wounds and necrotic tissue foci (Arnold et al., Br J Exp Pathol 68, 569 (1987); Vogelberg & Konig, Clin Investig 71, 466 (1993); Negus et al., Am J Pathol 150, 1723 (1997)).

The adaptive response of mammalian cells to the stress of oxygen depletion is coordinated by the action of hypoxia-inducible transcription factor 1 (HIF-1). HIF-1 is a heterodimer whose expression is regulated by oxygen at the protein level. The protein stability of the α-subunit (HIF-1α) is regulated by a family of prolyl hydroxylases. This process is directed by the interaction of HIF-1α with the von Hippel-Lindau tumor-suppressor protein (vHL). Under hypoxia, prolyl hydroxylase activity is inhibited, and HIF-1α accumulates and translocates into the nucleus, where it binds to HIF-1β, constitutively expressed. The heterodimer HIF-1 binds to the hypoxic response elements (HREs) of target gene regulatory sequences, resulting in the transcription of genes implicated in the control of metabolism and angiogenesis as well as apoptosis and cellular stress (4). Some of these direct target genes include glucose transporters, glycolytic enzymes, erythropoietin, and the angiogenic factor VEGF. Two additional HIF subunits have subsequently been cloned and named HIF-2 (5-7) and HIF-3 (8), but their regulation is less well understood.

Hypoxia-inducible factor (HIF-1) is an oxygen-dependent transcriptional activator, which plays crucial roles in the angiogenesis of tumors and mammalian development. HIF-1 consists of a constitutively expressed HIF-1β subunit and one of three subunits (HIF-1α, HIF-2α or HIF-3α). The stability and activity of HIF-1α are regulated by various post-translational modifications, hydroxylation, acetylation, and phosphorylation. Therefore, HIF-1α interacts with several protein factors including PHD, pvHL, ARD-1, and p300/CBP. Under normoxia, the HIF-1α subunit is rapidly degraded via the von Hippel-Lindau tumor suppressor gene product (vHL)-mediated ubiquitin-proteasome pathway. The association of vHL and HIF-1α under normoxic conditions is triggered by the hydroxylation of prolines and the acetylation of lysine within a polypeptide segment known as the oxygen-dependent degradation (ODD) domain. On the contrary, in the hypoxia condition, HIF-1α subunit becomes stable and interacts with coactivators such as p300/CBP to modulate its transcriptional activity. Eventually, HIF-1 acts as a master regulator of numerous hypoxia-inducible genes under hypoxic conditions. The heterodimer HIF-1 binds to the hypoxic response elements (HREs) of target gene regulatory sequences, resulting in the transcription of genes implicated in the control of cell proliferation/survival, glucose/iron metabolism and angiogenesis, as well as apoptosis and cellular stress. Some of these direct target genes include glucose transporters, the glycolytic enzymes, erythropoietin, and angiogenic factor vascular endothelial growth factor (VEGF). Moreover, it was reported that the activation of HIF-1α is closely associated with a variety of tumors and oncogenic pathways. Hence, the blocking of HIF-1α itself or certain HIF-1α interacting proteins inhibit tumor growth. Based on these findings, HIF-1 has been a prime target for anticancer therapies.

HIF-prolyl hydroxylase (HIFPH or HPH) has also been a target for modulating HIF activity. Companies such as Fibrogen (www.fibrogen.com, 2005) have been actively researching HIF activity (HIF1, HIF2 and HIF3) to develop inhibitors of prolyl hydroxylases (HPH1, HPH2 and HPH3, respectively) to direct HIF-mediated protective mechanisms such as erythropoiesis and cytoprotection; for the treatment of anemia and acute renal failure, respectively; for the treatment of cardiovascular and neural ischemia; in treating metabolic disorders such as obesity, and directing certain aspects of HIF-mediated vascular biology for enhanced wound healing and chronic ischemic disease applications (see, e.g., PCT Application Nos. WO 03/049686, WO 03/053997, WO 04/052284, WO 04/052285, WO 04/108121, WO 04/108681, WO 05/007192, WO 05/011696, WO 05/034929, and U.S. Provisional Application No. 2004/254215, each incorporated herein by reference). This has lead to identifying more selective compounds to modulate specific HIF activity as HIF isoforms have been associated with different activities, e.g., the HIF2 isoform is responsible for EPO induction, whereas the HIF1 isoform is required for vasculogenesis, a distinction with relevance to the design of selective HIF-stabilizing agents (e.g., selective erythropoietic compounds for anemia therapy).

Recently, employing conditional gene targeting in the myeloid cell lineage, HIF-L a control of the metabolic shift to glycolysis was shown to be essential for myeloid cell-mediated inflammatory responses (Cramer et al., Cell 112, 645 (2003)). Employing mice with conditional knockouts of HIF-1α and vHL, this study provided evidence that deletion of HIF-1α impaired an inflammatory response. However, it did not suggest an inflammatory response could be regulated by manipulating the activity of HIF-1α. In addition, this study discusses preliminary in vitro evidence that HIF-α-deficient macrophages may impair Group B Streptococcus (GBS) bactericidal activity, although it did not suggest a proper innate immune response to bacterial infection could be coordinated.

Macrophages are one population of effector cells involved in immune responses. Their role in natural immunity includes mediation of phagocytosis, as well as release of cytokines and cytotoxic mediators. They also facilitate the development of acquired immunity through antigen presentation and release of immunomodulatory cytokines. Although macrophages are immune effectors, they are also susceptible to infection by agents such as bacteria, protozoa, parasites, and viruses (The Macrophage, C. E. Lewis & J.O'D. McGee. eds., IRL Press at Oxford University Press, New York, N.Y., 1992). Viruses capable of infecting macrophages include several RNA viruses such as measles virus (MV) (e.g., Joseph et al., J. Virol. 16, 1638-1649, 1975), respiratory syncytial virus (RSV) (Midulla et al., Am. Rev. Respir. Dis. 140, 771-777, 1989), and human immunodeficiency virus type 1 (HIV-1) (Meltzer and Gendelman, in Macrophage Biology and Activation, S. W. Russell and S. Gordon, eds., Springer-Verlag, New York, N.Y., pp. 239-263(1992: Potts et al., Virology 175, 465-476, 1990).

### SUMMARY OF THE INVENTION

The present invention reveals a completely novel and unique approach to treatment of infection by enhancing the innate immune system in eukaryotic host subjects.

In a first aspect, the invention provides methods of modulating the activity of at least one HIF-1 protein. In general, the methods comprise contacting at least one HIF-1 protein or HIF-1 interacting protein with a substance that modulates the activity of the HIF-1 protein, or causing contact between the protein and substance. The methods can be in vitro methods, in vivo methods, or comprise both in vitro and in vivo steps. In further embodiments, the method is a method of treating a subject infected or at risk of infection by a microbial pathogen, and comprises administering to a subject a therapeutically effective amount of a substance that increases the amount or activity of HIF-1. In embodiments, the method is a method of killing microbial pathogens, such as bacterial and viral pathogens. In embodiments, the method is a method of increasing the microbial pathogen-killing activity of immune cells. Without limiting the present invention to any particular mechanism, the substance can increase the activity of the HIF-1 protein by acting directly or indirectly on the HIF-1 protein to stabilize the protein, protect it from inhibition, or to increase the activity of the protein. Alternatively, the substance can increase the activity of the HIF-1 protein by inhibiting or otherwise blocking the activity of compounds that inhibit the activity of the HIF-1 protein. In certain embodiments, the method includes introducing into at least one cell of the subject, such as immune cells, a nucleic acid that encodes at least one HIF-1 protein, and permitting the cell to express the HIF-1 protein. In a preferred embodiment, the method is a method of improving the treatment of microbial infections by administering a substance that increases the activity or level of at least one HIF-1 protein in a subject suffering from the microbial infection or at increased risk of microbial infection.

In another aspect, the invention provides methods of identifying substances that modulate the activity of at least one HIF-1 protein. In general, the method comprises providing at least one HIF-1 protein, contacting the protein with at least one substance suspected of having the ability to modulate the activity of a HIF-1 protein, and determining whether the substance modulates the activity of the HIF-1 protein. The methods can be in vivo methods, in vitro methods, or a combination of in vivo and in vitro steps. The method of this aspect of the invention can be a method of identifying substances that modulate the response of a subject to an infection by a microbial pathogen. In view of the method of this aspect of the invention, it is evident that the invention includes the use of a HIF-1 protein to identify substances that modulate its activity, including use to identify pharmaceutically active compounds such as drugs or prodrugs.

In a third aspect, the invention provides substances that modulate the activity of at least one HIF-1 protein. The substances can modulate the activity of the HIF-1 protein in vitro, in vivo, or both. The substances can increase the activity or amount of HIF-1 protein in a composition, such as one comprising an immune cell, or decrease the activity or amount. The mode of action of the substance can be direct on the HIF-1 protein or indirect, for example by binding to an inhibitor of the HIF-1 protein or by enhancing expression of nucleic acids encoding the HIF-1 protein.

Accordingly, in preferred embodiments, the invention relates to approaches to treatment of infection by increasing the killing capacity of the cells of the innate immune system of a subject, particularly to pathogens. In one embodiment, the present invention is directed to methods and compounds for treating infection or virulence by modulating the activity and/or level of HIF-1, particularly HIF-1α. In an alternative embodiment, the invention relates to screening procedures which identify compounds for inhibiting infection or disease in a eukaryotic host organism, or which induce or stimulate a host's pathogenic defense mechanisms. The invention also relates to these compounds and the use of such compounds as anti-pathogens.

The hypoxia-responsive transcription factor HIF-1α is essential for regulation of inflammation in vivo. We have found that bacterial infection induces HIF-1α expression in myeloid cells even under normoxic conditions, and show that HIF-1α regulates the generation of critical molecular effectors of immune defense including granule proteases, antimicrobial peptides, nitric oxide, and TNF-α. Bacterial infection induced a subset of HIF-1α target genes specifically related to microbial killing, demonstrating that HIF-1α has an essential function in innate immunity distinct from hypoxic response. We show herein that HIF-1α function is critical for myeloid cell bactericidal activity and the ability of the host to limit systemic spread of infection from an initial tissue focus. Increased activity of the HIF-1α pathway through vHL deletion supported myeloid cell production of defense factors and improved bactericidal capacity. Pharmacologic inducers of HIF-1α can also boost bacterial killing and NO production in a HIF-1α-specific fashion, and thus represent a novel mechanism for enhancing innate immune responses to bacterial infection.

### BRIEF DESCRIPTION OF THE DRAWINGS

FIG. 1 is a series of graphs showing Bacteria increase HIF-1 protein expression and stimulate HIF-1 transcriptional activity. (A and B) Macrophages were incubated under hypoxia (0.1%) or with GAS, MRSA, S. typhimurium (ST), or P. aeruginosa (PA) at an MOI equal to 5-10 under normoxic conditions for 4 hours. Expression of HIF-1 was normalized to β-actin levels and quantified with ImageQuantTL software (Amersham Biosciences). (C) HRE-luciferase BM-derived macrophages were incubated either with GAS or heat-inactivated GAS at an MOI equal to 5-10 under hypoxia (1%) or with the addition of mimosine (800 μM), desferrioxamine mesylate (150 μM), or CoCl₂ (150 μM) for 18 hours. Statistical analyses were performed using unpaired Student's t test. **P<0.01; ***P<0.001.

FIG. 2 is a series of graphs showing HIF-1 regulates bactericidal activity of myeloid cells. (A) Intracellular killing of GAS by WT, HIF-1-null, or vHL-null macrophages. BM-derived macrophages were inoculated with GAS at an MOI equal to 2.5 and cultured under normoxic (white bars) or hypoxic (0.1%; black bars) conditions for 1 hour after antibiotic treatment. Statistical analyses were performed using unpaired Student's t test. *P<0.05; **P<0.01. (B) Loss of HIF-1 in macrophages decreases intracellular killing of GAS and of P. aeruginosa. WT (black bars) or HIF-1-null (white bars) BM-derived macrophages were incubated with bacteria for 1 hour before antibiotics were added. Intracellular killing was analyzed by determination of viable CFUs in macrophage lysates at the specified time points after bacterial uptake. Experiments were performed in triplicate. SEM is displayed. Experiment shown is representative of 3 repeated studies. (C) Loss of vHL in BM-derived macrophages increases intracellular killing of GAS and of P. aeruginosa. Experiments were performed in triplicate and are representative of 3 repeated studies. SEM is displayed. (D) Pharmacologic agonists of HIF-1 increase myeloid cell bactericidal activity. Preincubation (5 hours) with desferrioxamine mesylate (DFO), CoCl₂, OH-pyridone, or Mim increased the intracellular killing capacity of WT macrophages against GAS. ***P<0.001.

FIG. 3 is a series of graphs which illustrate HIF-1 deletion renders mice more susceptible to GAS infection. (A) Area of necrotic ulcer and (B) loss of weight in individual WT (squares) and HIF-1 myeloid-null mice (triangles) 4 days after infection with GAS. (C) Representative appearance of GAS-induced necrotic skin ulcers in WT and HIF-1 myeloid-null mice. A total of 11 mice in each group were tested in 3 paired experiments. (D) Bacterial counts in the blood, spleen, and skin of WT and HIF-1 myeloid-null mice infected with GAS. The fold difference in quantitive GAS culture between WT and HIF-1-null animals is annotated. Statistical analyses were performed using unpaired Student's t test. *P<0.05; **P<0.01.

FIG. 4 is a series of images and graphs which illustrate HIF-1 is not critical for neutrophil endothelial transcytosis or oxidative burst function. (A) Hypoxia is present in lesions generated by GAS infection. Immunostaining for hypoxic markers in WT mouse skin upon GAS infection. Magnification, ×100 (top); ×200 (bottom). The control corresponds to the omission of primary antibody. (B) Similar numbers of neutrophils in WT and HIF-1-null mouse skin tissue observed by immunostaining at 6, 12, and 24 hours after infection. Magnification, ×100. (C) Migratory capacity of activated neutrophils across endothelium is not affected by the deletion of HIF-1. Count of neutrophils transcytosing pulmonary endothelial monolayer toward GAS or fMLP stimulus is shown. (D) HIF-1 activity does not affect oxidative burst capacity. Flow cytometry of leukocytes derived from WT (squares), HIF-1-null (triangles) and vHL-null (inverted triangles) mice. Oxidative burst capacity as measured by fluorescence before (0 seconds) and after the addition of a reagent designed to stimulate leukocyte phagocytic and oxidative activity as described in Methods. Data are representative of the results obtained for 4 individuals per genotype.

FIG. 5 provides graphs of Production of granule proteases and of murine CRAMP is regulated by HIF-1. NE (A) and cathepsin G (B) activity in WT, HIF-1-null, vHL-null and in a mix of WT and HIF−/− blood leukocytes. (C) Neutrophils were processed for immunoblotting with anti-CRAMP antibody (upper panels) or anti-β-actin antibody (lower panels). (D) HIF-1 regulates CRAMP at the mRNA level. Neutrophils were cultured under normoxic or hypoxic (0.1%) conditions. Total neutrophil RNA was extracted and mRNA polyA+ isolated by an Oligotex mRNA spin-column protocol (QIAGEN). WT neutrophils were arbitrarily set to 1 unit following normalization to β-actin RNA levels. Statistical analyses were performed using unpaired Student's t test. *P<0.05; **P<0.01; ***P<0.001.

FIG. 6 provides graphs showing HIF-1 and vHL regulate NO production. (A) Total RNA from WT, HIF-1−/−, and vHL−/− bone marrow-derived macrophages infected with GAS isolated 3 hours after antibiotic treatment. iNOS mRNA was quantified by RT-PCR. WT, nonstimulated macrophages were arbitrarily set to 1 unit following normalization to ribosomal RNA levels. (B) NO production under GAS stimulation ±1.5 mM AG (1-amino-2-hydroxyguanidine, p-toluenesulfate; Calbiochem). BM-derived macrophages were cultured for 20 hours, conditioned supernatant collected, and NO protein levels measured by the Griess assay. (C) Mim enhances iNOS expression of WT macrophages stimulated by GAS. Total RNA from WT and HIF-1-null BM-derived macrophages infected with GAS±Mim isolated 3 hours after antibiotic treatment. iNOS mRNA was quantified by RT-PCR. WT, noninfected macrophages were arbitrarily set to 1 unit following normalization to ribosomal RNA levels. Statistical analyses performed by unpaired Student's t test. **P<0.01; ***P<0.001. (D) Inhibition of iNOS by AG blunts observed differences between HIF-1-null and WT microbicidal activity. (E) Inhibition of iNOS prevents GAS-induced HIF-1 expression. Expression of HIF-1 is normalized to β-actin levels.

FIG. 7 is a series of graphs illustrating HIF-1 and vHL regulate TNF-production. (A) Total RNA from WT, HIF-1−/−, and vHL−/− bone marrow-derived macrophages infected with GAS were isolated 3 hours after antibiotic treatment. TNF-mRNA was quantified by RT-PCR. WT, nonstimulated macrophages were arbitrarily set to 1 unit following normalization to ribosomal RNA levels. (B) inhibition of iNOS decreases TNF-production. BM-derived macrophages were cultured for 1 hour after antibiotic treatment. Conditioned supernatant was harvested and TNF-protein analyzed by ELISA (eBiosciences). Statistical analyses were performed using unpaired Student's t test. ***P<0.001.

FIG. 8 depicts, without limiting the mechanism of the present invention, a model for the role of HIF-1 in myeloid cell innate immune function. Bactericidal mechanisms can be maintained in an “off” state while myeloid cells circulate in the oxygen-rich bloodstream. Transendothelial migration toward an infectious focus occurs in a HIF-1 independent fashion, but upon diapedesis, specific bactericidal mechanisms are activated through HIF-1 induction in response to the declining oxygen gradient. Further potent stimulation of the HIF-1 transcriptional pathway is provided after direct encounter with the infecting bacterial pathogen. HIF-1 regulates the generation of critical molecular effectors of immune defense, including granule proteases, antimicrobial peptides, and TNF-. HIF-1 also stimulates the production of NO, which not only acts as an antimicrobial agent and inflammatory mediator but further amplifies myeloid cell bactericidal activity via HIF-1 stabilization.

FIG. 9 shows a graph illustrating mortality was reduced significantly in HIF-1α knockout mice when eight week-old male WT and HIF-1α myeloid null mice were injected IP with 15 mg/kg of lipopolysaccharide (LPS, Sigma) or saline (control). Deletion of HIF in the myeloid lineage renders mice less susceptible to LPS-induced sepsis and mortality

FIG. 10 shows a graph illustrating significantly lower levels of TNFα were found in the HIF-1α one hour after LPS injection, the mice were bled for analysis of tumor necrosis factor alpha (TNFα) levels in the serum. TNFα is a proximal mediator of the sepsis cascade. Deletion of HIF in the myeloid lineage renders mice less susceptible to LPS induction of tumor necrosis factor-1α, an important mediator of septicemia.

FIG. 11 provides a graph illustrating severe hypothermia is a marker of lethal septicemia in mice. HIF−/− mice are also less hypothermic than WT during the course of LPS challenge. Deletion of HIF in the myeloid lineage renders mice less susceptible to LPS-induced sepsis and its associated hypthermia.

FIG. 12 provides a graph illustrating the induction of HIF by the agonist mimosine to increase the bactericidal capacity of freshly isolated human blood against the pathogen Staphylococcus aureus.

FIG. 13 provides a graph illustrating induction of HIF by the agonist mimosine to increase the bactericidal capacity of freshly isolated human neutrophils against the pathogen Staphylococcus aureus.

FIG. 14 provides a graph illustrating induction of HIF by the agonist mimosine to increase the bactericidal capacity of freshly isolated human neutrophils against vincomycin-resistant Enterococcus species.

FIG. 15 provides a graph illustrating induction of HIF by the agonist mimosine to increase the bactericidal capacity of cultured human monocytic cells against the pathogen Staphylococcus aureus.

FIG. 16 provides a graph illustrating induction of HIF by the agonist mimosine to increase the bactericidal capacity of cultured human monocytic cells against vincomycin-resistant Enterococcus species.

### DETAILED DESCRIPTION OF THE INVENTION

The present invention reveals a completely novel and unique approach to treatment of infection by enhancing the innate immune system in eukaryotic host subjects.

In a first aspect, the invention provides methods of modulating the activity of at least one HIF-1 protein. In general, the methods comprise contacting at least one HIF-1 protein or HIF-1 interacting protein with at least one substance that modulates the activity of HIF-1 protein, or causing contact between the protein and substance such that the substance modulates the activity of HIF-1 protein. The methods can be in vitro methods, in vivo methods, or comprise both in vitro and in vivo steps. The substance can be any substance that modulates the activity of a HIF-1 protein in vivo or in vitro, such as, but not limited to, a small organic or inorganic molecule, a polypeptide, a nucleic acid, or another organic macromolecule, such as a polysaccharide, lipopolysaccharide, or combination or complex of two or more of these.

In alternate embodiments, the method is a method of up-regulating a HIF-1 protein. Up-regulation can be anything that results in an increase in the amount or activity of a HIF-1 protein. Non-limiting examples of up-regulation are activation of the protein to increase transcription of HRE-controlled genes; enhancing nuclear translocation of the protein; improving the stability (e.g., half-life) of the protein; and improving the binding affinity or strength of the protein to DNA. Up-regulating also includes blocking or reducing deactivation of the HIF-1 protein, for example by hydroxylation and/or acetylation of the HIF-1 protein. Among other things, up-regulation further includes increasing the amount of HIF-1 protein in a sample under consideration, for example by increasing the amount expressed from a gene or by introducing multiple copies of a HIF-1 encoding sequence.

Thus, without limiting the present to any particular mechanism, with regard to an exemplary mode of action, the substance can increase the activity of the HIF-1 protein by acting directly or indirectly on the HIF-1 protein to stabilize the protein, protect it from inhibition, or to increase the activity of the protein. Alternatively, the substance can increase the activity of the HIF-1 protein by inhibiting or otherwise blocking the activity of compounds that inhibit the activity of the HIF-1 protein. In certain embodiments, the method includes introducing into at least one cell of the subject, such as immune cells, a nucleic acid that encodes at least one HIF-1 protein, and permitting the cell to express the HIF-1 protein.

Accordingly, there are provided methods of utilizing compounds which up-regulate HIF-1 protein to enhance the innate immune response to pathogens. A preferred compound of the invention is an agent that inhibits the activity of HIF prolyl hydroxylases, particularly HIF-1α prolyl hydroxylases, such as vHL, more preferably, compounds which stabilize HIF-1α.

In other embodiments, the method is a method of down-regulating a HIF-1 protein. Down-regulating can be anything that results in a decrease in the amount or activity of a HIF-1 protein. Down-regulation can be useful in making models of infection, in testing antibiotics for activity, in treating sepsis or inflammatory disorders, and various other things.

In further embodiments, the method of modulating the activity of a HIF-1 protein is a method of treating at least one cell to improve microbial killing by that cell. According to the invention, microbial killing includes, but is not necessarily limited to, killing of bacteria, fungi and viruses. Thus, this aspect of the method can be a method of killing bacteria. It likewise can be a method of reducing the number of bacteria in a sample, such as in a body of a subject. It further can be a method of reducing the number of viral particles in a sample. In certain embodiments, the method of treating is a method of enhancing the killing activity of immune cells. In yet other embodiments, the method is a method of enhancing the microbial-killing effect of an antibiotic.

Accordingly, the method of treating can be a method of treating a subject infected or at risk of infection by a microbial pathogen, and comprises administering to a subject a therapeutically effective amount of a substance that increases the amount or activity of HIF-1. In embodiments, the method is a method of killing microbial pathogens, such as bacterial, fungal and viral pathogens. The method can reduce the number of pathogens in the subject or can completely or essentially completely eliminate the pathogen from the subject.

In certain embodiments, the method of treating is a method of combination therapy with one or more antibiotics. In such embodiments, the method comprises administering an effective amount of a substance that activates at least one HIF-1 protein and administering an effective amount of at least one antibiotic. The amount of each to be administered can vary depending on the amount of the other administered. Thus, the methods can be methods of reducing the amount of antibiotic necessary to successfully treat a bacterial infection. It likewise can be a method of reducing the time required to successfully treat a bacterial infection. It is envisioned that a reduction in the amount of antibiotic administered and the amount of time for treatment will increase compliance with recommended dosing regimens, and will improve clinical outcomes of treatment regimens and reduce selection for antibiotic resistance.

In a preferred embodiment, the method is a method of improving the treatment of microbial infections by administering a substance that increases the activity or level of at least one HIF-1 protein in a subject suffering from the microbial infection.

In view of the above methods, it is evident that the present invention provides for the use of HIF-1 to treat microbial infections. In preferred embodiments, increase in the activity or level of at least one HIF-1 protein is used to achieve the results discussed herein.

The various embodiments of this aspect of the invention, as well as others, are discussed in more detail below.

In another aspect, the invention provides methods of identifying substances that modulate the activity of at least one HIF-1 protein. In general, the method comprises providing at least one HIF-1 protein, contacting the protein with at least one substance suspected of having the ability to modulate the activity of a HIF-1 protein, and determining whether the substance modulates the activity of the HIF-1 protein. The methods can be in vivo methods, in vitro methods, or a combination of in vivo and in vitro steps. The substance can be in any form and in any composition. For example, the compound can be a pure compound of known chemical composition and quantity. Alternatively, it can be, for example, an unknown substance present in a mixture of numerous other substances. Thus, the method can include contacting at least one HIF-1 protein with a sample that contains or is suspected of, but not known to, contain a substance that can modulate the activity of at least one HIF-1 protein. The methods can identify substances that increase the amount or activity of a HIF-1 protein, or can identify substances that decrease the amount or activity of a HIF-1 protein. Accordingly, the method can be a method of identifying drugs, prodrugs, lead compounds, or candidates for drugs or prodrugs.

The methods can be practiced to screen one substance at a time or can be practiced to screen multiple substances at one time. Accordingly, in certain embodiments, the method is a method of high-throughput screening of substances for HIF-1 modulating activity.

The method of this aspect of the invention can be a method of identifying substances that modulate the response of a subject to an infection by a microbial pathogen. For example, the method can be a method of identifying substances that improve the in vivo effectiveness of antibiotics. Improvement can be in the amount needed to successfully treat a microbial infection, or the amount of time needed for successful treatment.

In view of the method of this aspect of the invention, it is evident that the invention includes the use of a HIF-1 protein to identify substances that modulate its activity, including use to identify pharmaceutically active compounds such as drugs or prodrugs. The HIF-1 protein can be used as a purified or semi-purified protein, or can be used as it exists in one or more cells (the cell being either a cell in which the protein is naturally produced or a recombinant cell producing the HIF-1 protein heterologously).

These embodiments and others are described in more detail below.

In a third aspect, the invention provides substances that modulate the activity of at least one HIF-1 protein. The substances can modulate the activity of the HIF-1 protein in vitro, in vivo, or both. The substances can increase the activity or amount of HIF-1 protein in a composition, such as one comprising an immune cell, or decrease the activity or amount. The mode of action of the substance can be direct on the HIF-1 protein or indirect, for example by binding to an inhibitor of the HIF-1 protein or by enhancing expression of nucleic acids encoding the HIF-1 protein. Numerous substances can be identified through practice of the present invention, including, but not limited to, cathepsin G and nitric oxide. The substance can be in any physical form (solid, liquid, gas) and in any composition (e.g., a purified solution or powder; a pharmaceutical composition comprising the substance and at least one other component, such as a pharmaceutically acceptable carrier or excipient; a composition comprising the substance and another biologically active or inactive component; and the like).

In view of the present disclosure, it is evident that the present invention provides for the use of a substance to treat microbial infections in a subject, where the substance modulates, and preferably increases, the amount or activity of at least one HIF-1 protein in at least one cell of the subject. Preferably, the cell is an immune cell. Accordingly, the invention provides for the use of a substance that modulates the amount or activity of at least one HIF-1 protein to improve the clinical response of a subject infected with a microbial pathogen. In embodiments, the substance reduces the amount of antibiotic needed to successfully treat the subject. In embodiments, the substance reduces the amount of time needed to successfully treat the subject.

Accordingly, in preferred embodiments, the invention relates to approaches to treatment of infection by increasing the killing capacity of the cells of the innate immune system of a subject, particularly to pathogens. In one embodiment, the present invention is directed to methods and compounds for treating infection or virulence by modulating the activity and/or level of HIF-1, particularly HIF-1α. In an alternate embodiment, the invention relates to screening procedures which identify compounds for inhibiting infection or disease in a eukaryotic host organism, or which induce or stimulate a host's pathogenic defense mechanisms. The invention also relates to these compounds and the use of such compounds as anti-pathogens.

Various embodiments of the invention will now be described in more detail with reference to the Figures and data.

As employed herein, the term “transcription factor” includes HIF-1 proteins that are involved in gene regulation in both prokaryotic and eukaryotic organisms. The term “HIF-1”, as used herein, includes both the heterodimer complex and the subunits thereof, HIF-1α and HIF-1. The HIF 1 heterodimer consists of two helix-loop-helix proteins; these are termed HIF-1α, which is the oxygen-responsive component (see, e.g., accession no. Q16665 (and the homologues thereof), U.S. Pat. No. 6,562,799, U.S. Provisional Application No. 2003/0176349 and WO 96/39426A1), and HIF-1β. The latter is also known as the aryl hydrocarbon receptor nuclear translocator (ARNT). In addition are included homologues, analogues, and isoforms of HIF-1, particularly HIF-L a. Preferably, the term refers to the human form of HIF-1α (see, e.g., Accession No. NM001530/Q9NWT60/U22431/AB073325/AF208487/AF304431), although also contemplated are Accession Nos. Q9XTA5/AB018398/BAA78675 (bovine), AF057308/O35800/CAA70701 (rat), AF003695/AAC52730/AFO57308/Q61221 (mouse), AY713478 (squirrel); Q9YIB9 (avian), Q98SW2 (amphibian), AY971808 (antelope); Xenopus laevis HIF-1α (Genbank Accession No. CAB96628), Drosophila melanogaster HIF-1α(Genbank Accession No. JC485 1), AY326951 (zebrafish) and chicken HIF-1α(Genbank Accession No. ABA02179/BAA34234) and the like. Others species of interest would be dogs, cats, and other domesticated and farm animals, such as pigs and horses. HIF-1α may also be any mammalian or non-mammalian protein or fragment thereof. HIF-1α gene sequences may also be obtained by routine cloning techniques, for example by using all or part of a HIF-1α gene sequence described above as a probe to recover and determine the sequence of a HIF-1α gene in another species. A fragment of HIF-1α of interest is any fragment retaining at least one functional or structural characteristic of HIF-1α. Fragments of HIF-1α include, e.g. the regions defined by huma HIF-1α from amino acids 401 to 603 (Huang et al., (1998) PNAS, USA. 95:7987-7992), amino acid 531 to 575: (Jiang et al. (1997) J Biol. Chem. 272:19253-19260), amino acid 556 to 575 (Tanimoto et al., (2000) EMBO. J. 19:4298-4309), amino acid 557 to 571 (Srinivas et al. (1999) Biochem Biophys Res. Commun 260:557-561), and amino acid 556 to 575 (Ivan and Kaelin (2001) Science 292:464-468). Further, HIF-1αfragments include any fragment containing at least one occurrence of the motif LXXLAP, e.g. as occurs in the human HIF-1α native sequence at L₃₉₇TLLAP and L₅₅₉EMLAP.

Other HIFs of interest are huma HIF-2α (Genbank Accession No. AAB41495) and HIF-3α (Genbank Accession No. AAD22668/AB118749); murine HIF-2α (Genbank Accession No. BAA20130 and AAB41496) and HIF-3α (Genbank Accession No. AAC72734); rat HIF-2α (Genbank Accession No. CAB96612) and HIF-3α (Genbank Accession No. CAB96611), and the like.

The term “HIF-1 interacting protein” includes the von Hippel-Lindau tumor suppressor protein (vHL, Hon et al., Nature 417:975-8 (2002); Min et al., Science 296:1886-9 (2002) vHL and other hydroxylases (also referred herein as HIF hydroxylases such as the prolyl hydroxylases HPH-1/PHD-3, HPH-2/PHD-2 and HPH-3/PHD-1 (Huang et al., J Biol Chem 277:39792-800 (2002); Metzen et al., J Cell Sci 116:1319-26 (2003)), dehydroxylases, ubiquitylation and deubiquitylation enzymes, ARD1 acetyltransferase (Jeong et al., Cell 111:709-20 (2002), factor inhibiting HIF-1 (F1H-1, Hewitson et al., (2002); Lando et al., Genes Dev 16:1466-71 (2002); PCT Application Nos. WO03028663, WO04035812, WO02074981); inhibitory PAS domain protein (IPAS, Makino et al., Nature 414:550-4 (2002), and the like, which interact with one or more proteins comprising the HIF-1 heterodimer and/or modulate the activity thereof. Of particular interest are huma HIF-1 interacting proteins (see, e.g., Accession Nos. P40337, NP 000542, NP937799, NP005154, NP060372, NP003363, and the like) and homologues, analogues and isoforms thereof (including animal homologues). Those of skill in the art will readily be able to identify additional HIF-1 interacting proteins suitable in the present invention. HIF PH(HPH) includes members of the Egl-Nine (EGLN) gene family described by Taylor (2001, Gene 275:125-132), and characterized by Aravind and Koonin (2001, Genome Biol 2:RESEARCH0007), Epstein et al. (2001, Cell 107:43-54), and ruick and McKnight (2001, Science 294: 1337-1340). Examples of HIF prolyl hydroxylase enzymes include human SM-20 (EGLN1) (GenBank Accession No. AAG33965; Dupuy et al. (2000) Genomics 69:348-54), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511; Taylor, supra), mouse EGLN1 (GenBank Accession No. CAC425 15), EGLN2 (GenBank Accession No. CAC42511), and EGLN3 (SM-20) (GenBank Accession No. CAC42517); and rat SM-20 (GenBank Accession No. AAA19321). Additionally, HIF PH may include Caenorhabditis elegans EGL-9 (GenBank Accession No. AAD56365) and Drosophila melanogaster CG1114 gene product (GenBank Accession No. AAF52050). HIF prolyl hydroxylase also includes any fragment of the foregoing full-length proteins that retain at least one structural or functional characteristic.

The term “interact” includes close contact between molecules that results in a measurable effect, e.g., the binding of one molecule to another. For example, a transcription factor can interact with a transcription factor responsive element and alter the level of transcription of DNA. Likewise, compounds can interact with a transcription factor and alter the activity of that transcription factor.

The language “transcription factor responsive element” includes a nucleic acid sequence which can interact with a transcription factor (e.g., promoters or enhancers or operators) which are involved in initiating transcription. Transcription factor responsive elements responsive to various transcription factors are known in the art and additional responsive elements can be identified by one of ordinary skill in the art.

The term “hypoxia responsive element” includes a nucleic acid sequence which can interact with the HIF-1 heterodimer, e.g., promoters or enhancers which are involved in regulating transcription of a nucleic acid sequence, such as the hypoxia response elements (HRE, Dachs et al., Nat Med 3:515-20 (1997); Lemmon et al., Gene Ther 4:791-6 (1997)).

The term “subject” includes plants and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans) which are capable of suffering from a microbial disorder. The term “subject” also comprises immunocompromised subjects, who may be at a higher risk for infection, such as AIDS patients. Additionally, “subject” may also be animals and plants that there may be a desire to immunocompromise, such as rodents that are immune to pathogenic infections. “Subject” may also be a cell, a population of cells, a tissue, an organ, or an organism, preferably to human and constituents thereof.

As employed herein, the term “innate immune response” refers to the immune response of subjects to innately prevent, eradicate or reduce pathogenic infections by granulocytes, leukocytes, monocytes, macrophages, mononuclear phagocytes, neutrophils, and the like. The effectiveness of such agents, particularly neutrophils and macrophages, in innate antibacterial defense reflects a diverse array of highly specialized cellular functions, including phagocytic uptake of the bacterium, generation of phagolysosomes, production of reactive oxygen species, activation of inducible nitric oxide synthase (iNOS), and release of antimicrobial peptides (e.g. cathelicidins, defensins) and granule proteases (e.g. elastase, cathepsin).

The terms “treating”, and “treatment” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease, i.e., infection. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The invention is directed towards treating a patient's suffering from disease related to pathological infection. The term “prophylaxis” are used herein to refer to a measure or measures taken for the prevention or partial prevention of a disease or condition.

As used herein, the term “pathogen” includes both obligate and opportunistic organisms including bacteria, protozoa, fungi, nematodes, viruses, and other factors which may cause infective and/or inflammatory responses. In one embodiment, the invention is directed to treating infectivity or virulence of pathogens, particularity to antibiotic resistance, although resistance is not necessarily included in the terms “infectivity” or “virulence” as used herein. Accordingly, in one embodiment, the instant invention pertains to methods of reducing the infectivity or virulence of a pathogen. Preferably, as used herein, the term “infectivity or virulence” includes the ability of an organism to establish itself in a host by evading the host's barriers and immunologic defenses or cause disease.

Microbial pathogens such as bacteria, protozoa, fungi, nematodes, and viruses include a large and diverse group of organisms capable of infecting animals and plants. Initiation of an infection occurs when the infecting organism is pathogenic, and the host is susceptible to pathogenic invasion. After establishing contact with susceptible cells or tissues of the host, the pathogen acquires nutrients from its host, facilitating its own survival. During the infection process the pathogen activates a cascade of molecular, biochemical, and physiological processes, the result of which is the release of substances detrimental to the host and the development of disease (See, e.g., Scientific American Medicine, W.H. Freeman and Co., San Francisco, 1995; Agrios, G. N., Plant Pathology, Academic Press, 1988 Finlay B B, Falkow S. Common themes in microbial pathogenicity revisited. Microbiol Mol Biol Rev. 1997 June; 61(2):136-69.). The pathogenic effects of microbes are produced in a variety of ways.

As used herein the term “reporter gene” includes any gene which encodes an easily detectable product which is operably linked to a regulatory sequence, e.g., to a transcription factor responsive promoter. By operably linked it is meant that under appropriate conditions an RNA polymerase may bind to the promoter of the regulatory region and proceed to transcribe the nucleotide sequence such that the reporter gene is transcribed. In preferred embodiments, a reporter gene consists of the transcription factor responsive promoter linked in frame to the reporter gene. In certain embodiments, however, it may be desirable to include other sequences, e.g., transcriptional regulatory sequences, in the reporter gene construct. For example, modulation of the activity of the promoter may be effected by altering the RNA polymerase binding to the promoter region, or, alternatively, by interfering with initiation of transcription or elongation of the mRNA. Thus, sequences which are herein collectively referred to as transcriptional regulatory elements or sequences may also be included in the reporter gene construct. In addition, the construct may include sequences of nucleotides that alter translation of the resulting mRNA, thereby altering the amount of reporter gene product.

Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); PhoA, alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368), green and other similar fluorescent proteins (U.S. Pat. No. 5,491,084; WO96/23898) in their original and enhanced versions, and the like. Additional reporter genes include the endogenous genes activated by the HIF-1 heterodimer, including, but not limited to NO, granule proteases (cathepsin G, neutrophil elastase) and cathelicidin, glycolytic enzymes, glucose transporter (GLUT)-1, erythropoietin (EPO), and vascular endothelial growth factor (VEGF). (Jiang, et al., (1996) J. Biol. Chem., 271:17771-17778; Iliopoulus, et al., (1996) Proc. Natl. Acad. Sci. USA, 93:10595-10599; Maxwell, et al., (1999), Nature, 399:271-275; Sutter, et al., (2000) Proc. Natl. Acad. Sci. USA, 97:4748-4753; Cockman, et al., (2000) J. Biol. Those of skill in the art will readily be aware of reporter genes and proteins suitable for the present invention.

### Implications

Our studies have used conditional gene targeting in the myeloid cell lineage to demonstrate that HIF-1α transcriptional regulation plays an important role in innate immunity to infection. Activation of HIF-1α under hypoxia enhances microbicidal activity, and HIF-1α pathways are responsive to microbial stimulation even under normoxia. While certain myeloid cell functions including endothelial transmigration and respiratory burst activation appear independent of HIF-1α control, the present invention describes, without being limited to any particular mechanism, that the transcription factor HIF-1α is involved directly or indirectly in the regulation of specific immune functions including NO, granule proteases (cathepsin G, neutrophil elastase) and cathelicidin antimicrobial peptides. The marked reduction of granule protease and cathelicidin expression in HIF-1α-deficient neutrophils correlates to diminished microbicidal activity in vitro and failure to control infection in vivo, lending support to recent studies uncovering a key role for these neutrophil effectors in mammalian innate immunity (15, 24). The effectiveness of neutrophils and macrophages in innate antimicrobial defense reflects a diverse array of highly specialized cellular functions including phagocytic uptake of the microbe, production of reactive oxygen species, activation of iNOS, and release of antimicrobial peptides (e.g., cathelicidins, defensins) and granule proteases (e.g., elastase, cathepsin).

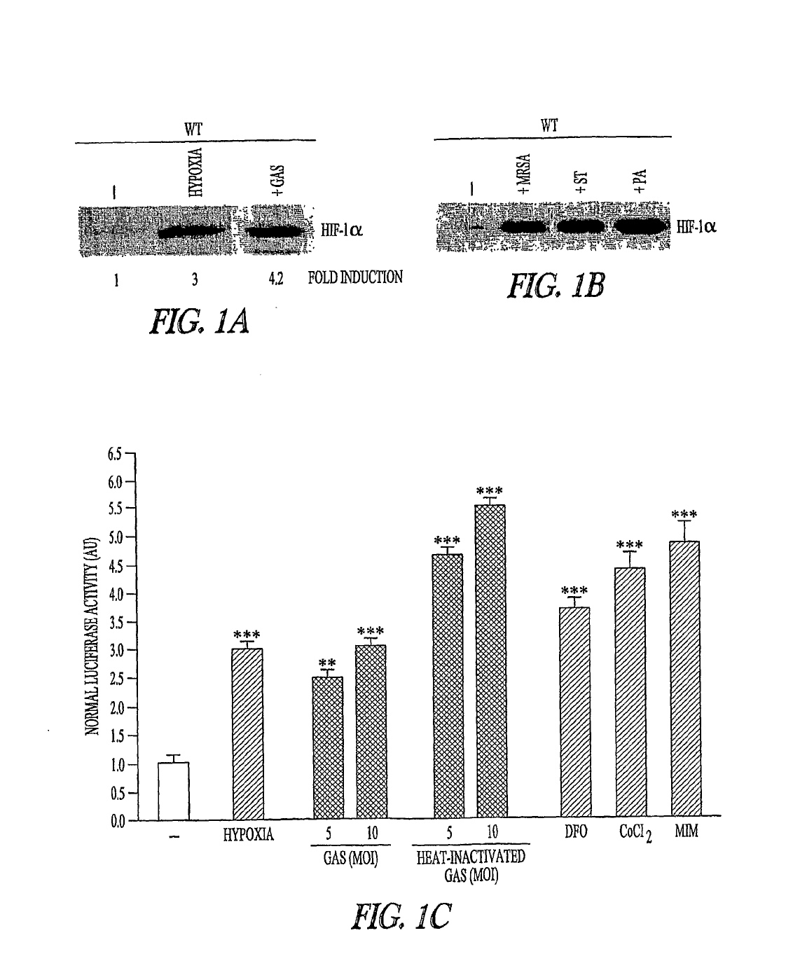

Successful control of infection in the peripheral tissues requires that host myeloid phagocytic cells function effectively in hypoxic environments. The challenge to immune defense is made more critical when the microbial toxins or local edema damage host cells and the vascular supply of oxygen to the tissues becomes further compromised. The placement of essential microbial killing functions of myeloid cells under regulation of HIF-1α therefore represents an elegant controlled-response system (FIG. 8). Microbicidal mechanisms can be maintained in an “off” state while the myeloid cells circulate in the oxygen-rich bloodstream, and then be activated in response to the declining oxygen gradient encountered upon diapedesis and entry into the infected tissues. Additional more potent stimulation of the HIF-1α transcriptional pathway is then provided by direct encounter with the microbe (FIG. 1A). A regulatory mechanism by which HIF-1α targets genes involved in microbial killing ensures that the corresponding inflammatory mediators are expressed preferentially in tissue foci of infection, but not in healthy tissues where inflammatory damage might otherwise harm host cells.

Our experiments also reveal that NO production is a myeloid cell killing mechanism principally regulated by HIF-1α during microbial infection. Further, we suggest that NO is likely to play a key role in the amplification of the inflammatory response through stimulation of TNF-α. Although the effects of inflammatory cytokines on regulating NO production have been extensively studied (38-40), the reverse relationship, pertaining to the effect of NO on cytokines, remains controversial (41-44). A recent study demonstrated that suppression of NO could inhibit LPS-induced TNF-α and interleukin-1 release, and pinpointed such modulation to the pretranslational level (45). We find here that macrophage production of TNF-α is dependent on NO levels controlled in turn by HIF-1α-transcriptional regulation of iNOS.

Recent data has established that HIF-1α is subjected to stability regulation by soluble intracellular messengers, such as NO and TNF-α (33, 34). With such processes at play, one can envision that HIF-1α is situated at the center of an amplification loop mechanism for innate immune activation: stimulation of HIF-1α by oxygen depletion and microbial exposure induces the production of NO and TNF-α, which function not only to generate inflammation and control bacterial proliferation, but also as regulatory molecules to further stabilize HIF-L a in myeloid cells recruited to the infectious focus.

The relative contributions of HIF-1 and HIF-2 to the regulation of gene expression in hypoxic macrophages is still under debate. Detectable levels of HIF-2α, but not HIF-1α, have been found in a human promonocytic cell line following hypoxic induction in vitro and in tumor-associated macrophages (46, 47). In contrast, immunoreactive HIF-1α has been detected in human macrophages in the hypoxic synovia of arthritic human joints (10), and human macrophages accumulate higher levels of HIF-1 than of HIF-2 when exposed to tumor-specific levels of hypoxia in vitro (9). Our present results also clearly support a specific and independent action of HIF-1α. These findings suggest that HIF-1 may be the major hypoxia-inducible transcription factor in macrophages.

In summary, our results demonstrate that HIF-1α not only helps myeloid cells shift to glycolytic metabolism (11) but also functions in coordinating a proper innate immune response for microbial killing. The in vivo studies confirm that the HIF-1α pathway can play a critical role in controlling proliferation of a pathogen in compromised tissues. Recent commentaries based on our work have suggested that downregulation of HIF-1α could have a therapeutic effect in disease states characterized by chronic inflammation (48, 49). We now have shown that medically important microbial species such as GAS, methicillin-resistant S. aureus (MRSA), P. aeruginosa, and Salmonella species can trigger HIF-1α expression. Thus, the present studies suggest the design and use of pharmaceutical HIF-1α agonists (or vHL antagonists) to boost myeloid cell microbicidal activity for a novel approach for adjunctive therapy of complicated infections due to antibiotic-resistant pathogens or compromised host immunity.

The methods of the invention provide a simple means for identifying and utilizing factors and compounds capable of either inhibiting pathogenicity or enhancing an organism's resistance capabilities to a pathogen. Accordingly, a chemical entity discovered to have medicinal or agricultural value using the methods described herein are useful as either drugs, protectants, or as information for structural modification of existing anti-pathogenic compounds, e.g., by rational drug design. Such methods are useful for screening compounds having an effect on a variety of pathogens including, but not limited to, bacteria, viruses, fungi, annelids, nematodes, platyhelminthes, and protozoans. Examples of pathogenic bacteria include, without limitation, Aerobacter, Aeromonas, Acinetobacter, Agrobacterium, Actinobacteria, Actinomycetales, Archaea, Azorhizobium, Bacillus, Bacteroides, Bartonella, Bortella, Brucella, Burkholderia, Calyinmatobacterium, Campylobacter, Caulobacter group, Citrobacter, Clostridium, Cornyebacterium, Crenarchaeota, Cyanobacteria, Edwardsiella, Enterococcus, Enterobacter, Escherichia, Euryarchaeota, Firinicutes, Francisella, Haemophilus, Hafnia, Helicobacter, Klebsiella, Kluyvera, Lactobacillus, Legionella, Listeria, Mesorhizobium, Methanococci; Methanosarcina, Methanosarcinales, Methanosarcinaceae, Morganella, Moraxella, Mycobacteria, Neisseria, Nostocales, Nostocaceae, Nostoc, Oxalobacter, Pectobacterium, Pediococcus, Photobacterium, Phyllobacteria, Proteus, Providencia, Proteobacteria, Pseudomonas, Ralstonia, Rhizobiaceae, Salmonella, Serratia, Shigella, Sinorhizobium, Staphylococcus, Streptonyces, Streptomycineae, Streptomyeetaceae, Streptococcus, Sulfolobales, Sulfolobaceae, Sulfolobus, Thermoprotei, Thermotoga, Thermotogae, Thermotogales, Thermotogaceae, Treponema, Xanthomonas, Vibrio, Vogesella, and Yersinia.

In preferred embodiments, microbes for use in the claimed methods are bacteria, either Gram negative or Gram positive bacteria. More specifically, the present invention contemplates any bacteria that are shown to be infectious or potentially infectious.

Examples of microbes suitable for testing or treating include, but are not limited to, Acinetobacter calcoaceticus, Acinetobacter haemolyticus, Aeromonas hydrophilia, Agrobacterium tumefaciens, Bacillus anthracis, Bacillus halodurans, Bacillus subtilis, Bacteroides distasonis, Bacteroides eggerthii, Bacteroides fragilis, Bacteroides ovalus, Bacteroides 3452A homology group, Bacteroides splanchnicus, Bacteroides thetaiotaomicron, Bacteroides uniformis, Bacteroides vulgatus, Bordetella bronchiseptica, Bordetella parapertussis, Bordetella pertussis, Borrelia burgdorferi, Branhamella catarrhalis, Brucella melitensis, Burkholderia cepacia, Burkholderia pseudomallei, Campylobacter coli, Campylobacterfetus, Campylobacter jejuni, Caulobacter crescentus, Citrobacter freundii, Clostridium difficile, Clostridium perftingens, Corynebacterium diphtheriae, Corynebacterium glutamicum, Corynebacterium ulcerans, Edwardsiella tarda, Enterobacter aerogenes, Erwinia chrysanthemi, Enterobacter cloacae, Enterococcus faecalis, Enterococcus faecium, Escherichia coli, Francisella tularensis, Gardnerella vaginalis, Haemophilus ducreyi, Haemophilus haemolyticus, Haemophilus influenzae, Haemophilus parahaemolyticus, Haemophilus parainfluenzae, Helicobacter pylori, Klebsiella oxytoca, Klebsiella pneumoniae, Kluyvera cryocrescens, Legionella pneumophila, Listeria innocua, Listeria monocytogenes, Listeria welshimeri, Methanosarcina acetivorans, Methanosarcina mazei, Morganella morganii, Mycobacterium avium, Mycobacterium intracellulare, Mycobacterium leprae, Mycobacterium tuberculosis, Mesorhizobium loti, Neisseria gonorrhoeae, Neisseria meningitidis, Pasteurella haemolytica, Pasteurella multocida, Providencia alcalifaciens, Providencia rettgeri, Providencia stuartii, Proteus mirabilis, Proteus vulgaris, Pseudomonas acidovorans, Pseudomonas aeruginosa, Pseudomonas alcaligenes, Pseudomonasfluorescens, Pseudomonas putida, Ralstonia solanacearum, Salmonella enterica subsp. enteridtidis, Salmonella enterica subsp. paratyphi, Salmonella enterica, subsp. typhimurium, Salmonella enterica, subsp. typhi, Serratia marcescens, Shigella dysenteriae, Shigella flexneri, Shigella sonnei, Sinorhizobium meliloti, Staphylococcus aureus, Streptococcus criceti, Staphylococcus epidemmidis, Staphylococcus haemolyticus, Staphylococcus hominis, Staphylococcus hyicus, Staphylococcus intermedius, Stenotrophomonas maltophilia, Staphylococcus saccharolyticus, Staphylococcus saprophyticus, Staphylococcus sciuri, Streptomyces avermitilis, Streptomyces coelicolor, Streptococcus agalactiae, Streptococcus pneumoniae, Streptococcus pyogenes Sulfobalblobus soffiataricus, Thermotoga maritima, Vibrio cholerae, Vibrio parahaemolyticus, Vogesella indigofera, Xanthomonas axonopodis, Xanthomonas campestris, Yersinia enterocolitica, Yersinia intermedia, Yersinia pestis, and Yersinia pseudotuberculosis.

In other embodiments, the microbes to be tested or treated are fungi. In a preferred embodiment the fungus is from the genus Mucor, Aspergillus, or Candida, e.g., Mucor racmeosus, Aspergillus fumigatus, and Candida albicans.

In yet other embodiments, the microbes to be tested or treated are protozoa. In a preferred embodiment the microbe is a malaria, trypansome, giardia, or cryptosporidium parasite.

In yet another embodiment, the microbes to be tested or treated are viruses. There are a number of viruses that are recognized and affected by the innate immune system, particularly by macrophage activity. Viruses that are contemplated include, but are not limited to, retroviruses proviruses, lentivriurses such as immunodeficiency viruses, togaviruses. Togaviruses, as used herein includes the Togaviridae family, including the Alphavirus and Rubivirus genera, as well as the flavivirus family (Flaviviridae) and the Flavivirus and Pestivirus genera. A review of virus taxonomy and the biology of these viruses may be found at, e.g., B. N. Fields, et al., editors, Fundamental Virology, 3rd edition, 1996, Lippencott-Raven Publishers, chapter I (pages 15-58) and chapter 17 (pages 523-540), which is incorporated herein by reference.

In another embodiment, the microbe of concern is a potential bioterrorism agent. For example, in one embodiment, one or more of the microbes selected from the group consisting of: Bacillus anthracis (anthrax); Clostridium botulinum; Yersinia pestis (plague); Francisella tularensis (tularemia); Burkholderia pseudomallei; Coxiella burnetti (Q fever); Brucella species (brucellosis); Burkholderia mallei (glanders); Epsilon toxin of Clostridium perfringens; Staphylococcus enterotoxin B; Typhus fever (Rickettsia prowazekii); Diarrheagenic E. coli; Pathogenic Vibrios (e.g., V. parahaemolyticus, V. vuliificus, V. mimicus, V. hollisae, V. fluvialis, V alginolyticus, V. metschnikovii, and V. damsela; Shigella spp.; Salmonella spp.; Listeria monocytogenes; Campylobacter jejuni; Yersinia enterocolitica; Multi-drug resistant Mycobacterium tuberculosis; Other Rickettsias (e.g., R. rickettsii, R. conorii, R. tsutsugamushi, R. typhi, and R. akari), or combinations/derivatives thereof; is targeted in the subject assays or is treated using a compound or method of the invention.

Macrophages are one population of effector cells involved in immune responses. Their role in natural immunity includes mediation of phagocytosis, as well as release of cytokines and cytotoxic mediators. They also facilitate the development of acquired immunity through antigen presentation and release of immunomodulatory cytokines. Although macrophages are immune effectors, they are also susceptible to infection by agents such as bacteria, protozoa, parasites, and viruses (The Macrophage, C. E. Lewis & J. O'D. McGee. eds., IRL Press at Oxford University Press, New York, N.Y., 1992). Viruses capable of infecting macrophages include several RNA viruses such as measles virus (Mv) (e.g., Joseph et al., J. Virol. 16, 1638-1649, 1975), respiratory syncytial virus (RSV) (Midulla et al., Am. Rev. Respir. Dis. 140, 771-777, 1989), and human immunodeficiency virus type 1 (HIV-1) (Meltzer and Gendelman, in Macrophage Biology and Activation, S. W. Russell and S. Gordon, eds., Springer-Verlag, New York, N.Y., pp. 239-263@ 1992: Potts et al., Virology 175, 465-476, 1990).

In addition to pathogen infection and virulence, other diseases and conditions are contemplated by the present invention. One aspect of the present invention is the increasing of the killing capacity of the cells of the innate immune system of a subject. Those of skill in the art will readily recognize other diseases and conditions which can be treated employing the methods and compositions of the present invention. Included in the present invention includes various diseases, non-bacterial in origin, associated with a high incidence of complications due to infection. Examples of such diseases include end-stage renal disease (Goldblum and Reed, Ann. Intern. Med. 93:597 (1980); Lahnborg et al., Transplantation 28:111 (1979); Drivas et al., Invest. Urol. 17:241 (1979); Keane and Raij, In: Drukkar et al., Eds. Replacement of Renal Function by Dialysis, 2nd ed., pp. 646-58 (1983)), acquired immunodeficiency syndrome (AIDS) (Bender et al., J. Infect. Disease 152:409 (1985), Smith et al., J. Clin. Invest. 74:2121 (1984)), liver disease (Rimola, In: McIntyre et al. Eds Oxford Textbook of Clinical Hepatology, pp. 1272-84 (1991)) and diseases of the lung, including cystic fibrosis (Gomez and Schreiber, unpublished observations) and acute respiratory distress syndrome (ARDS) (Rossman et al., Clin. Res. 41:251A (1993)). In addition, those of skill in the art recognize the present invention can be employed to treat cancers. Unlike the prior art which has employed HIF-1 antagonists to limit cancerous growth, the present invention is directed to the reduction or clearance of tumors or cancerous cells via the innate immune system by employing HIF-1 agonists.

### Assays

In one embodiment, the invention provides for methods of identifying test compound(s) which bind or disrupt HIF-1 or HIF-1 interacting proteins, or modulate HIF-1 activity. The term modulate includes both up-regulating (i.e., turning on or increasing) and down regulating (i.e., turning off or decreasing) expression or activity. Thus, HIF-1 may be employed in a screening process for compounds which directly or indirectly bind, interact, disrupt, activate (agonists) or inhibit activation (antagonists) of this transcription factor, its interacting proteins or responsive element, and/or the genes that it regulates (e.g., HIF-1 subunit(s) or complex, a HIF interacting compound such as vHL, IPAS, ARD-1, or HIF hydroxylase (e.g., PHD1, PHD2, PHD3, FIH1 (see, e.g., Epstein et al., (2001) Cell 107, 43-54 and Mahon et al., (2001) Genes Dev. 15:2675-2686), a hypoxia response element (HRE), iNOS, cathelicidin such as CRAMP, and the like.). The ability of the test compound to bind HIF-1/HIF-1 interacting proteins/responsive element or modulate an activity of HIF-1 can be determined in a variety of ways, as outlined in more detail below. Such assays may be for many uses including the development of drug candidates, for diagnostic purposes or for the gathering of information for therapeutics. The unique mouse reagents with targeted gene deletions in the genes encoding HIF-1 and its counter-regulatory agent vHL allow us to determine specifically that any observed effects on gene regulation are HIF-specific.

Accordingly, in one series of embodiments, the present invention provides methods of screening or identifying proteins, small molecules or other compounds which are capable of inducing or inhibiting the expression of the HIF-1 or HIF-1 interacting proteins, or increasing (accumulating), stabilizing or decreasing (degrading) the level of HIF-1 genes and proteins. The assays may be performed in vitro using transformed or non-transformed cells, immortalized cell lines, or in vivo using the transgenic animal models or human subjects described herein. In particular, the assays may detect the presence of increased or decreased expression/accumulation/degradation of HIF-1 or other HIF-1-interacting genes or proteins on the basis of increased or decreased mRNA expression, increased (including non-degraded) or decreased levels of HIF-1 interacting protein products, or increased or decreased levels of expression of a selectable marker/reporter gene (e.g., beta-galactosidase, green fluorescent protein, alkaline phosphatase or luciferase) (which are/were operably joined to a HIF-1 5′ regulatory region in a recombinant construct). For example, the expression of HIF-1 can be upregulated independently of hypoxia through alteration of pathways such as the PI3K/AKT and Ras/MAPK signal transduction pathways. Cells known to express a particular HIF-1, or transformed to express a particular HIF-1, are incubated and one or more test compounds are added to the medium. After allowing a sufficient period of time (e.g., 0-72 hours) for the compound to induce or inhibit the expression of the HIF-1, any change in levels of expression from an established baseline may be detected using any of the techniques known in the art.

In general, a cell may be contacted with a candidate compound and, after an appropriate period (e.g., 0-72 hours for most biochemical measures of cultured cells), the marker of activity may be assayed and compared to a baseline measurement. The baseline measurement may be made prior to contacting the cell with the candidate compound or may be an external baseline established by other experiments or known in the art. The cell may be a transformed cell or an explant from an animal or individual. To augment the effect, transgenic cells or animals may be employed which have increased production. Preferred cells include human or murine macrophage cells transfected with HRE-reporter constructs. The cells may be contacted with the candidate compounds in a culture in vitro or may be administered in vivo to a live animal or human subject. For live animals or human subjects, the test compound may be administered orally or by any parenteral route suitable to the compound. For clinical trials of human subjects, measurements may be conducted periodically (e.g., daily, weekly or monthly) for several days, weeks, months or years.

A number of proteins are known to induce HIF-1α protein translation irrespective of hypoxia, including certain growth factors (see, e.g., Lee et al., Exp Mol Med 36(1):1-12 (2004), including the EBV latent membrane protein 1 (LMP1) (Wakisaka et al., Mol Cell Biol 24(12):5223-34 (2004)), and the like. Of particular interest are methods of screening proteins, small molecules or other compounds which are capable of increasing the accumulation or stability of HIF-1, or decreasing degradation of HIF-1 by inhibiting the interaction of HIF-1, particularly HIF-1α, with HIF hydroxylases and/or vHL, thus inhibiting HIF-1αdegradation and/or promoting HIF-1α accumulation/stability. Those of skill will readily recognize methods for identifying such compounds, including readily employing the techniques described below in greater detail.

In another series of embodiments, the present invention provides methods for identifying proteins and other compounds that bind to, or otherwise directly interact with HIF-1, HIF-1 interacting protein and/or HIF responsive elements. The proteins and compounds will include endogenous cellular components which interact with HIF-1 in vivo and which, therefore, provide new targets for pharmaceutical and therapeutic interventions, as well as recombinant, synthetic and otherwise exogenous compounds which may have HIF-1 binding capacity and, therefore, may be candidates for pharmaceutical agents. Thus, in one series of embodiments, cell lysates or tissue homogenates (e.g., human homogenates, lymphocyte lysates) may be screened for proteins or other compounds which bind to HIF-1, HIF-1 interacting protein and/or HIF responsive elements. Alternatively, any of a variety of exogenous compounds, both naturally occurring and/or synthetic (e.g., libraries of small molecules or peptides), may be screened for HIF-1, HIF-1 interacting protein and/or HIF responsive element binding capacity. In each of these embodiments, an assay is conducted to detect binding between a “HIF-1 component” (HIF-1, HIF-1 interacting protein and/or HIF responsive elements) and some other moiety. The HIF-1 component” in these assays may be any polypeptide comprising or derived from HIF-1, including functional subunits, domains or antigenic determinants of HIF-1, or HIF-1 interacting protein such as vHL, HIF hydroxylases or HREs.

Methods for screening cellular lysates, tissue homogenates, or small molecule libraries for candidate complex-specific or component-specific binding compounds are well known in the art and, in light of the present disclosure, can be employed to identify compounds which bind specifically to the complex or the individual and separate components or which modulate complex activity as defined by non-specific measures (e.g., changes in intracellular signaling, transcription, and the like) or by specific measures (e.g., changes in downstream peptide production, altered chromatin structure, peptide production or changes in the expression of other downstream genes which can be monitored by differential display, gel electrophoresis, differential hybridization, or serial analysis of gene expression (SAGE) methods). Specific embodiments contemplated by the present invention include variations on the following techniques: (1) direct extraction by affinity chromatography; (2) immunocytochemical experiments; (3) the Biomolecular Interaction Assay (BIAcore); (4) the yeast one-, two- or three hybrid systems, (5) GST-tag pull down assays, (6) co-immunoprecipitation and the like which may, measure, for example, a change in fluorescence, molecular weight, or concentration of either the binding agent, HIF-1, HIF-1 interacting component, or HRE either in a soluble phase or in a substrate-bound phase. As will be appreciated by one of ordinary skill in the art, there are numerous other methods of screening individual proteins or other compounds, as well as large libraries of proteins or other compounds (e.g., phage display libraries and cloning systems from Stratagene, La Jolla, Calif.) to identify molecules which specifically bind to the complex or the components. For example, some methods generally combine the steps of mixing either the complex or component(s) (fusion or fragment) with test compounds, allowing for binding (if any), and assaying for bound complexes.

A binding response could be measured by testing for the adherence of a test compound to HIF-1 or a HIF interacting protein borne on a solid surface, borne on a cell surface, free in solution or located intracellularly. The test compound may aid polypeptide detection by being labeled, either directly or indirectly. Alternatively, the polypeptide itself may be labeled, for example, with a radioisotope, by chemical modification or as a fusion with a peptide or polypeptide sequence that will facilitate polypeptide detection. Alternatively, a binding response may be measured, for example, by performing a competition assay with a labeled competitor or vice versa. One example of such a technique is a competitive drug-screening assay, where neutralising antibodies that are capable of specifically binding to the polypeptide compete with a test compound for binding. In this manner, the antibodies may be used to detect the presence of any test compound that possesses specific binding affinity for the polypeptide. Alternative binding assay methods are well known in the art and include, but are not limited to, cross-linking assays and filter binding assays. The efficacy of binding may be measured using biophysical techniques including surface plasmon resonance and spectroscopy.

These assays may also be used to screen many different types of compounds for their disruptive effect on the interactions of HIF-1. For example, the compounds may belong to a library of synthetic molecules, or be specifically designed to disrupt the interaction of HIF-1 and either HIF-1 interacting protein or HREs. The compounds may also be peptides corresponding to the interacting domain of these factors. This type of assay can be used to identify compounds that disrupt a specific interaction between a given HIF-1 variant and a given interacting protein. In addition, compounds that disrupt all HIF-1 interactions may be identified. For example, a compound that specifically disrupts the folding of HIF-1 proteins would be expected to disrupt all interactions between HIF-1 and other proteins. Alternatively, this type of disruption assay can be used to identify compounds that disrupt only a range of different HIF-1 interactions (e.g., HIF hydroxylases), or only a single HIF-1 interaction (e.g., vHL or HRE).

In another series of embodiments, the present invention provides for methods of identifying proteins, small molecules and other compounds capable of modulating the activity of HIF-1s. As used with respect to this series of embodiments, the term “activity” broadly includes gene and protein expression, HIF-1 protein post-translation processing, trafficking and localization, and any functional activity (e.g., enzymatic, receptor-effector, binding, transcriptional), as well as downstream affects of any of these. In a particular aspect of the present invention, there are provided methods for identifying compounds, such as ligands, capable of directly or indirectly modulating the activity of HIF-1α. Using normal cells or animals or, transformed cells and animal models, the present invention provides methods of identifying such compounds on the basis of their ability to affect the expression of the HIF-1s, the intracellular localization of the HIF-1s, changes in transcription activity, or other metabolic measures, the occurrence or rate of degradation of HIF-1, the levels or pattern of HIF-1 or the genes that it regulates, or other biochemical, histological, or physiological markers which distinguish compounds capable of modulating activity or level of HIF-1. Using animal models (e.g., HIF-1 knock-outs, vHL knock-outs and/or HIF hydroxylase knock-outs), methods of identifying such compounds are also provided on the basis of the ability of the compounds to affect behavioral, physiological or histological phenotypes.

HIF-1 can be used to screen libraries of compounds in any of a variety of drug screening techniques. Such compounds may activate (agonise) or inhibit (antagonize) the level of expression of the gene or the activity of HIF-1 or HIF-1 interacting proteins/HREs and form a further aspect of the present invention. Methods for doing this include the screening of a library of compounds (see Coligan et al., Current Protocols in Immunology 1(2); Chapter 5 (1991), isolating the ligands from cells, isolating the ligands from a cell-free preparation or natural product mixtures. Ligands of the invention may activate (agonise) or inhibit (antagonize) HIF-1 activity. Alternatively, compounds may affect the levels of HIF-1 present in the cell, including affecting gene expression, mRNA and protein stability and the degree of post-translational modification of HIF-1.

Thus, the present invention provides methods for screening or assaying for proteins, small molecules or other compounds which modulate HIF-1 activity by contacting a cell in vivo or in vitro with a candidate compound and assaying for a change in a marker associated with HIF-1 activity. The marker associated with HIF-1 activity may be any measurable biochemical, physiological, histological and/or behavioral characteristic associated with HIF-1 activity, whether natural or synthetic. Such measures include specific measures of expression of downstream genes (e.g., iNOS, cathelicidin or TNF-α (mRNA or protein levels)), or selective markers/reporter genes, or non-specific measures including changes in cell physiology such as phagocytic uptake of bacterium or other pathogen, generation of phagolysomes, production of reactive oxygen species, release of antimicrobial peptides (e.g., cathelicidin, defensins) and granule proteases (e.g., elastase, cathepsin), etc., which can be monitored on devices such as the cytosensor microphysiometer (Molecular Devices Inc., United States). These can also be assayed by such techniques as high-density microarrays (e.g. Affymetrix “Gene Chip”), differential display, differential hybridization, and SAGE (sequential analysis of gene expression), as well as by proteomic analysis two dimensional gel electrophoresis of cellular lysates, or other techniques known in the art such as exploiting spectral or other physical markers. In each case, the differentially-expressed genes can be ascertained by inspection of identical studies before and after application of the candidate compound. Furthermore, as noted elsewhere, the particular genes whose expression is modulated by the administration of the candidate compound can be ascertained by cloning, nucleotide sequencing, amino acid sequencing, or mass spectrometry (reviewed in Nowak, 1995). The unique reagents with targeted gene deletions in the genes encoding HIF-1 and its counter-regulatory agent vHL allow us to determine specifically that any observed effects on protein expression or immune cell function are HIF-specific.

In one embodiment, the assays described herein can employ indicators, such as selective markers and reporter genes. The term selective marker includes polypeptides that serve as indicators, e.g., provide a selectable or screenable trait when expressed by a cell. The term “selective marker” includes both selectable markers and counterselectable markers. As used herein the term “selectable marker” includes markers that result in a growth advantage when a compound or molecule that fulfills the test parameter of the assay is present. The term “counterselectable marker” includes markers that result in a growth disadvantage unless a compound or molecule is present which disrupts a condition giving rise to expression of the counterselectable marker. Exemplary selective markers include cytotoxic gene products, gene products that confer antibiotic resistance, gene products that are essential for growth, gene products that confer a selective growth disadvantage when expressed in the presence of a particular metabolic substrate (e.g., the expression of the URA3 gene confers a growth disadvantage in the presence of 5fluoroorotic acid).

In particularly preferred embodiments, a recombinant assay is employed in which a reporter gene such a β-galactosidase, green fluorescent protein, alkaline phosphatase, or luciferase is operably joined to a hypoxic response element (HRE). The hypoxic regulatory regions disclosed herein, or other HIF-1 regulatory regions, may be easily isolated and cloned by one of ordinary skill in the art. Preferably, the regulatory region is cloned upstream of the intact reporter gene at the appropriate distance so that transcription and translation of the reporter gene may proceed under the control of the hypoxic regulatory elements The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high through-put assay for the identification of inducers and repressors of the HIF-1 gene. Alternatively, compounds that potentiate the activity of HIF-1, particularly HIF-1α, by antagonizing certain HIF interacting proteins such as vHL, hydroxylases, ARD-1, and the like, are of interest as lead compounds. Compounds identified by this method will have potential utility in modifying the activity of HIF-1. These compounds may be further tested in the animal models disclosed and enabled herein to identify those compounds having the most potent in vivo effects. In addition, as described herein with respect to small molecules having HIF-1-binding activity, these molecules may serve as “lead compounds” for the further development of pharmaceuticals by, for example, subjecting the compounds to sequential modifications, molecular modeling, structure based drug design, drug screening using high-throughput and ultra high-throughput screening assays, combinatorial chemistry, and other routine procedures employed in drug development and design.

High throughput assays for the presence, absence, or quantification of particular nucleic acids or protein products are well known to those of skill in the art. Similarly, binding assays and reporter gene assays are similarly well known, as are high throughput screening methods for proteins, high throughput screening methods for nucleic acid binding (i.e., in arrays), and methods of screening for ligand/antibody binding. In addition, high throughput screening systems are commercially available. Any of the assays for compounds modulating HIF-1 gene expression and/or HIF-1 protein activity described herein or previously described are amenable to high throughput screening. Preferred assays thus detect enhancement or inhibition of HIF-1 gene transcription, inhibition or enhancement of HIF-1 polypeptide expression, inhibition or enhancement of DNA binding by HIF-1 polypeptide, inhibition or enhancement of protein interaction with HIF-1, inhibition or enhancement of HIF-1 degradation, inhibition or enhancement of HIF-1 cytoplasmic translocation, or inhibition or enhancement of expression of native genes (or reporter genes) under control of the HIF-1 polypeptide.

The proteins or other compounds identified by these methods may be purified and characterized by any of the standard methods known in the art. Proteins may, for example, be purified and separated using electrophoretic (e.g., SDS-PAGE, 2D PAGE) or chromatographic (e.g., HPLC) techniques and may then be microsequenced. For proteins with a blocked N-terminus, cleavage (e.g., by CNBr and/or trypsin) of the particular binding protein is used to release peptide fragments. Further purification/characterization by HPLC and microsequencing and/or mass spectrometry by conventional methods provides internal sequence data on such blocked proteins. For non-protein compounds, standard organic chemical analysis techniques (e.g., IR, NMR and mass spectrometry; functional group analysis; X-ray crystallography) may be employed to determine their structure and identity.

Once identified by the methods described above, the candidate compounds may then be produced in quantities sufficient for pharmaceutical administration or testing (e.g., μg or mg or greater quantities), and formulated in a pharmaceutically acceptable carrier (see, e.g., Remington's Pharmaceutical Sciences, Gennaro, A., ed., Mack Pub., 1990). These candidate compounds may then be administered to transformed cells, to transgenic animal models, to cell lines derived from the animal models or from human patients, or to subjects. The animal models described and enabled herein are of particular utility in further testing candidate compounds which bind to HIF-1 for their therapeutic efficacy.

Compounds for testing in the instant methods can be derived from a variety of different sources and can be known (although not previously known to modulate the activity and/or expression of transcription factors) or can be novel. In one embodiment, libraries of compounds are tested in the instant methods to identify HIF-1 modulating compounds. In another embodiment, known compounds are tested in the instant methods to identify HIF-1 modulating compounds (as used herein, the term “HIF-1 modulating compound” refers to compounds which modulate the level and/or activity of HIF-1 or HIF-1 interacting proteins). In an embodiment, compounds among the list of compounds generally regarded as safe (GRAS) by the Environmental Protection Agency are tested in the instant methods.

In one embodiment, a plurality of test compounds is tested using the disclosed methods. In another embodiment, the compounds tested in the subject screening assays were not previously known to modulate transcription factor activity. Exemplary compounds that can be screened for activity include, but are not limited to, peptides, nucleic acids, carbohydrates, small organic molecules, and natural product extract libraries. In one embodiment, the test compound is a peptide or peptidonimetic. In another, preferred embodiment, the compounds are small, organic non-peptidic compounds.

The term “test compound” includes any reagent or test agent which is employed in the assays of the invention and assayed for its ability to influence the activity of HIF-1, e.g., a HIF-1α, HIF-1β, HIF-1 complex, or e.g., by binding to the polypeptide or to a molecule with which it interacts, i.e., v. vHL. vHL inhibitors are well known in the art and can be readily employed as lead compounds or actual compounds of the present invention. More than one compound, e.g., a plurality of compounds, can be tested at the same time for their ability to modulate the activity of HIF-1, e.g., a HIF-1α, HIF-1β, HIF-1 complex, in a screening assay. In one embodiment, high throughput screening can be used to assay for the activity of a compound. In one embodiment, test compounds are selected from HIF-1 modulating compound, vHL modulating compounds, or HIF-1 hydroxylase modulating compounds. Test compounds that can be tested in the subject assays include antibiotic and non-antibiotic compounds. As used herein, the term “antibiotic” includes antimicrobial agents isolated from natural sources or chemically synthesized, preferably to agents for use in human therapy.

In one embodiment, test compounds include candidate detergent or disinfectant compounds. Exemplary test compounds which can be screened for activity include, but are not limited to, peptides, non-peptidic compounds, nucleic acids, carbohydrates, small organic molecules (e.g., polyketides), and natural product extract libraries. The term “non-peptidic test compound” includes compounds that are comprised, at least in part, of molecular structures different from naturally occurring L-amino acid residues linked by natural peptide bonds. However, “non-peptidic test compounds” also include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic test compounds” also are intended to include natural products.

In one embodiment, small molecules can be used as test compounds. Small molecules are particularly preferred in this context because they are more readily absorbed after oral administration, have fewer potential antigenic determinants, and/or are more likely to cross the blood barrier than larger molecules such as nucleic acids or proteins. The term “small molecule” is a term of the art and includes molecules that are less than about 1000 molecular weight or less than about 500 Daltons (Da) molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), natural product extract libraries and products by combinatorial chemistry techniques. In another embodiment, the compounds are small, organic non-peptidic compounds. In a further embodiment, a small molecule is not biosynthetic.

A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al. 1992. J. Am. Chem. Soc. 114:10987; DeWitt et al. 1993. Proc. Natl. Acad. Sci. USA 90:6909) peptoids (Zuckermann. 1994. J. Med. Chem. 37:2678) oligocarbamates (Cho et al. 1993. Science. 261:1303), and hydantoins (DeWitt et al. supra). Rebek et al. have described an approach for the synthesis of molecular libraries of small organic molecules with a diversity of self-assembling systems (Carell et al. 1994. Angew. Chem. Int. Ed. Engl. 33:2059; Carell et al. Angew. Chem. Int. Ed. Engl. 1994. 33:2061). See, generally, Nicolaou, K. C. et al. (Eds), Handbook of Combinatorial Chemistry: Drugs, Catalysts, Materials (Wiley-VCH, Weinheim, 2002), incorporated herein by reference.

Ligands to HIF-1 form a further aspect of the invention. Preferred “agonist” ligands include those that bind to the polypeptide HIF-1 or HIF-1 interacting proteins and strongly induce activity of the polypeptide and/or increases or maintain substantially the level of the polypeptide in the cell, e.g., by binding to and activating HIF-1, by binding to a nucleic acid target with which the transcription factor interacts, by facilitating or disrupting a signal transduction pathway responsible for activation of a particular regulon, and/or by facilitating or disrupting a critical protein-protein interaction (e.g., facilitating HIF-1 complex or disrupting vHL or HIFPH binding, respectively). “Antagonist” ligands include those that bind to the polypeptide HIF-1 or HIF-1 interacting proteins and strongly inhibit any activity of the polypeptide by binding to and inactivating HIF-1 or the HIF-1 interacting protein, e.g., by binding to a nucleic acid target with which the transcription factor interacts (e.g., HRE), by facilitating or disrupting a signal transduction pathway responsible for activation of a particular regulon, by facilitating or disrupting a critical protein-protein interaction (e.g., facilitating HIF-1 complex or disrupting vHL or HIF-1 hydroxylase binding, respectively), and/or increasing the degradation of proteins (e.g., facilitating vHL or other hydroxylase mediated HIF-1 degradation). As defined above, the term “ligand”, “agonist” or “antagonist” is meant to include any polypeptide, peptide, synthetic molecule or organic molecule that functions as an modulator, activator, or inhibitor of HIF-1 activity, respectively.

Ligands according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, and structural or functional mimetics of the aforementioned. Agonist or antagonist compounds may be isolated from, for example, cells, cell-free preparations, chemical libraries or natural product mixtures.

Once identified by the methods described above, the candidate compounds may also serve as “lead compounds” in the design and development of new pharmaceuticals. Accordingly, the search for lead compounds includes an analysis of compound banks, for example, available commercial, custom, or natural products chemical libraries, or a combinatorial chemical library, e.g., a collection of diverse chemical compounds generated by chemical synthesis or biological synthesis.

With combinatorial chemistry millions of organic compounds can be produced simultaneously, quickly, and in most cases by automated procedures. These compound libraries are a cost-effective resource to search for biologically active lead structures. Furthermore simultaneous parallel synthesis of single peptides and peptide libraries solve the problem of the worldwide increasing demand for peptides. The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, synthetic library methods using affinity chromatography selection, and other various synthetic approaches and technologies, mass spectrometry, and screening assays (see generally, Jung, Günther. Combinatorial Peptide and Nonpeptide Libraries: A Handbook (John Wiley & Sons; 1997); Abraham, D. J., Burger's Medicinal Chemistry and Drug Discovery, Drug Discovery and Drug Development (Wiley-Interscience; 6th edition 2003).

For example, as is well known in the art, sequential modification of small molecules (e.g., amino acid residue replacement with peptides; functional group replacement with peptide or non-peptide compounds) is a standard approach in the pharmaceutical industry for the development of new pharmaceuticals. Such development generally proceeds from a lead compound which is shown to have at least some of the activity (e.g., HIF-1 binding or blocking ability) of the desired pharmaceutical. In particular, when one or more compounds having at least some activity of interest (e.g., modulation of HIF-1 activity) are identified, structural comparison of the molecules can greatly inform the skilled practitioner by suggesting portions of the lead compounds which should be conserved and portions which may be varied in the design of new candidate compounds. Thus, the present invention also provides a means of identifying lead compounds that may be sequentially modified to produce new candidate compounds for use in the treatment of pathogen infection or virulence. These new compounds then may be tested both for HIF-1-binding or blocking (e.g., in the binding assays described above) and for therapeutic efficacy (e.g., in animal models). This procedure may be iterated until compounds having the desired therapeutic activity and/or efficacy are identified.

Of particular interest are compounds currently identified as HIF-1 interacting or HIF-1 modulating compounds (directly or indirectly, e.g., via HIF-1 interacting proteins), or compounds identified utilizing methods described or known to those of skill in the art. For example, for hypoxia-regulated polypeptides implicated herein which are active as prolyl 4-hydroxylases, examples of suitable ligand compounds for screening in the above assays or as lead compounds include substrate-based inhibitors, such as 3-exomethyleneproline peptide like compounds (Tandon M et al., Bioorg. Med. Chem. Lett. 8:1139-44 (1998)), derivatives of proline, derivatives of 4(S)hydroxy proline, and derivatives of 4-keto proline. Furthermore, in view of the fact that the activity of proline-4-hydroxylase is iron, 2-oxoglutarate and ascorbic acid dependent (Kivirikko K I, Pihlajaniemi T. Adv Enzymol Relat Areas Mol Biol. 72:325-98 (1998)) and the activity of HIF targeting prolyl hydroxylases such as those recited above is also dependent on these co-factors (Bruick R K, McKnight S L. Science. 294(5545):1337-40 (2001)), examples of suitable compounds include cofactor-based inhibitors such as 2-oxoglutarate analogues, ascorbic acid analogues and iron chelators such as desferrioxamine (DFO) and the hypoxia mimetic cobalt chloride (CoCl₂), or other factors that may mimic hypoxia. Also, of interest as compounds suitable for the present invention are hydroxylase inhibitors, including as lead compounds, includes deferiprone, 2,2′-dipyridyl, ciclopirox, dimethyloxallyl glycine (DMOG), L-Mimosine (Mim) and 3-Hydroxy-1,2-dimethyl-4(1H)-Pyridone (OH-pyridone). DMOG is a cell permeable, competitive inhibitor of HIF-PH. It acts to stabilize HIF-1α expression at normal oxygen tensions in cultured cells, at concentrations between 0.1 and 1 mM. Other HIF hydroxylase inhibitors are described herein, including but not limited to, oxoglutarates, heterocyclic carboxamides, phenanthrolines, hydroxamates, and heterocyclic carbonyl glycines (including, but not limited to, pyridine carboxamides, quinoline carboxamides, isoquinoline carboxamides, cinnoline carboxamides, beta-carboline carboxamides, including substituted quinoline-2-carboxamides and esters thereof; substituted isoquinoline-3-carboxamides and N-substituted arylsulfonylamino hydroxamic acids (see, e.g., PCT Application No. WO 05/007192, WO 03/049686 and WO 03/053997), and the like. Also of interest are compounds described or identified using the methods described in the art, including U.S. Pat. No. 6,787,326, and 6,767,705, and 6,436,654; U.S. Provisional Application Nos. 2004/0161794, 2004/0152655, 2004/0146964, 2004/0096848, 2004/0087556, 2003/0229108 and 2002/0048794; and PCT Application Nos. WO 04/066949, WO 04/047852, WO 04/043359, WO 04/000328, WO 03/100438, WO 03/085110, WO 03/080566, WO 03/074560, WO 03/049686, WO 03/018014, WO 02/12326, and WO 02/074981 (each herein incorporated by reference). Other compounds of interest will be known or discovered in the art by the teachings described herein, including:

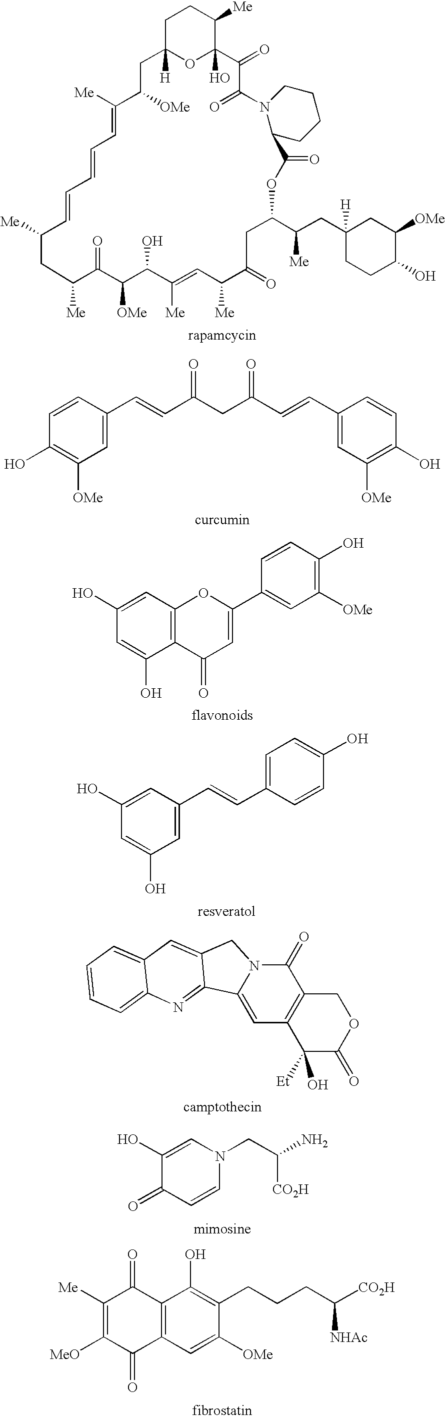

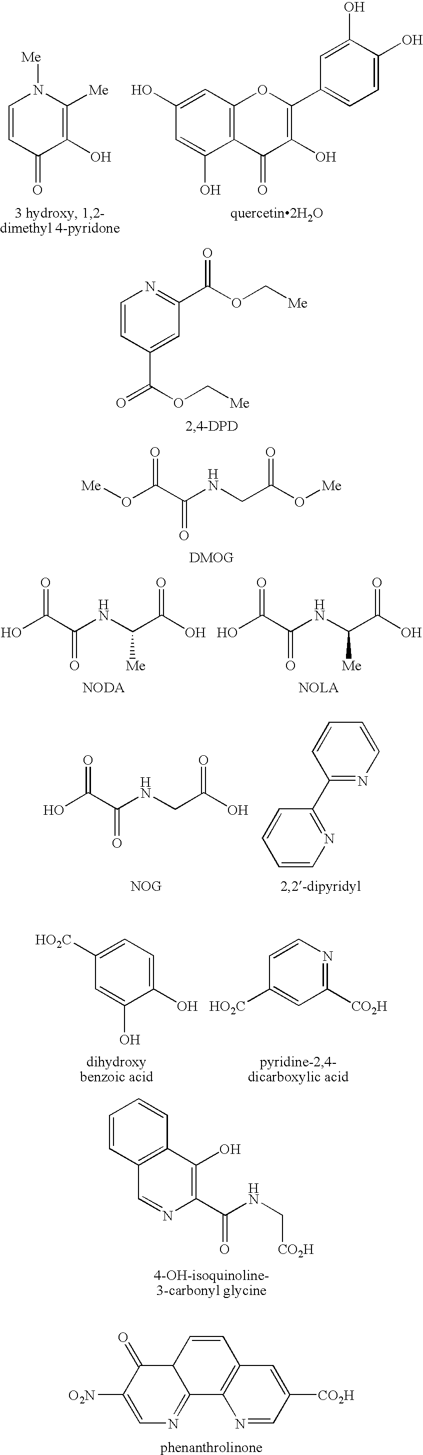

More specifically, compounds which interact or modulate HIF-1 or HIF-1-interacting compounds could be readily applied or modified and evaluated to determine their applicability in the present invention. A general report of such compounds and the pathways associated with HIF-1 levels and HIF-1 activity are disclosed in Semenza, Nature Rev. Cancer 2003, 721, Ratcliffe et al; Nature Medicine, 2003, 677, and Wouters et al., Drug Resistance Updates 2004, 25 (each herein incorporated by reference). Other suitable compounds which can be used to derive desirable compounds include rapamycin (see, e.g., Abraham, R. T. Current Topics in Microbiology and Immunology (2004), 279, 299-319; Arsham et al., Journal of Biological Chemistry (2003), 278(32), 29655-29660), curcumin (see, e.g., Sukhatme, V P. PCT Int. Appl. (2003), WO 03/094904), fibrostatin (Ishimaru et al., Journal of Antibiotics (1988), 41(11), 1668-74), mimosine (see, e.g., Warnecke et al., FASEB Journal (2003), 17(9), 1186-1188; Park, et al., WO03/018014; Clement et al., International Journal of Cancer (2002), 100(4), 491-498), 3 hydroxy, 1,2 dimethyl 4-pyridone (see, e.g., Weidmann et al., WO 97/41103; Weidmann et al., EP/650961; Iyer et al., Experimental lung research (1998 January-February), 24(1), 119-32), camptothecin (see, e.g., Rapisarda et al., Cancer Research (2002), 62(15), 4316-4324), resveratrol (see, e.g., Cao et al., Clinical cancer research: an official journal of the American Association for Cancer Research (2004 Aug. 1), 10(15), 5253-63), Flavonoids (see, e.g., Hasebe et al., Biological & Pharmaceutical Bulletin (2003), 26(10), 1379-1383; Fan et al., Eur J Pharm (2003), 481(1), 33-40); Majamaa et al. (1984) Eur J Biochem 138:239-245; and Majamaa et al. (1985) Biochem J229: 127-133; Kivirikko and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19):812-815; and Franklin et al. (2001) Biochem J 353:333-338; and the like (each reference herein incorporated). A number of HIF-1 modulators have been identified by those skilled in the art for a number of disorders including anemia and neoplasias. Of particular interest are compounds and the methods employed to identify such compounds, particularly compounds that stabilize HIF-1α, such as those identified in PCT application No. WO 04/108121. For example, compounds including [(3-hydroxy-6-isopropoxy-quinoline-2-carbonyl)-amino]-acetic acid, [3-hydroxy-pyridine-2-carbonyl)-amino]-acetic acid, [N-((1-chloro-4-hydroxy-isoquinoline-3-carbonyl)-amino)-acetic acid, [(7-bromo-4-hydroxy-isoquinoline-3-carbonyl)-amino]-acetic acid, [(7-chloro-3-hydroxy-quinoline-2-carbonyl)-amino]-acetic acid, [(1-bromo-4-hydroxy-7-kifluoromethyl-isoquinoline-3-carbonyl)-amino]-acetic acid, [(1-Bromo-4-hydroxy-7-phenoxy-isoquinoline-3-carbonyl)-amino]-acetic acid, [(1-Chloro-4-hydroxy-7-phenoxy-isoquinoline-3-carbonyl)-amino]-acetic acid, [(1-Chloro-4-hydroxy-7-methoxy-isoquinoline-3-carbonyl)-amino]-acetic acid, [(1-chloro-4-hydroxy-isoquinoline-3-carbonyl)-amino]-acetic acid, [(4-Hydroxy-7-phenoxy-isoquinoline-3-carbonyl)-amino]-acetic acid, [(4-Hydroxy-7-phenylsulfanyl isoquinoline-3-carbonyl)-amino]-acetic acid, [(4-Hydroxy-6-phenylsulfanyl-isoquinoline-3-carbonyl)-amino]-acetic acid, 4-Oxo-1,4-dihydro-[1,10]phenanthroline-3-carboxylic acid, 4-hydroxy-5-methoxy-[1,10]phenanthroline-3-carboxylic acid ethyl ester, [(7-benzyloxy-1-chloro-4-hydroxy-isoquinoline-3-carbonyl)-amino]-acetic acid methyl ester, and 3-{[4-(3,3-Dibenzyl-ureido)-benzenesulfonyl]-[2-(4-methoxy-phenyl)-ethyl]-amino}-N-hydroxy-propionamide, are known HIF prolyl hydroxylase inhibitors.

One example of a family of HIF-1 modulating compounds that demonstrates increased pathogen killing by normal human cells in the present invention is mimosine and mimosine-like compounds. Mimosine, 3-Hydroxy-4-oxo-1(4H)-pyridinealanine, a plant amino acid and tyrosine analog, is a compound that chelates iron and inhibits mammalian DNA replication.

In addition, many growth factors and cytokines are known to stabilize HIF-1α under normoxic conditions, including insulin, insulin-like growth factor, epidermal growth factor, interleukin-1β (Zelzer et al., EMBO J 17:5085-94 (1998); Feldser et al., Cancer Res 59:3915-8 (1999); Richard et al., J Biol Chem 275:26765-71 (2000); Görlach et al., Circ Res 89:47-54 (2001); Haddad et al., FEBS Lett 505:269-74 (2001); Stiehl et al., FEBS Lett 512:15-62 (2002); Thornton et al., Biochem J 350 Pt 1, 307 (2000); Hellwig-Burgel et al., Blood 94, 1561 (1999); Sandau et al., Blood 97, 1009 (2001); Zhou et al., Am J Physiol Cell Physiol 284, C439 (2003); Zhou et al., Mol Biol Cell 14, 2216 (2003); Kasuno et al., J Biol Chem 279, 2550 (2004)) (each herein incorporated by reference). Similarly, NO and other certain reactive oxygen species are reported to stabilize HIF-1α under normoxia (Brune & Zhou, Curr Med Chem 10(10):845-55 (2003); Palmer et al., Mol Pharmacol 58:1197-203 (2000) (each herein incorporated by reference). Accordingly, such compounds could be utilized as potential lead compounds, to test under the invention assays or used to develop combinatorial libraries therefrom. HIF-1 or HIF-1 activity can also be modulated via proline hydroxylase inhibition. Such compounds and the generic structures to derive other suitable compounds are disclosed as follows:

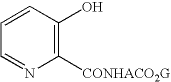

Preparation of 3-hydroxypyridine-2-carboxamides for treatment of fibrotic disease. Weidmann, Klaus; Baringhaus, Karl-heinz; Tschank, Georg; Bickel, Martin. (Hoechst A.-G., Germany). Eur. Pat. Appl. (1998), EP 900202 A1 (each herein incorporated by reference).

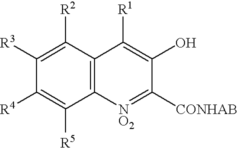

Use of hypoxia-inducible factor-α (HIF-α) stabilizers for enhancing erythropoiesis. Klaus, Stephen J.; Molineaux, Christopher J.; Neff, Thomas B.; Guenzler-Pukall, Volkmar; Lansetmo Parobok, Ingrid; Seeley, Todd W.; Stephenson, Robert C. (Fibrogen, Inc., USA). PCT hit. Appl. (2004), WO 2004108121 A1 (each herein incorporated by reference).

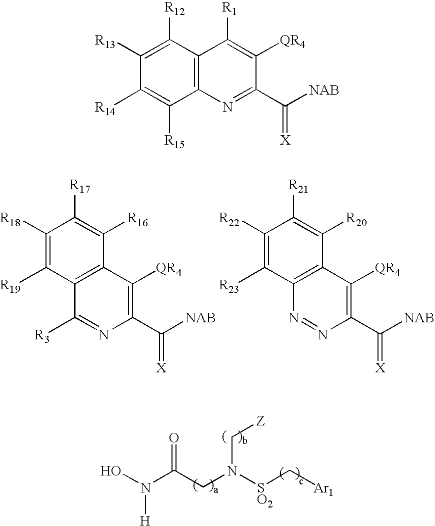

Preparation of substituted 3-hydroxyquinoline-2-carboxamides as prolyl-4-hydroxylase inhibitors. Weidmann, Klaus; Baringhaus, Karl-Heinz; Tschank, Georg; Bickel, Martin. (Hoechst A.-G., Germany; Fibrogen Inc.). Eur. Pat. Appl. (1997), EP 765871 A1 (each herein incorporated by reference).

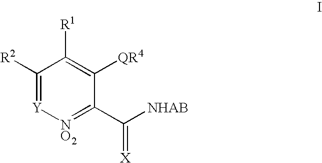

Pyridinecarboxamides and related compounds for treating fibrotic disease. Weidmann, Klaus; Baringhaus, Karl-Heinz; Tschank, Georg; Bickel, Martin. (Hoechst A.-G., Germany). Eur. Pat. Appl. (1995), EP 673929 A1 (each herein incorporated by reference).

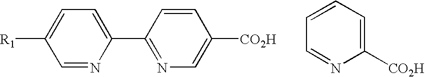

Novel inhibitors of prolyl 4-hydroxylase. 5. The intriguing structure-activity relationships seen with 2,2′-bipyridine and its 5,5′-dicarboxylic acid derivatives. Hales, Neil J.; Beattie, John F. Infect. Res. Dep., Zeneca Pharm., Macclesfield/Cheshire, UK. Journal of Medicinal Chemistry (1993), 36(24), 3853-8 (each herein incorporated by reference).

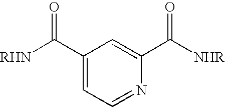

Beneficial effects of inhibitors of prolyl 4-hydroxylase in carbon tetrachloride-induced fibrosis of the liver in rats. Bickel, M.; Baader, E.; Brocks, D. G.; Engelbart, K.; Guenzler, V.; Schmidts, H. L.; Vogel, G. H. Hoechst A.-G., Frankfurt, Germany. Journal of Hepatology (1991), 13(Suppl. 3), S26-S34 (each herein incorporated by reference).

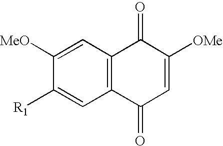

Inhibition of prolyl hydroxylase activity and collagen biosynthesis by fibrostatin C, a novel inhibitor produced by Streptomyces catenulae subsp. griseospora No. 23924. Ishimaru, Takenori; Kanamaru, Tsuneo; Takahashi, Toshiyuki; Okazaki, Hisayoshi. Cent. Res. Div., Takeda Chem. Ind., Ltd., Osaka, Japan. Journal of Antibiotics (1988), 41(11), 1668-74 (each herein incorporated by reference).

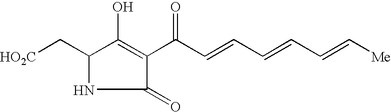

MBP039-06 as proline hydroxylase inhibitor and its manufacture with Phaeosphaeria. Furui, Megumi; Takashima, Junko; Sudo, Keiko; Chiba, Noriko; Mikawa, Takashi. (Mitsubishi Chemical Industries Co., Ltd., Japan). Jpn. Kokai Tokkyo Koho (1993), JP 05239023 A2 19930917 (each herein incorporated by reference).

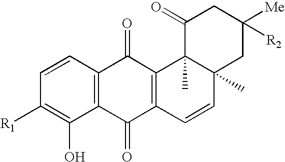

The absolute configuration of P-1894B, A potent prolyl hydroxylase inhibitor. Ohta, Kazuhiko; Mizuta, Eiji; Okazaki, Hisayoshi; Kishi, Toyokazu. Cent. Res. Div., Takeda Chem. Ind., Ltd., Osaka, Japan. Chemical & Pharmaceutical Bulletin (1984), 32(11), 4350-9 (each herein incorporated by reference).

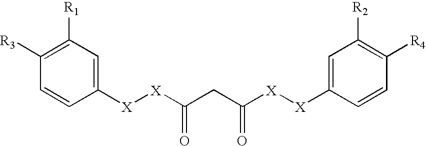

Preparation of Novel Curcumin/Tetrahydrocurcumin Derivatives for Use in cosmetics, pharmaceuticals and for nutrition. Rieks, Andre; Kaehler, Markus; Kirchner, Ulrike; Wiggenhorn, Kerstin; Kinzer, Mona. (Andre Rieks—Labor fuer Enzymtechnologie G.m.b.h., Germany). WO 04/031122 (each herein incorporated by reference).

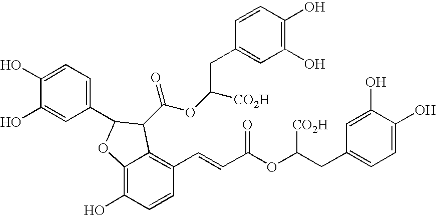

Review on pharmacology in lithospermic acid B. Peng, Zonggen; Chen, Hongshan. Department of Virology, Institute of Medicinal Biotechnology, Chinese Academy of Medical Sciences and Peking Union Medical College, Beijing, Peop. Rep. China. Zhongguo Yaoxue Zazhi (Beijing, China) (2003), 38(10), 744-747 (each herein incorporated by reference).

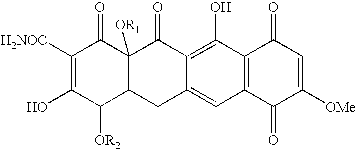

Proline hydroxylase-inhibiting tetracyclines and their manufacture with Streptomyces species. Furui, Megumi; Takashima, Junko; Sudo, Keiko; Chiba, Noriko; Sashita, Reiko. (Mitsubishi Chemical Industries Co., Ltd., Japan). Jpn. Kokai Tokkyo Koho (1994), JP 06339395 A2 (each herein incorporated by reference).

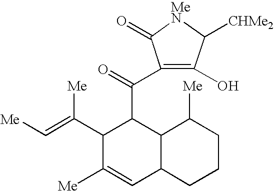

A novel proline hydroxylase inhibitor MBP049-13 and its manufacture with Ophiobolus. Furui, Megumi; Takashima, Junko; Mikawa, Takashi; Yoshikawa, Nobuji; Ogishi, Haruyuki. (Mitsubishi Kasei K. K., Japan). Jpn. Kokai Tokkyo Koho (1992), JP 04074163 A2 (each herein incorporated by reference).

Fast and efficient analytical techniques are advantageous for using the complicated product mixtures and detecting by-products. For example, a combinatorial chemical library such as a polypeptide library is formed by combining a set of chemical building blocks (e.g., in one example, amino acids) in every possible way for a given compound length (i.e., the number of amino acids in a polypeptide compound of a set length). Exemplary libraries include peptide libraries, non-peptide oligomer, nucleic acid libraries, antibody libraries (see, e.g., Vaughn et al., Nature Biotechnology, 14(3):309-314 (1996) and PCT/US96/10287), carbohydrate libraries (see, e.g., Liang et al., Science 274:1520-1522 (1996) and U.S. Pat. No. 5,593,853), peptide nucleic acid libraries (see, e.g., U.S. Pat. No. 5,539,083), and small organic molecule libraries (see, e.g., benzodiazepines, Baum C&EN January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337) and the like (Lam, K. S., Anticancer Drug Des. 12:145 (1997)) (each herein incorporated by reference).

Preparation and screening of combinatorial or other libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991); and Houghton et al., Nature 354:84-88 (1991)). Other chemistries for generating chemical diversity libraries can also be used. Other exemplary methods for the synthesis of molecular libraries can be found in the art, for example in: Erb et al. 1994. Proc. Natl. Acad. Sci. USA 91:11422; Horwell et al., Immunopharmacology 33:68 (1996); and in Gallop et al., J. Med. Chem. 37:1233(1994) (each herein incorporated by reference).

Libraries of compounds may be presented in solution (e.g., Houghten (1992) Biotechniques 13:412-421), or on beads (Lam, Nature 354:82-84 (1991)), chips (Fodor, Nature 364:555-556 (1993)), bacteria (Ladner U.S. Pat. No. 5,223,409), spores (Ladner U.S. Pat. No. '409), plasmids (Cull et al., Proc Natl Acad Sci USA 89:1865-1869 (1992)) or on phage (Scott and Smith, Science 249:386-390 (1990)); (Devlin, Science 249:404-406 (1990)); (Cwirla et al., Proc. Natl. Acad. Sci. 87:6378-6382 (1990)); (Felici, J. Mol. Biol. 222:301-310 (1991)); (Ladner supra.) (each herein incorporated by reference). Other types of peptide libraries may also be expressed see, for example, U.S. Pat. Nos. 5,270,181 and 5,292,646) (each herein incorporated by reference). In still another embodiment, combinatorial polypeptides can be produced from a cDNA library.

In other embodiments, the compounds can be nucleic acid molecules. In preferred embodiments, nucleic acid molecules for testing are small oligonucleotides. Such oligonucleotides can be randomly generated libraries of oligonucleotides or can be specifically designed to reduce the activity of HIF-1. For example, in one embodiment, these oligonucleotides are sense or antisense oligonucleotides. Methods of designing such oligonucleotides given the sequences of a particular transcription factor polypeptide, such as a HIF-1α polypeptide, are within the skill of the art.

More generally, screening methods according to the invention will concentrate in the early stages on finding candidate compounds, initially by screening libraries of between 30,000 to 50,000 compounds. Once candidates have been identified, subsequent stages involve the verification of the structures of these compounds, for example, using techniques known to those of skill in the art such as High Performance Liquid Chromatography (HPLC)/Mass Spectrometry for dissolved stock and HPLC/Mass Spectrometry and Nuclear magnetic resonance Spectroscopy for solid stock (see Mass Spectrometry in Drug Discovery; Rossi & Sinz (Eds) ISBN: 0824706072; Nuclear Magnetic Resonance Spectroscopy, Nelson). HPLC analysis can be readily used to identify quantitatively the above described reaction products, using, e.g., tritiated substrates, and the like. Similarly, on a more qualitative level, thin layer chromatography (TLC) can also be used to identify reaction products. Subsequent follow-up studies will concentrate on evaluating the efficacy of the compounds, generating IC50 values (1-30 μM) from high throughput enzyme inhibition assays (such as using scintillation proximity assays (available from Amersham Pharmacia Biotech) or equivalent techniques). If available, in silico pharmacokinetics studies may be used to expedite the screening process (suitable Bioinformatics expertise may be sought from companies such as Inpharmatica Ltd, London and De Novo Ltd, Cambridge).

Hit to lead optimisation and validation involves evaluating a selection (approximately 200) of the best candidates which yielded 1 μM potency (IC50=1 μM) in a high throughput enzyme inhibition assay. Other suitable techniques will involve the determination of the specific absorption rate (SAR) by CaCo 2 intestinal cell absorption assay (MultiScreen CaCo-2 Assay system available from Millipore; BD BioCoat HTS CaCo-2 assay system available from BD Biosciences), the determination of the selectivity of the candidate compounds (against related family members) and evaluating the reversibility and kinetics of binding of these compounds. The studies may also involve the measurement of the in vitro metabolic stability of the candidate compounds in primary human and rat liver microsomes; measurement of the in vitro inhibition and induction of human and mammalian cytochrome P450s; the determination of in vitro toxicity (such as by using MTT assays [available from Roche] and LDH assays [available from Cambridge Bioscience]).

Lead optimization and validation then takes compounds that satisfy the following criteria for functional assay and in vivo pharmacokinetics studies: 1 nM potency in a high throughput enzyme inhibition assay; solubility of about 0.1 mg/ml on lead compounds; functionality in the presence of human serum albumin (HSA) and/or rodent serum albumin (RSA); 1000× selectivity against family members; demonstrated in vitro absorption in CaCo2 intestinal cells; demonstrated in vitro metabolic stability in primary human and rat liver S9 microsomes; profiles of human and maminalian cytochrome P450 inhibition/induction having been determined (a variety of C14-labeled substrates are available for P450 assays from Amersham Biosciences); mechanism of interaction with target having been determined; the most advantageous pharmacokinetics specificity profile. This latter test may involve a toxicity test for drugs, such as the Human Ether-a-go-go gene (HERG) potassium channel assay that is a cardiotoxicity test (Compton et al., (1996) Circulation. 94(5): 1018-22), the FLIPR Membrane Potential assay test (kit available from Molecular Devices Ltd) or alternative in vitro mutagenicity tests (such as the Ames test [Benedict et al., (1977) Cancer Res. 37:2209-13]; kit available from Litron Laboratories] or the clastogenicity assay [Sister Chromatid Exchange assay kit is available from Litron Laboratories]).

In addition, as noted, compound screening equipment for high-throughput screening is generally available, e.g., using any of a number of well known robotic systems that have also been developed for solution phase chemistries useful in assay systems. These systems include automated workstations including an automated synthesis apparatus and robotic systems utilizing robotic arms. Any of the above devices are suitable for use with the present invention, e.g., for high-throughput screening of potential modulators. The nature and implementation of modifications to these devices (if any) so that they can operate as discussed herein will be apparent to persons skilled in the relevant art.

Indeed, entire high throughput screening systems are commercially available. These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. Similarly, microfluidic implementations of screening are also commercially available.

The manufacturers of such systems provide detailed protocols of the various high throughputs. Thus, for example, Zymark Corp. provides technical bulletins describing screening systems for detecting the modulation of gene transcription, ligand binding, and the like. The integrated systems herein, in addition to providing for sequence alignment and, optionally, synthesis of relevant nucleic acids, can include such screening apparatus to identify modulators that have an effect on one or more polynucleotides or polypeptides according to the present invention.

Functional (cell-based) assays are then developed to validate the lead compounds. Further studies of lead compound development may involve an investigation of in vivo pharmacokinetics data in animal experiments; in vivo positive pharmacokinetics results correlated with in vitro data and in silico data; in vivo activity in a functional animal model; in vivo safety studies—central nervous system (CNS)/cardiovascular (CVS) toxicity profiles should be determined. In one aspect of the present invention, there are provided.

Another aspect of this invention provides for any screening kits that are based or developed from any of the aforementioned assays.

In yet another embodiment, computer programs can be used to identify individual compounds or classes of compounds with an increased likelihood of modulating HIF-1 polypeptide activity. Such programs can screen for compounds with the proper molecular and chemical complementarities with a chosen transcription factor. In this manner, the efficiency of screening for transcription factor modulating compounds in the assays described above can be enhanced.

### 3. Structure Based Drug Design

The invention also pertains, at least in part, to a computational screening of small molecule databases for chemical entities or compounds that can bind in whole, or in part, to HIF-1. In this screening, the quality of fit of such entities or compounds to the binding site may be judged either by shape complementarity or by estimated interaction energy (Meng, E. C. et al., 1992, J. Coma. Chem., 13:505-524). Such a procedure allows for the screening of a very large library of potential transcription factor modulating compounds for the proper molecular and chemical complementarities with a selected protein or class or proteins.

HIF-1 modulating compounds identified through computational screening can later be passed through the in vivo assays described herein as further screens. For example, HIF-1 agonists or HIF-1 interacting protein inhibiting compound identified through computational screening could be tested for its ability to promote HIF-1 stability or accumulation in a cell system containing a counterselectable marker under the control of a HRE. The promotion of HIF-1 activity in the foregoing assay would be indicative of a compound that could be identified as a compound that reduces or eradicate infection. Other suitable assays are known in the art.

The design of compounds that bind to, modulate, or inhibit transcription factors, generally involves consideration of two factors. First, the compound must be capable of physically and structurally associating with a particular transcription factor. Noncovalent molecular interactions important in the association of a transcription factor with a modulating compound include hydrogen bonding, van der Waals and hydrophobic interactions.

Second, the modulating compound must be able to assume a conformation that allows it to associate with the selected transcription factor. Although certain portions of the inhibiting compound will not directly participate in this association with the transcription factor, those portions may still influence the overall conformation of the molecule. This, in turn, may have a significant impact on potency. Such conformational requirements include the overall three dimensional structure and orientation of the chemical entity or compound in relation to all or a portion of the binding site, e.g., active site or accessory binding site of HIF-1, or the spacing between functional groups of a compound comprising several chemical entities that directly interact with the particular transcription factor.

In a further embodiment, the potential modulating effect of a chemical compound on HIF-1 is analyzed prior to its actual synthesis and testing by the use of computer modeling techniques. If the theoretical structure of the given compound suggests insufficient interaction and association between it and the selected transcription factor, synthesis and testing of the compound is avoided. However, if computer modeling indicates a strong interaction, the molecule may then be synthesized and tested for its ability to bind to the selected transcription factor and modulate the transcription factor's activity.

A HIF-1 modulating compound or other (e.g., HIF-1 interacting protein, HRE, and the like) binding compound may be computationally evaluated and designed by screening and selecting chemical entities or fragments for their ability to associate with the individual small molecule binding sites or other areas of a transcription factor.

One skilled in the art may use one of several methods to screen chemical entities or fragments for their ability to associate with a selected factor and more particularly with the individual small molecule binding sites of the particular factor. This process may begin by visually inspecting the structure of the transcription factor on a computer screen based on the atomic coordinates of the transcription factor crystals. Selected chemical entities may then be positioned in a variety of orientations, or docked, within an individual binding site of the transcription factor. Docking may be performed using software such as Quanta and Sybyl, followed by energy minimization with standard molecular mechanics forcefields or dynamics with programs such as CHARMM (Brooks, B. R. et al., 1983, J. Comp. Chem., 4:187-217) or AMBER (Weiner, S. J. et al., 1984, J. Am. Chem. Soc., 106:765-784).

Specialized computer programs may also assist in the process of selecting molecules that bind to HIF-1. The programs include, but are not limited to:

1. GRID (Goodford, P. J., 1985, “A Computational Procedure for Determining Energetically Favorable Binding Sites on Biologically Important Macromolecules” J. Med. Chem., 28:849-857 GRID is available from Oxford University, Oxford, UK.
2. AUTODOCK (Goodsell, D. S. and A. J. Olsen, 1990, “Automated Docking of Substrates to Proteins by Simulated Annealing” Proteins: Structure. Function, and Genetics, 8:195-202. AUTODOCK is available from Scripps Research Institute, La Jolla, Calif. AUTODOCK helps in docking inhibiting compounds to a selected transcription factor in a flexible manner using a Monte Carlo simulated annealing approach. The procedure enables a search without bias introduced by the researcher.
3. MCSS (Miranker, A. and M. Karplus, 1991, “Functionality Maps of Binding Sites: A Multiple Copy Simultaneous Search Method.” Proteins: Structure, Function and Genetics, 11:29-34). MCSS is available from Molecular Simulations, Burlington, Mass.
4. MACCS3D (Martin, Y. C., 1992, J. Med. Chem., 35:2145-2154) is a 3D database system available from MDL Information Systems, San Leandro, Calif.
5. DOCK (Kuntz, I. D. et al., 1982, “A Geometric Approach to Macromolecule-Ligand Interactions” J. Mol. Biol., 161:269-288). DOCK is available from University of California, San Francisco, Calif. DOCK is based on a description of the negative image of a space-filling representation of the molecule (i.e. the selected transcription factor) that should be filled by the inhibiting compound. DOCK includes a force-field for energy evaluation, limited conformational flexibility and consideration of hydrophobicity in the energy evaluation.
6. MCDLNG (Monte Carlo De Novo Ligand Generator) (D. K. Gehlhaar, et al. 1995. J. Med. Chem. 38:466-472). MCDLNG starts with a structure (i.e. anX-ray crystal structure) and fills the binding site with a close packed array of generic atoms. A Monte Carlo procedure is then used to randomly: rotate, move, change bond type, change atom type, make atoms appear, make bonds appear, make atoms disappear, make bonds disappear, etc. The energy function used by MCDLNG favors the formation of rings and certain bonding arrangements. Desolvation penalties are given for heteroatoms, but heteroatoms can benefit from hydrogen bonding with the binding site.

### 7. MCELL (Coggen, J. S. et al., 2005. Science 446-451) A Monte Carlo Simulator of Cellular Microphysiology.

Useful programs to aid one of skill in the art in connecting the individual chemical fragments include:

1. 3D Database systems such as MACCS3D (MDL Information Systems, San Leandro, Calif. This area is reviewed in Martin, Y. C., 1992, “3D Database Searching in Drug Design”, J. Med. Chem., 35, pp. 2145-2154).
2. CAVEAT (Bartlett, P. A. et al., 1989, “CAVEAT: A Program to Facilitate the Structure-Derived Design of Biologically Active Molecules”. In Molecular Recognition in Chemical and Biological Problems”, Special Pub., Royal Chem. Soc., 78, pp. 182-196). CAVEAT is available from the University of California, Berkeley, Calif.
3. HOOK (available from Molecular Simulations, Burlington, Mass.). HOOK proposes docking sites by using multiple copies of functional groups in simultaneous searches.

In another embodiment, transcription factor modulating compounds may be designed as a whole or “de novo” using either an empty active site or optionally including some portion(s) of a known inhibiting compound(s). These methods include:

1. LUDI (Bohm, H. J., “The Computer Program LUDI: A New Method for the De Novo Design of Enzyme Inhibiting compounds”, J. Com R. Aid. Molec. Design, 6, pp. 61-78 (1992)). LUDI is available from Biosym Technologies, San Diego, Calif. LUDI is a program based on fragments rather than on descriptors. LUDI proposes somewhat larger fragments to match with the interaction sites of a macromolecule and scores its hits based on geometric criteria taken from the Cambridge Structural Database (CSD), the Protein Data Bank (PDB) and on criteria based on binding data. LUDI is a library based method for docking fragments onto a binding site. Fragments are aligned with 4 directional interaction sites (lipophilic-aliphatic, lipophilicaromatic, hydrogen donor, and hydrogen acceptor) and scored for their degree of overlap. Fragments are then connected (i.e. a linker of the proper length is attached to each terminal atom in the fragments). Note that conformational flexibility can be accounted for only by including multiple conformations of a particular fragment in the library. 2. LEGEND (Nishibata, Y. and A. Itai, Tetrahedron, 47, p. 8985 (1991)). LEGEND is available from Molecular Simulations, Burlington, Mass. 3. CoMFA (Conformational Molecular Field Analysis) (J. J. Kaminski. 1994. Adv. Drug Delivery Reviews 14:331-337.) CoMFA defines 3-dimensional molecular shape descriptors to represent properties such as hydrophobic regions, sterics, and electrostatics. Compounds from a database are then overlaid on the 3D pharmacophore model and rated for their degree of overlap. Small molecule databased that be searched include: ACD (over 1,000,000 compounds), Maybridge (about 500,000 compounds), NCl (about 500,000 compounds), and CCSD. In measuring the goodness of the fit, molecules can either be fit to the 3D molecular shape descriptors or to the active conformation of a known inhibiting compound. 4. LeapFrog (available from Tripos Associates, St. Louis, Mo.).

FlexX (©1993-2002 GMD German National Research Center for Information Technology; Rarey, M. et al., J. Mol. Biol., 261:407-489) is a fast, flexible docking method that uses an incremental construction algorithm to place ligands into and active site of the transcription factor. Ligands (e.g., transcription factor modulating compounds) that are capable of “fitting” into the active site are then scored according to any number of available scoring schemes to determine the quality of the complimentarily between the active site and ligand.

Other molecular modeling techniques may also be employed in accordance with this invention. See, e.g., Cohen, N. C. et al., “Molecular Modeling Software and Methods for Medicinal Chemistry, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Candidate transcription factor modulating compounds can be evaluated for their modulating, e.g., inhibitory or stimulatory, activity using conventional techniques which may involve determining the location and binding proximity of a given moiety, the occupied space of a bound inhibiting compound, the deformation energy of binding of a given compound and electrostatic interaction energies. Examples of conventional techniques useful in the above evaluations include, but are not limited to, quantum mechanics, molecular dynamics, Monte Carlo sampling, systematic searches and distance geometry methods (Marshall, G. R., 1987, Ann. Ref Pharmacol. Toxicol, 27:193). Examples of computer programs for such uses include, but are not limited to, Gaussian 92, revision E2 (Gaussian, Inc. Pittsburgh, Pa.), AMBER version 4.0 (University of California, San Francisco), QUANTA/CHARMM (Molecular Simulations, Inc., Burlington, Mass.), and Insight IL/Discover (Biosym Technologies Inc., San Diego, Calif.). These programs may be implemented, for example, using a Silicon Graphics Indigo2 workstation or IBM RISC/6000 workstation model 550. Other hardware systems and software packages will be known and of evident applicability to those skilled in the art.

Once a compound has been designed and selected by the above methods, the efficiency with which that compound may bind to a particular transcription factor may be tested and optimized by computational evaluation. An effective transcription factor modulating compound should demonstrate a relatively small difference in energy between its bound and free states (i.e., a small deformation energy of binding). Transcription factor modulating compounds may interact with the selected transcription factor in more than one conformation that is similar in overall binding energy. In those cases, the deformation energy of binding may be taken to be the difference between the energy of the free compound and the average energy of the conformations observed when the inhibiting compound binds to the enzyme.

A compound designed or selected as interacting with HIF-1 may be further computationally optimized so that in its bound state it would preferably lack repulsive electrostatic interaction with the target protein. Such non-complementary (e.g., electrostatic) interactions include repulsive charge-charge, dipole-dipole and charge-dipole interactions. Specifically, the sum of all electrostatic interactions between the modulating compound and the enzyme when the modulating compound is bound to the selected transcription factor, preferably make a neutral or favorable contribution to the enthalpy of binding.

Specific computer software is available in the art to evaluate compound deformation energy and electrostatic interaction. Examples of programs designed for such uses include: Gaussian 92, revision C [M. J. Frisch, Gaussian, Inc., Pittsburgh, Pa. ©1992]; AMBER, version 4.0 [P. A. Kollman, University of California at San Francisco, ©1994]; QUANTA/CHARMM [Molecular Simulations, Inc., Burlington, Mass. ©1994]; and Insight II/Discover (Biosysm Technologies Inc., San Diego, Calif. ©1994). These programs may be implemented, for instance, using a Silicon Graphics workstation, IRIS 4D/35 or IBM RISC/6000 workstation model 550. Other hardware systems and software packages will be known to those skilled in the art.

Once HIF-1 modulating compound has been optimally selected or designed, as described above, substitutions may then be made in some of its atoms or side groups in order to improve or modify its binding properties. Initial substitutions are preferable conservative, i.e., the replacement group will have approximately the same size, shape, hydrophobicity and charge as the original group. Substitutions known in the art to alter conformation should be avoided. Such substituted chemical compounds may then be analyzed for efficiency of fit to the selected transcription factor by the same computer methods described above.

Computer programs can be used to identify unoccupied (aqueous) space between the van der Waals surface of a compound and the surface defined by residues in the binding site. These gaps in atom-atom contact represent volume that could be occupied by new functional groups on a modified version of the lead compound. More efficient use of the unoccupied space in the binding site could lead to a stronger binding compound If the overall energy of such a change is favorable. A region of the binding pocket which has unoccupied volume large enough to accommodate the volume of a group equal to or larger than a covalently bonded carbon atom can be identified as a promising position for functional group substitution. Functional group substitution at this region can constitute substituting something other than a carbon atom, such as oxygen. If the volume is large enough to accommodate a group larger than a carbon atom, a different functional group which would have a high likelihood of interacting with protein residues in this region may be chosen. Features which contribute to interaction with protein residues and identification of promising substitutions include hydrophobicity, size, rigidity and polarity. The combination of docking, Kᵢ estimation, and visual representation of sterically allowed room for improvement permits prediction of potent derivatives.

Once HIF-1 modulating compound has been selected or designed, computational methods to assess its overall likeness or similarity to other molecules can be used to search for additional compounds with similar biochemical behavior. In such a way, for instance, HTS derived hits can be tested to assure that they are bona fide ligands against a particular active site, and to eliminate the possibility that a particular hit is an artifact of the screening process. There are currently several methods and approaches to determine a particular compound's similarity to members of a virtual database of compounds. One example is the OPTISIM methodology that is distributed in the Tripos package, SYBYL (© 1991-2005 Tripos, Inc., St. Louis, Mo.). OPTISIM exploits the fact that each 3-dimensional representation of a molecule can be broken down into a set of 2-dimensional fragments and encoded into a pre-defined binary string. The result is that each compound within a particular set is represented by a unique numerical code or fingerprint that is amenable to mathematical manipulations such as sorting and comparison. OPTISIM is automated to calculate and report the percent difference in the fingerprints of the respective compounds for instance according to the using a formalism known as the Tanimoto coefficient. For instance, a compound that is similar in structure to another will share a high coefficient. Large virtual databases of commercially available compounds or of hypothetical compounds can be quickly screened to identify compounds with high Tanimoto coefficient.

Once a series of similar transcription factor modulating compounds has been identified and expanded by the methods described, their experimentally determined biological activities can be correlated with their structural features using a number of available statistical packages. In a typical project within the industry, the CoMFA (COmparative Molecular Field Analysis) and QSAR (Quantitative Structure Activity Relationship) packages within the SYBYL suite of programs (Tripos Associates, St. Louis, Mo.) are utilized. In CoMFA, a particular series of compounds with measured activities are co-aligned in a manner that is believed to emulate their arrangement as they interact with the active site. A 3-dimensional lattice, or grid is then constructed to encompass the collection of the so-aligned compounds. At each point on the lattice, an evaluation of the potential energy is determined and tabulated-typically potentials that simulate the electronic and steric fields are determined, but other potential functions are available. Using the statistical methods such as PLS (Partial Least Squares), correlation between the measured activities and the potential energy values at the grid-points can be determined and summed in a linear equation to produce the overall molecular correlation or QSAR model. A particularly useful feature in CoMFA is that the individual contribution for each grid-point is known; the importance of the grid points upon the overall correlation can be visualized graphically in what is referred to as a CoMFA field. When this field is combined with the original compound alignment, it becomes a powerful tool to rationalize the activities of the individual compounds from whence the model was derived, and to predict how chemical modification of a reference compound would be effected. As an example, a QSAR model was developed for a set of 92 benzodiazepines using the method described above.

Structure based drug design as described herein or known in the art can be used to identify candidate compounds or to optimize compounds identified in screening assays described herein.

The invention pertains, per se, to not only the methods for identifying the transcription factor modulating compounds, but to the compounds identified by the methods of the invention as well as methods for using the identified compounds.

### VII. Pharmaceutical Compositions

The agents which modulate the activity or expression of transcription factors can be administered to a subject directly or using pharmaceutical compositions suitable for such administration. Such compositions typically comprise the agent of interest (e.g., nucleic acid molecule, protein, or antibody) and a pharmaceutically acceptable carrier.

In one embodiment, such compositions can be administered in combination with a second agent. For example, an agent that modulates the activity or expression of HIF-1 can be administered to a subject along with a second agent that is effective at controlling the growth or virulence of a microbe. Exemplary agents include antibiotics or biocides. Such a second agent can be administered or contacted with a microbe or a surface either separately or as part of the pharmaceutical composition comprising the agent which modulates the activity or expression of the transcription factor.

As used herein the language “pharmaceutically acceptable carrier” is intended to include any and all solvents, dispersion media, coatings, antibacterial and antifungal agents, isotonic and absorption delaying agents, anti-inflammatory, stabilizers, and the like, compatible with pharmaceutical administration. The use of such media and agents for pharmaceutically active substances is well known in the art. The indication to be treated, along with the physical, chemical, and biological properties of the drug, dictate the type of formulation and the route of administration to be used, as well as whether local or systemic delivery would be preferred. Except insofar as any conventional media or agent is incompatible with the active compound, use thereof in the compositions is contemplated. Supplementary active compounds can also be incorporated into the compositions. Carrier molecules may be genes, polypeptides, antibodies, liposomes or indeed any other agent provided that the carrier does not itself induce toxicity effects or cause the production of antibodies that are harmful to the individual receiving the pharmaceutical composition. Further examples of known carriers include polysaccharides, polylactic acids, polyglycolic acids and inactive virus particles. Carriers may also include pharmaceutically acceptable salts such as mineral acid salts (for example, hydrochlorides, hydrobromides, phosphates, sulphates) or the salts of organic acids (for example, acetates, propionates, malonates, benzoates). Pharmaceutically acceptable carriers may additionally contain liquids such as water, saline, glycerol, ethanol or auxiliary substances such as wetting or emulsifying agents, pH buffering substances and the like. Carriers may enable the pharmaceutical compositions to be formulated into tablets, pills, dragees, capsules, liquids, gels, syrups, slurries, suspensions to aid intake by the patient. Various formulations and drug delivery systems are available in the art, and a thorough discussion of pharmaceutically acceptable carriers are available in the art (see, e.g., USIP. Remington; The Science and Practice of Pharmacology (Lippincott Williams & Wilkins, 21st ed. 2005); and Ansel & Stoklosa, Pharmaceutical Calculations (Lippincott Williams & Wilkins, 11th ed., 2001)

A pharmaceutical composition used in the therapeutic methods of the invention is formulated to be compatible with its intended route of administration. Examples of routes of administration include parenteral, e.g., intravenous or intra-arterial, intradermal, subcutaneous, oral (e.g., inhalation), transdermal (topical), transmucosal, nasal, pulmonary, ocular, gastrointestinal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Alternate routes of administration include intraperitoneal, intra-articular, intracardiac, intracisternal, intradermal, intralesional, intraocular, intrapleural, intrathecal, intrauterine, intraventricular, and the like.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks, or presented in the form of implants and pumps requiring incision. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, emulsions and the like. The compounds may also be formulated in rectal compositions, such as suppositories or retention enemas.

Preferably, pharmaceutical compositions suitable for injectable use include sterile aqueous solutions (where water soluble) or dispersions and sterile powders for the extemporaneous preparation of sterile injectable solutions or dispersion. Depending on the injection site, the vehicle may contain water, synthetic or vegetable oil, and/or organic co-solvents. In certain instances, such as with lyophilized product or a concentrate, the parenteral formulation would be reconstituted or diluted prior to administration. Depot formulations, providing controlled or sustained release of an invention compound, may include injectable suspensions of nano/micro particles or nano/micro or non-micronized crystals. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, poly(ol) (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates the expression and/or activity of a transcription in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; dissolution retardant; anti-adherants; cationic exchange resin; wetting agents; antioxidants; preservatives; a disintegrating agent such as alginic acid, Primogel, or corn starch; a lubricant such as magnesium stearate or Sterotes; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient.

The agents that modulate the activity of transcription factors can also be prepared in the form of suppositories (e.g., with conventional suppository bases such as cocoa butter and other glycerides) or retention enemas for rectal delivery.

In one embodiment, the agents that modulate transcription factor expression and/or activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates the expression and/or activity of HIF-1 and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population).

Preliminary in vitro cytotoxicity (Tox) assays of all newly synthesized HIF-1 modulators can be performed on African green monkey kidney COS-1 and Chinese hamster ovary (CHOK1) cell lines according to standard methods and in a relatively high-throughput manner using automatic liquid dispensers and robotic instrumentation. Briefly, cell cultures are washed, trypsinized, and harvested. The cell suspensions are then prepared, used to seed 96-well blackwalled microtiter plates, and incubated under tissue culture conditions overnight at 37.degree. C. On the following day, serial dilutions of a HIF-1 modulators are transferred to the plates that are then incubated for a period of 24 hr. Subsequently, the media/drug is aspirated and 50 μl of Resazurin is added. Resazurin is a soluble nontoxic dye that is used as an indicator of cellular metabolism and is routinely employed for these types of cytotoxicity assays.

Plates are then incubated under tissue culture conditions for 2 hr and then in the dark for an additional 30 min. Fluorescence measurements (excitation 535 nm, emission 590 nm) are recorded and are used to calculate toxicity versus control cells. Ultimately, Tox50 and Tox100 values will be determined and these values represent the concentration of compound necessary to inhibit cellular proliferation by 50% and 100%, respectively. Control cytotoxic and noncytotoxic compounds are routinely included in all assays. The goal of these experiments is to identify compounds with little or no measurable in vitro cytotoxicity.

HIF-1 inhibitors that perform favorably in the cellular Tox assays will be studied in a mouse model of acute toxicity. Briefly, groups of female CD1 mice (n=5) will be treated with the test compound or a control compound (vehicle) via a subcutaneous route of administration at up to three dose levels for five days. Overt signs of animal distress, e.g., general clinical observations, weight loss, feed consumption, etc., will be monitored daily. Upon completion of the study and hematological and pathological tissue analyses and serum chemistries can be performed. The goal will be to identify compounds without detectable acute toxicity.

The dose ratio between toxic and therapeutic effects is the therapeutic index and can be expressed as the ratio LD50/ED50. Agents that exhibit high therapeutic indices are preferred. While agents that exhibit toxic side effects may be used, care should be taken to design a delivery system that targets such agents to the site of affected tissue in order to minimize potential damage to uninfected cells and, thereby, reduce side effects.

The amount of the compound in the composition should also be in therapeutically effective amounts. The phrase “therapeutically effective amounts” used herein refers to the amount of agent needed to treat, ameliorate, or prevent (for example, when used as a vaccine) a targeted disease or condition. An effective initial method to determine a “therapeutically effective amount” may be by carrying out cell culture assays (for example, using neoplastic cells) or using animal models (for example, mice, rabbits, dogs or pigs). In addition to determining the appropriate concentration range for an invention composition to be therapeutically effective, animal models may also yield other relevant information such as preferable routes of administration that will give maximum effectiveness. Such information may be useful as a basis for patient administration. A “patient” as used in herein refers to the subject who is receiving treatment by administration of the compound of interest. Preferably, the patient is human, but the term may also include animals.

The data obtained from the cell culture assays and animal studies can be used in formulating a range of dosage for use in humans. The dosage of such transcription factor modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of a protein, polypeptide, or antibody can include a single treatment or, preferably, can include a series of treatments. It will also be appreciated that the effective dosage of antibody, protein, or polypeptide used for treatment may increase or decrease over the course of a particular treatment. Changes in dosage may result and become apparent from the results of diagnostic assays as described herein. The therapeutically-effective dosage will generally be dependent on the patient's status at the time of administration. The precise amount can be determined by routine experimentation but may ultimately lie with the judgment of the clinician.

The present invention encompasses agents which modulate expression and/or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression and/or activity to be modulated. Such appropriate doses may be determined using the assays described herein. When one or more of these small molecules is to be administered to an animal (e.g., a human) in order to modulate expression and/or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the general health, age, body weight, general health, gender, and diet of the subject, the time and frequency of administration, the route of administration, the rate of excretion, any drug combination, and the degree of expression and/or activity to be modulated, the severity of the disease state in the patient, reaction sensitivities and the patient's tolerance or response to the therapy.

### Methods of Treatment

Therapies to treat infection may be based upon (1) administration of normal HIF-1 proteins or HIF-1 interacting proteins, (2) gene therapy with normal HIF-1 or HIF-1 interacting genes, (3) biological therapy which enhance or potentiate HIF-activity/level or alternately, antagonize HIF-1 inhibitors, (4) immunotherapy based upon antibodies to potentiate the activity of HIF-1, or (5) small molecules (drugs) which alter HIF-1 (e.g., increase) or HIF-1 interacting protein (e.g., decrease HIF-1 inhibitors or degrading factors, or alternatively, increase HIF-1 potentiating factors) expression or activity.

The present invention provides for both prophylactic and therapeutic methods of treating a subject, e.g., a human, at risk of (or susceptible to) or having a microbial infection by administering an agent which modulates the expression, concentration and/or activity of a HIF-1. The term “treatment” with respect to infections, is contemplated as the application or administration of a therapeutic agent to a patient, who has an infection, a symptom of an infection, or a predisposition toward an infection, with the purpose to cure, heal, alleviate, relieve, alter, remedy, ameliorate, improve or affect the infection, the symptoms of the infection, or the predisposition toward an infection, e.g., a pathogenic infection. With respect to the terms “activation”, “increasing”, “strengthening” or “enhancing” (or such other similar terms) of the “innate immune system” are meant improvements of all kinds of situations, where the immune system of a person is supposed to achieve a higher degree of performance including strengthening of the immune system of said person; e.g., reduction, prevention and ameliorating of the period and intensity of infections.

In one embodiment, the invention provides for a method of treatment, either prophylactic or therapeutic of a subject or a patient population at risk of infection, e.g., individuals in long term care facilities, nosocomial infections, critical and intensive care units, transplant (kidney) services, pre- or post-surgical units, (urologic) or oncology units, sexually active young females, postmenopausal women that experience recurrent UTI, individuals working under unsanitary conditions or in military environments, and the like. In addition, the subject methods and compounds can be used in the prophylactic treatment of asymptomatic bacteriuria in pregnant women and patients undergoing urologic surgery or renal transplants. Immunocompromised or catheterized patients could also be treated using the subject methods and compounds. Alternatively, the present invention provides for enhancing the immune response to prevent, fight, reduce, ameliorate, or reduce the period and intensity of transmissible diseases, such as respiratory tract infections as the common cold or influenza (flu).

In one embodiment, the compounds and methods of the invention can be used to treat genitourinary tract infections (e.g., cystitis, uncomplicated UTI, acute uncomplicated pyelonephritis, complicated UTI, UTI in women, UTI in men, recurrent UTI, and asymptomatic bacteriuria).

In one embodiment, the invention provides for a method of treatment, either prophylactic or therapeutic treatment, of a subject or a patient population exposed to or at risk of exposure to an organism potentially important as an agent in bioterrorism by modulating the expression and/or activity of HIF—.

Exemplary therapeutic agents include, but are not limited to, small molecules, peptides, antibodies, ribozymes and antisense oligonucleotides.

In one aspect, the invention provides a method for preventing in a subject, a microbial infection by administering to the subject an agent which modulates the expression, concentration, and/or activity of HIF-1 or a combination of such agents. Subjects at risk for an infection can be identified, for example, based on the status of the subject (e.g., determining that a subject is immunocompromised) or based on the environmental conditions to which the subject is exposed, (e.g., determining that there is a possibility that the subject may be exposed to a certain agent). Administration of a prophylactic agent can occur prior to the manifestation of symptoms characteristic of an infection, such that an infection is prevented or, alternatively, delayed in its progression. The appropriate agent can be determined, e.g., based on screening assays described herein.

Another aspect of the invention pertains to methods for treating a subject suffering from an existing microbial infection. These methods involve administering to a subject an agent that modulates (e.g., inhibits) the expression and/or activity of HIF-1 or a combination of such agents. Many current infectious disease conditions are suboptimally treated or untreatable by conventional antibiotics for a variety of factors including compromised host immunity (e.g. neonates, pregnancy, the elderly, underlying illness such as diabetes or cancer, chemotherapy, AIDS, congenital immunodeficiencies), microbial resistance to conventional antibiotic agents (e.g. methicillin-resistant S. aureus, multidrug resistant Pseudomonas spp. or Mycobacterium tuberculosis, multidrug resistant fungi, vancomycin-resistant Enterococcus spp., infection of indwelling foreign bodies (e.g. catheters, prosthetic devices), or compromised epithelial integrity (e.g. burns, post-operative wounds). Modulation of HIF-1 activity to enhance the natural antimicrobial activity of host macrophages and neutrophils would offer a novel approach to treatment of these diverse patient groups.

In one embodiment, a second agent may be administered in conjunction with HIF-1 modulating agent of the invention. For example, the second agent can be one which is used clinically for treatment of the microbe. For example, in one embodiment, an antibiotic is co-administered with a HIF-1 modulating agent (e.g., is administered as part of the same treatment protocol) or is present on the same surface as the HIF-1 modulating agent.

In one embodiment, such a combination therapy is administered to prevent recurring infections (e.g., recurring urinary tract infections). In another embodiment, such a combination therapy is administered to reduce the amount of antibiotic or eliminate the need for one or more antibiotics for prophylaxis or treatment. In another embodiment, such a combination treatment prevents resistance to the antibiotic from developing in the microbe.

The compounds of the invention may be formulated in a composition suitable for use in environments including industry, pharmaceutics, household, and personal care. In an embodiment, the compounds of the invention are soluble in water. The modulating compounds may be applied or delivered with an acceptable carrier system. The composition may be applied or delivered with a suitable carrier system such that the active ingredient (e.g., HIF-1 modulating compound of the invention) may be dispersed or dissolved in a stable manner so that the active ingredient, when it is administered directly or indirectly, is present in a form in which it is available in a advantageous way.

Also, the separate components of the compositions of the invention may be preblended or each component may be added separately to the same environment according to a predetermined dosage for the purpose of achieving the desired concentration level of the treatment components and so long as the components eventually come into intimate admixture with each other. Further, the present invention may be administered or delivered on a continuous or intermittent basis.

A HIF-1 modulating compound when present in a composition will generally be present in an amount from about 0.000001% to about 100%, more preferably from about 0.001% to about 50%, and most preferably from about 0.01% to about 25%.

For compositions of the present invention comprising a carrier, the composition comprises, for example, from about 1% to about 99%, preferably from about 50% to about 99%, and most preferably from about 75% to about 99% by weight of at least one carrier.

The HIF-1 modulating compound of the invention may be formulated with any suitable carrier and prepared for delivery in forms, such as, solutions, (micro)emulsions, suspensions or aerosols. Generation of the aerosol or any other means of delivery of the present invention may be accomplished by any of the methods known in the art. For example, in the case of aerosol delivery, the compound is supplied in a finely divided form along with any suitable carrier with a propellant. Liquefied propellants are typically gases at ambient conditions and are condensed under pressure. The propellant may be any acceptable and known in the art including propane and butane, or other lower alkanes, such as those of up to 5 carbons. The composition is held within a container with an appropriate propellant and valve, and maintained at elevated pressure until released by action of the valve.

The compositions of the invention may be prepared in a conventional form suitable for, but not limited to topical or local application such as an ointment, paste, gel, spray and liquid, by including stabilizers, penetrants and the carrier or diluent with the compound according to a known technique in the art. These preparations may be prepared in a conventional form suitable for enteral, parenteral, topical or inhalational applications.

The HIF-1 modulating compound of the present invention may also be used in hygiene compositions for personal care. For instance, compounds of the invention can be used as an active ingredient in personal care products such as facial cleansers, astringents, body wash, shampoos, conditioners, cosmetics and other hygiene products. The hygiene composition may comprise any carrier or vehicle known in the art to obtain the desired form (such as solid, liquid, semisolid or aerosol) as long as the effects of the compound of the present invention are not impaired. Methods of preparation of hygiene compositions are not described herein in detail, but are known in the art. For its discussion of such methods, The CTFA Cosmetic Ingredient Handbook, Second Edition, 1992, and pages 5-484 of A Formulary of Cosmetic Preparations (Vol. 2, Chapters 7-16) are incorporated herein by reference. The hygiene composition for use in personal care comprise generally at least one modulating compound of the present application and at least one suitable carrier. For example, the composition may comprise from about 0.00001% to about 50%, preferably from about 0.0001% to about 25%, more preferably from about 0.0005% to about 10% by weight of the transcription factor modulating compound of the invention based on the weight percentage of the total composition.

In an alternate embodiment, the present invention is directed towards improving the efficacy of a variety of vaccines used in human or veterinary medicine through its action as an adjuvant. An adjuvant is defined as a chemical substance that is added to a vaccine formulation in order to enhance the immune response to vaccination. In current practice, there exist several types of adjuvants. Today the most common adjuvants for human use are aluminum hydroxide, aluminum phosphate and calcium phosphate. However, there are a number of other adjuvants based on oil emulsions, products from bacteria (their synthetic derivatives as well as liposomes) or gram-negative bacteria, endotoxins, cholesterol, fatty acids, aliphatic amines, paraffinic and vegetable oils. Recently, monophosphoryl lipid A, ISCOMs with Quil-A, and Syntex adjuvant formulations (SAFs) containing the threonyl derivative or muramyl dipeptide have been under consideration for use in human vaccines. Chemically, the adjuvants are a highly heterogeneous group of compounds with only one thing in common: their ability to enhance the immune response—their adjuvanticity. They are highly variable in terms of how they affect the immune system and how serious their adverse effects are due to the resultant hyperactivation of the immune system. Given our demonstration of the role of HIF-1 as a master regulator of innate immune function, coupled with the central role of macrophages in antigen presentation, agonists of HIF-1 added to killed, live-attenuated or recombinant vaccine formulations against bacterial, fungal or viral infection would serve to boost the efficacy of the vaccine in eliciting protective humoral (antibody-mediated) or cellular (T cell mediated) immunity. This method could also be applied to vaccines against other pathologies, diseases or disorders, including tumors, and the like.

### Sepsis

In an alternate embodiment, the present invention is directed towards treating a patient's suffering from disease related to sepsis. The present invention is involved in preventing, inhibiting, or relieving adverse effects attributed to sepsis over long periods of time and/or are such caused by the physiological responses to inappropriate sepsis present in a biological system over long periods of time. In addition, the present invention is related to identifying compounds for treating sepsis. In a preferred embodiment of the present invention, such compounds are HIF-1 antagonist, more preferably, a HIF-1α antagonist, which directly or indirectly inhibits the activity or level of HIF-1α. Those of skill in the art will readily recognize such compounds, including the anticancer HIF-1α drugs currently under development or to be developed.

Septic shock is a serious, abnormal condition that occurs when an overwhelming infection leads to low blood pressure and low blood flow. Vital organs, such as the brain, heart, kidneys, and liver may not function properly or may fail. Increased heart rate and decreased urine output from kidney failure may be one symptom. Septic shock occurs most often in the very old and the very young. It also occurs in people with underlying illnesses. Any bacterial organism can cause septic shock. Fungi and (rarely) viruses may also cause this condition. Toxins released by the bacteria or fungus may cause direct tissue damage, and may lead to low blood pressure and poor organ function. These toxins also produce a vigorous inflammatory response from the body which contributes to septic shock. Risk factors include: underlying illnesses, such as diabetes; hematologic cancers (lymphoma or leukemia) and other malignancies; and diseases of the genitourinary system, biliary system, or intestinal system. Other risk factors are recent infection, prolonged antibiotic therapy, and having had a recent surgical or medical procedure. Septic shock could be ameliorated by the administration of the anti-inflammatory and immunosuppressive agents of this invention in conceit with standard conventional antimicrobial therapy or the invention therapy.

### Inflammation

It was previously reported that HIF-1α regulates glycolysis in neutrophils and mononuclear phagocytes under both normoxic and hypoxic conditions. Utilizing HIF-1α-deficient mice, it was disclosed that HIF-1α regulates several key aspects of inflammation. It was subsequently suggested that HIF-1α inhibitors could be tested to determine if they could be employed to regulate inflammation. It has now been discovered that certain compounds can be utilized to treat inflammatory diseases, immune disorders and other such disorders. Accordingly, the invention also provides methods of preventing and/or treating a wide variety of inflammatory diseases, immune disorders, and skin aging. Certain of the diseases for which the methods are effective are discussed herein, as are factors and events which form a theoretical basis for the embodiments of the invention. However, this discussion is not in any way to be considered as binding or limiting on the present invention. Those of skill in the art will understand that the various embodiments of the invention may be practiced regardless of the model used to describe the theoretical underpinnings of the invention.

In an alternate embodiment, the present invention is directed towards treating a patient's suffering from disease related to pathological inflammation. The present invention is involved in preventing, inhibiting, or relieving adverse effects attributed to pathological inflammation over long periods of time and/or are such caused by the physiological responses to inappropriate inflammation present in a biological system over long periods of time. In a preferred embodiment of the present invention, there are provide methods for treating subjects suffering from inflammation, such method comprising administering a pharmaceutically effective amount of a HIF-1 antagonist, more preferably, a HIF-1α antagonist, which directly or indirectly inhibits the activity or level of HIF-1α. In addition, the present invention is related to identifying compounds for treating inflammation.

Such inflammation is characterized by a heightened response of inflammatory cells, including infiltrating leukocytes. Over time, such pathological inflammation often results in damage to tissue in the region of inappropriate inflammation.

The inflammation treated by the present methods, includes, but is not limited to, inflammation associated with an inflammatory disease, e.g., vascular inflammatory disorders, rheumatologic disorders, dermatologic inflammatory diseases, gastrointestinal inflammatory diseases and kidney disorders, including asthma, atherosclerosis, AIDS dementia, autoimmune diseases, diabetes, inflammatory bowel disease, transplant rejection, graft versus host disease, multiple sclerosis (especially to inhibit further demyelination), tumor metastasis, nephritis, atopic dermatitis, psoriasis, myocardial ischemia, chronic prostatitis, complications from sickle cell anemia, lupus erythematosus, and acute leukocyte mediated lung injury. Examples of the rheumatologic disorders include, but are not limited to, rheumatoid arthritis, osteoarthritis, vasculitis, sclereoderma, systemic lupus erythematosus and collagen vascular disorder. Other examples of diseases that can be treated by the present methods include, but are not limited to, restenosis, transplantation associated arteriopathy, Alzheimer's disease and fever, and aging and other cutaneous inflammatory reactions.

Arthritis is a collective term for inflammatory disease characterized by pain, swelling and stiffness in the joints and associated tissues. This is usually associated with immunological defects, inappropriate response to microbial antigens, or inflammatory changes provoked by chemical and mechanical damage. Over 200 types of arthritis have been described, of which rheumatoid arthritis, osteoarthritis, seronegative arthritis (e.g., ankylosing spondylitis, which involves the sacro-iliac joints), reactive arthritis and crystal arthritis are the most common. Descriptions of other forms of arthritis can be found in standard medical textbooks such as “Textbook of Medicine” (Eds Wyngaarden et al., 1992). Arthritis may be the dominant symptom of disease, as in osteoarthritis, or one symptom of complex autoimmune diseases such as lupus erythematosus or psoriasis.

Asthma is a chronic respiratory disease characterized by rapid onset of episodes of wheezing, coughing and airway obstruction that are sometimes relieved by bronchodilators or anti-inflammatory agents. It is believed that the inflammation following an initial allergic or toxic stimulus is a key feature of the disease. Eosinophil recruitment and activation is implicated as a central event. Currently, an estimated 25 million people in the US, Europe, and Japan suffer from asthma. It has become the most common chronic disease in industrialized countries and the prevalence, severity and mortality are rising. Allergic asthma is particularly prevalent in children where it may account for 90% of the disease.

Autoimmune diseases arise when the immune system reacts with endogenous proteins that are recognized as “foreign” antigens. This results in the formation of antibodies or immune T cells that can react with these antigens present in tissue to produce destructive changes. Immunosuppressive therapy has been shown to be effective in suppressing autoimmune reactions (Bach (1993) Trends Pharmacol. Sci. 14: 213-216). However, the efficacy of immunosuppressive therapy in the treatment of autoimmune diseases has been variable, and in general it has not been as effective as in organ transplantation or in treatment of specific immune disorders (e.g., the prevention of Rh hemolytic disease of the newborn).

One of the biological consequences of cutaneous inflammatory reactions in the skin is premature aging of the skin, manifested by clinical symptoms such as wrinkles, skin atrophy, abnormal pigmentations, and the like. The best defined cause of inflammatory reactions which leads to skin aging is exposure to UV radiation (UVR). Under acute conditions, sunburn is a clinical manifestation of over-exposure to UV light. Clinically, UVR induces skin redness, edema, and in more severe cases, pain and pruritis. The pathological changes in the skin due to UV exposure have been well-documented in the literature (see, Taylor and Sober (1996) “Sun Exposure and Skin Diseases”, Ann. Rev. Med. 47: 181-191.).

Similar to its response to UV irradiation, the epidermal keratinocyte can initiate and actively participate in the perpetuation of numerous cutaneous inflammatory reactions that follow exposure to a wide array of skin irritants (see, Kock A., et al., J. Exp. Med., 172(6):1609-14 (1990); Ansel J. et al., J. Invest. Dermatol., 94(6 Suppl):101S-107S (1990); Barker J N W N, et al., Lancet, 337(8735):2114 (1991); Nickoloff B. J., et al., J. Am. Acad. Dernatol., 30(4):535-46 (1994) and allergens (see, Piguet P. F., et al., J. Exp. Med, 173(3):673-9 (1991); Griffiths C. E. M. et al., Br. J. Dermatol, 124(6):519-26 (1991); Bromberg J. S., et al., J. Immunol., 148(11):3412-7 (1992); Enk A. H., et al., Proc. Natl. Acad. Sci. U.S.A., 89(4): 1398-402 (1992); Webb E. F., et al., J. Invest. Dermatol., 111(1):86-92 (1998)). Other cutaneous reactions include, eczema, allergic contact dermatitis (ACD), irritant contact dermatitis (ICD), and the like.

Additional diseases that can be treated by administration of the anti-inflammatory and immunosuppressive agents of the invention include, but are not limited to, respiratory diseases such as chronic obstructive airway disease and adult respiratory distress syndrome, neurological disorders such as multiple sclerosis and Alzheimer's disease, inflammatory bowel disease, Crohn's disease, ischemic/reperfusion injury, and Type I diabetes, graft-vs.-host disease, meningitis, gastritis and enteric infections, acne, and periodontal diseases. As the scientific knowledge of the pathophysiological factors responsible for various diseases progresses, it is anticipated that the utility of anti-inflammatory and immunomodulatory agents will expand beyond the current understanding.

The methods identified above can readily be employed in the present invention to identify compounds for preventing and/or treating a wide variety of inflammatory diseases, immune disorders, and skin aging. In addition, known HIF-1α inhibitors (see, e.g., Giaccia et al., Nat Rev Drug Discov. 2(10):803-11 (2003)) can be utilized as lead compounds or pharmaceutical compositions in the prevention and/or treatment of a wide variety of inflammatory diseases, immune disorders, and skin aging. Those of skill in the art will readily recognize methods and assays for identifying HIF-1α inhibitors of activity or levels, and the use thereof in the present invention.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The contents of all references, patent applications and patents, cited throughout this application are hereby expressly incorporated by reference. Each reference disclosed herein is incorporated by reference herein in its entirety. Any patent application to which this application claims priority is also incorporated by reference herein in its entirety.

The invention is further illustrated by the following examples of techniques and materials, which should not be construed as limiting the scope of the invention in any way.

### EXAMPLES

### Bacteria Induce HIF-1α Expression

Invasive pyogenic bacterial skin and soft tissue infections generate localized tissue ischemia, thrombosis, and necrosis and represent a formidable test of the adaptiveness of neutrophils and macrophages in hypoxic microenvironments. In this regard, a strain of the Gram-positive pathogen group A Streptococcus (GAS), isolated from a patient with necrotizing fasciitis (flesh-eating disease), was chosen as the primary model organism for most in vitro and in vivo challenges. We found that expression of HIF-1α was increased 4-fold in WT mouse macrophages following exposure to GAS under normoxic conditions (FIG. 1A). Indeed, GAS represented a more potent stimulus for HIF-1αinduction than hypoxia itself. The phenomenon of bacterial induction of HIF-1αunder normoxia was also observed with additional Gram-positive (methicillin-resistant Staphylococcus aureus, hereafter S. aureus) and Gram-negative (Pseudomonas aeruginosa, hereafter P. aeruginosa, and Salmonella typhimurium) bacterial species of medical importance (FIG. 1A).

We next evaluated whether the induction of HIF-1α protein by GAS corresponded to an increase in HIF-1α transcriptional gene activation. We measured HIF-1α-dependant transcription in macrophages derived from HRE-luciferase transgenic mice, which contain a luciferase reporter gene driven by 6 consecutive specific HRE sequences. As shown in FIG. 1B, a 3-fold increase in luciferase reporter activity was reached after incubating the macrophages for 18 hours in 1% oxygen or in the presence of known pharmacological inducers of HIF-1α, including desferrioxamine mesylate, cobalt chloride (CoCl₂), and L-mimosine (L-Mim). Incubation of the reported macrophages with live or heat-killed GAS bacteria at normoxia stimulated luciferase activity to levels comparable to or greater than those of hypoxia (FIG. 1B).

### HIF-1α Regulates Microbicidal Capacity of Myeloid Cells.

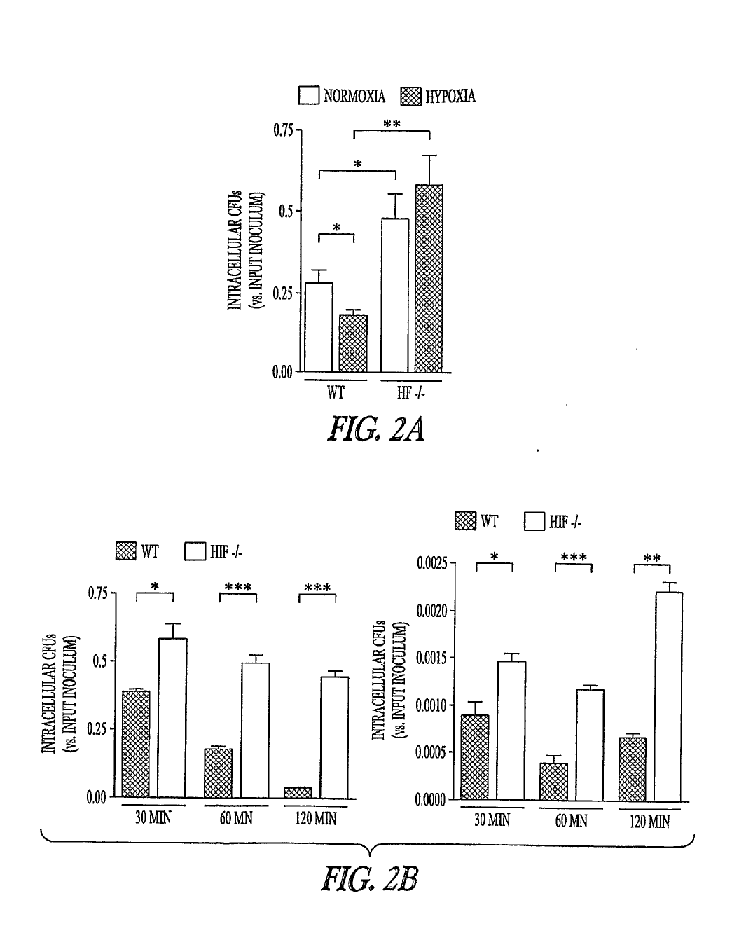

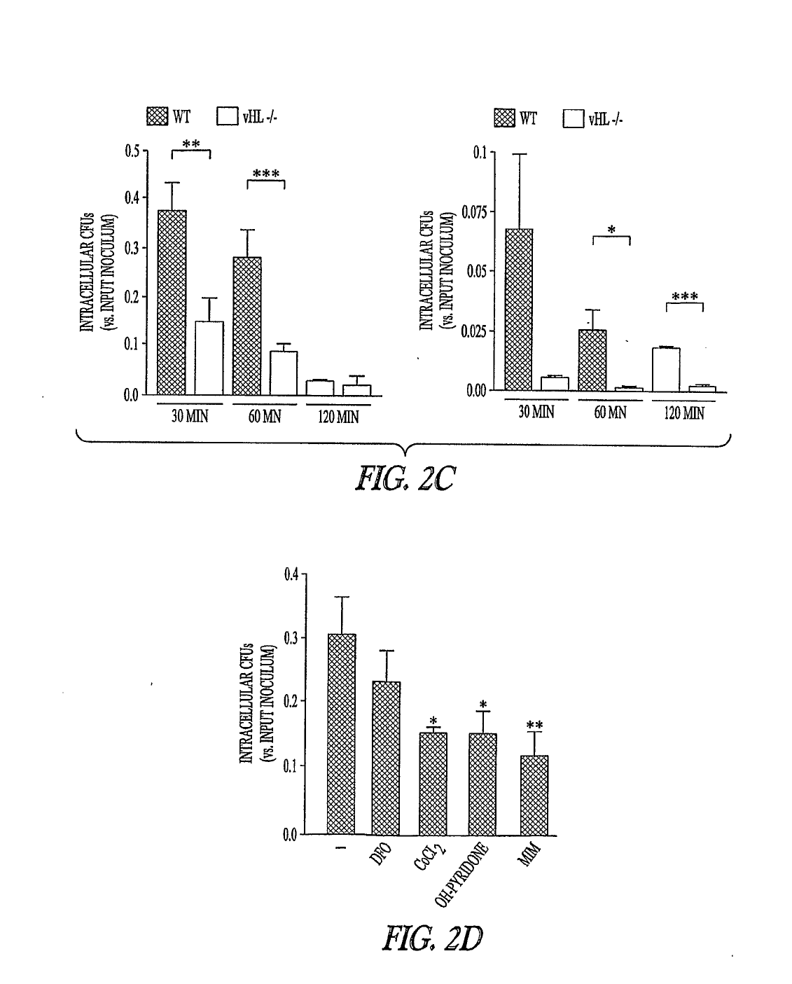

To assess the functional consequences of HIF-1α activation, we used an antibiotic protection assay to calculate intracellular killing of GAS by WT macrophages compared with killing by those derived from the bone marrow of HIF-1α-lysMcre mice (11). Here, targeted deletion of the HIF-1α gene has been created via crosses into a background of cre expression driven by the lysozyme M promoter (lysMcre), allowing specific deletion of the transcription factor in the myeloid lineage (11). As shown in FIG. 2A, intracellular killing of GAS by WT macrophages was increased under hypoxia, providing initial indication that HIF-1α may be involved in the bactericidal process. This result was especially notable because the facultative GAS bacteria lack oxidative phosphorylation and grow more rapidly under anaerobiasis (12). We found that, compared to WT cells, macrophages from HIF-1α-null mice showed a 2-fold decrease in GAS intracellular killing under normoxia and a 3-fold decrease in GAS intracellular killing under hypoxia (FIG. 2A). Time-course studies showed that the killing defect observed in HIF-1α-null macrophages increased over time, such that 15-fold more viable bacteria were present within HIF-1α deleted cells by the last time point of 120 minutes (FIG. 2B). Macrophage killing of the Gram-negative bacterium P. aeruginosa was likewise impaired upon deletion of HIF-1α (FIG. 2B).

As a complementary analysis of the linkage of myeloid cell bactericidal functions with HIF-1α transcriptional control, we explored the effects of increased HIF-1α activity on bacterial killing by using macrophages derived from vHL-null mice. vHL is a key regulator of HIF-1α turnover; these mice have constitutively high levels of HIF-1 activity in the deleted cell population (11). We found that vHL-null macrophages showed increased intracellular killing of GAS and P. aeruginosa compared with WT cells across multiple time points (FIG. 2C). Similar differences were observed in macrophage bactericidal assays that omitted antibiotics and instead employed vigorous washing to quantify total surviving cell-associated GAS or P. aeruginosa. Macrophage populations isolated from WT, HIF-1α-null, and vHL-null mice both included more than 99.5% differentiated macrophages by flow cytometric analysis, and Trypan blue straining showed similar levels of macrophage viability (98-99%) throughout the GAS-killing assays. These controls suggest that there exists an intrinsic defect in the bactericidal activity of HIF-1α-null cells that cannot be attributed to differences in the purity or viability of the explanted cell populations.

Finally, we treated WT macrophages with a number of known pharmacologic inducers of HIF-1α that each act directly or indirectly to inhibit prolyl hydroxylase targeting of HIF-1α for ubiquitination. These included the iron chelator desferrioxamine, CoCl₂, L-Mim, and 3-hydroxy-1,2-dimethyl-4(1H)-pyridone (OH-pyridone) (13). The addition of each of these agents increased intracellular killing of GAS by WT macrophages (FIG. 2D). Assays were performed using a concentration of each agonist and exposure time that did not affect bacterial viability.

### Myeloid Cell HIF-1α Production is Important for Control of GAS Infection In Vivo.

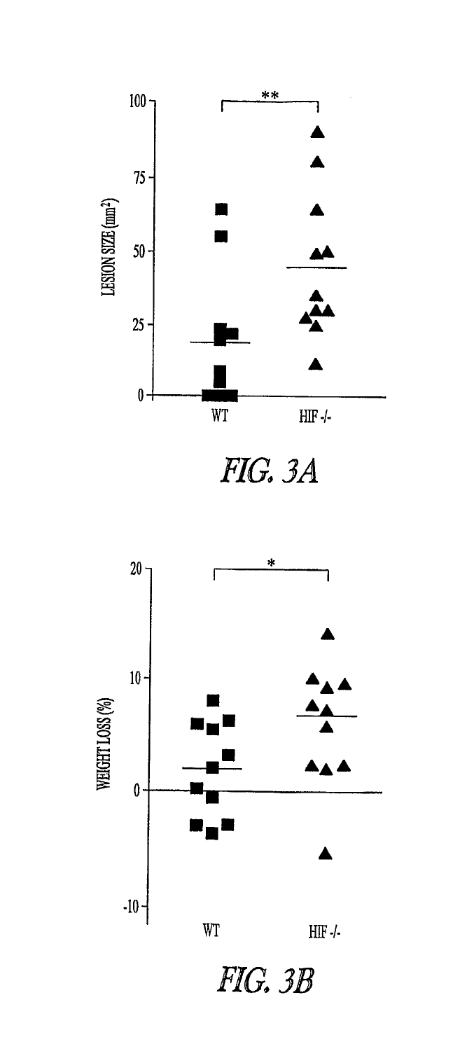

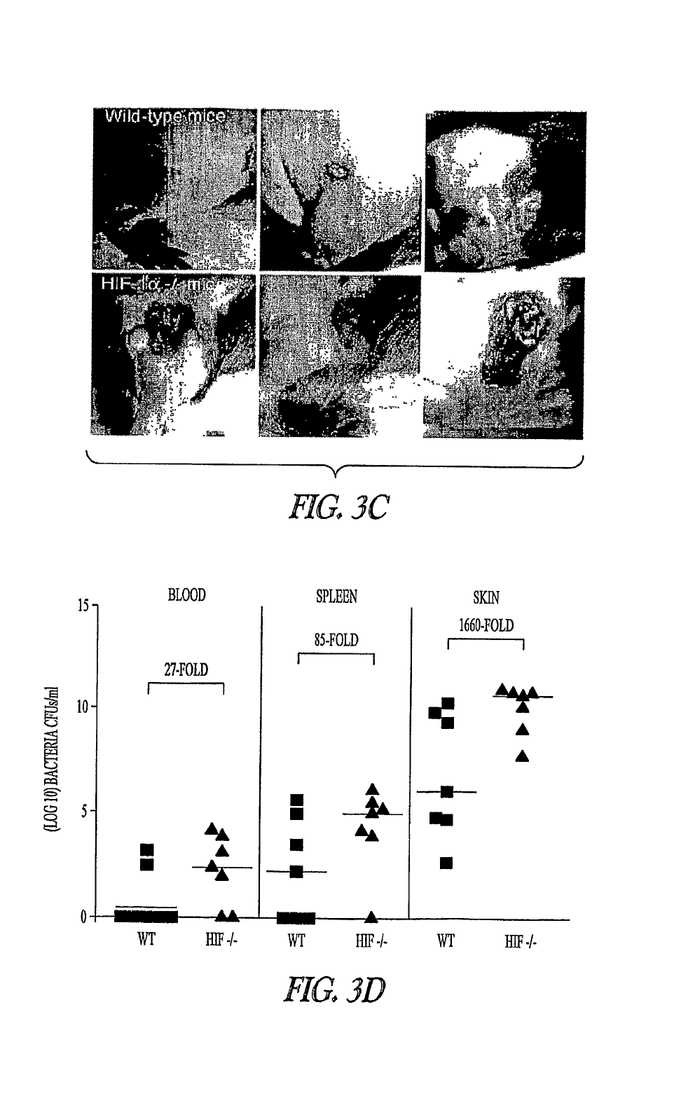

We chose an animal infection model of GAS-induced necrotizing soft tissue infection for directly testing myeloid cell microbicidal function in vivo. We introduced the GAS inoculum subcutaneously into a shaved area on the flank of WT and HIF-1α−/− male littermates and followed progression of the infection over 96 hours. We found that mice with a tissue-specific deletion of HIF-1α in macrophage and neutrophils developed significantly larger necrotic skin lesions and experienced greater weight loss than WT mice (FIG. 3, A and B). Representative gross appearance of the necrotic lesions in WT and HIF-1α myeloid-null mice is shown in FIG. 3C. We next asked whether myeloid cell production of HIF-1α was important in limiting the ability of GAS to replicate within the necrotic skin tissues and to disseminate from the initial focus of infection into the bloodstream and systemic organs. Mice were sacrificed at 96 hours after inoculation and quantitative bacterial cultures performed on the skin ulcer (or site of inoculation if no ulcer developed), blood, and spleen (FIG. 3D). Approximately 1,660-fold greater quantities of GAS were present in the skin biopsies of HIF-1α-null mice compared with those of WT mice. Similarly, 27-fold (blood) or 85-fold (spleen) more bacteria were isolated in systemic cultures from HIF-1α-null mice compared with WT mice. Our findings indicate that the presence of HIF-1α transcriptional control in neutrophils and macrophages is important in limiting the extent of necrotic tissue damage and preventing systemic spread of microbial infection.

### HIF-1α is Not Critical for Neutrophil Endothelial Transcytosis or Oxidative Burst Function.

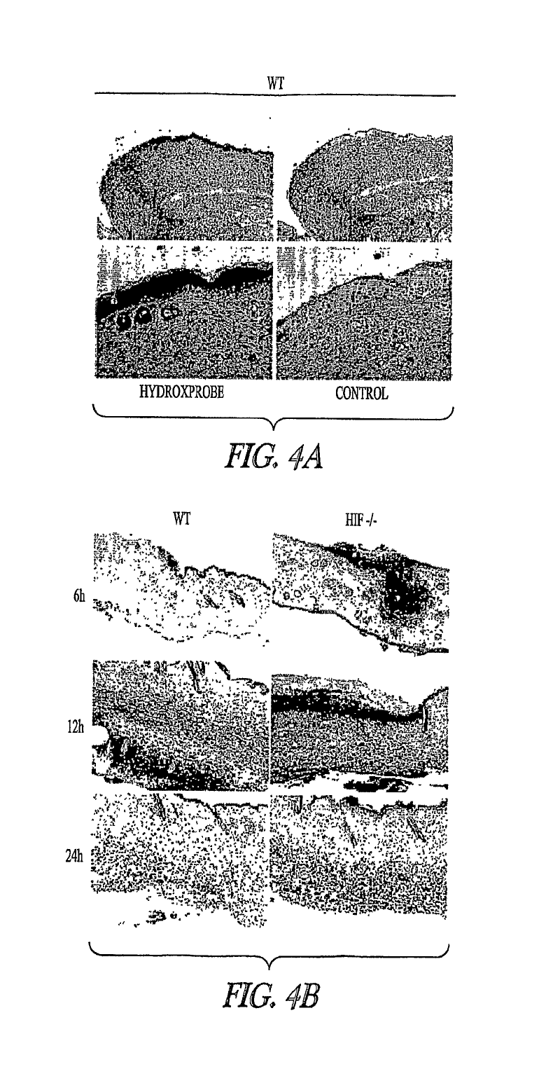

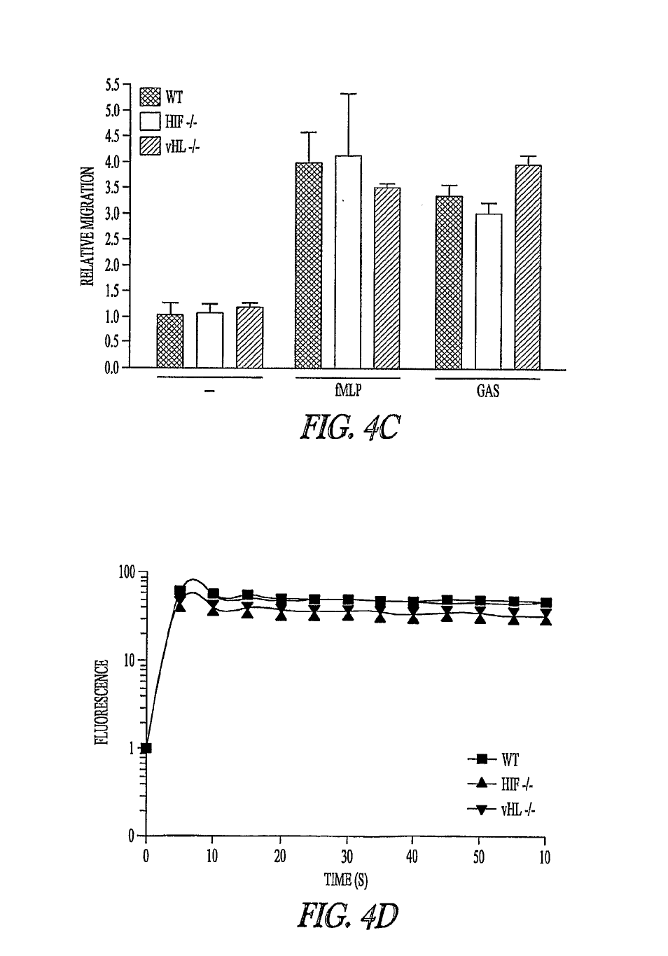

We next began a series of experiments to probe the potential cellular and molecular mechanisms through which HIF-1α may support myeloid cell functional killing capacity in vitro and in vivo. Although histopathologic examination of the biopsies from the necrotic ulcers generated by GAS revealed clear tissue ischemia by HypoxyProbe (FIG. 4A), the observed immune defect of HIF-1α-null animals did not appear to reflect impaired phagocyte recruitment, since similar numbers of neutrophils were observed on immunostaining of the skin tissue of WT compared with that of HIF-1α-null mice at 6, 12, and 24 hours after infection (FIG. 4B). The latter finding differed qualitatively from our previous study, in which decreased neutrophil infiltration was seen in skin tissue of HIF-1α after chemical irritation with the phorbol ester tetradecanoyl phorbol acetate (11), and from the prediction that might be derived from HIF-1α control of 132 integrin expression (14). We speculate that the stimulus to neutrophil migration elicited by microbial infection is perhaps stronger and more complex (i.e., involving more pathways) than that of chemical irritation such that the any contribution of HIF-1α may be muted in comparison to its effects on microbial killing. To explore further whether the migratory capacity of WT and HIF-1α-null neutrophils toward a bacterial stimulus was indeed unaffected, we measured the rate of transcytosis across murine endothelial cell monolayers following stimulation by GAS or the bacteria-derived chemotactic peptide N-formyl-methionyl-leucyl-phenylalanine (fMLP). In these assays, we also found no significant difference in transendothelial migration between the activated WT, HIF-1α-null, and vHL-null murine neutrophils (FIG. 4C).

The production of reactive oxygen metabolites generated by lysosomal NADPH oxidases in a process known as the respiratory burst is a major mechanism of bacterial killing. However, circulating neutrophils derived from HIF-1α-deficient or vHL-deficient mice were similar to WT neutrophils in oxidative burst activity (FIG. 4D). Thus, the defect in innate immunity to GAS infection observed in HIF-1α myeloid-null mice could not be attributed to impairment in oxidative burst function.

### Production of Granule Proteases and Antimicrobial Peptides is Regulated by HIF-1α

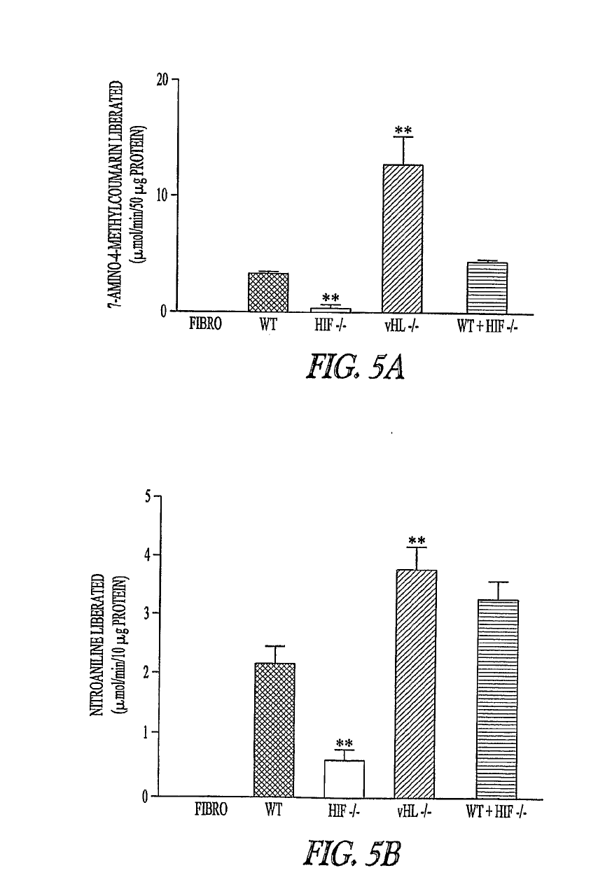

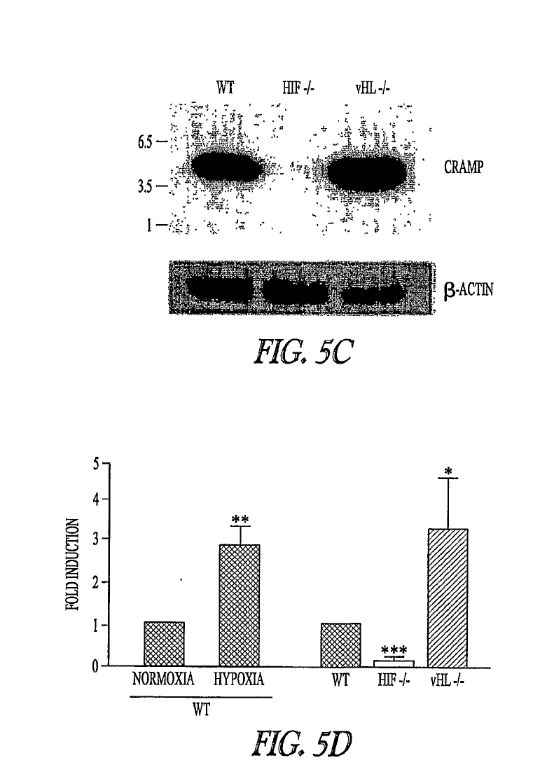

Granule proteases are increasingly recognized as an important component of myeloid cell antimicrobial activity. Neutrophil elastase (NE) and cathepsin G are abundant serine proteases concentrated in the granules that are primarily destined to fuse to phagosomes and form phagolysosomes. Gene targeting of elastase in mice has directly supported a role of NE in host innate immune defense (15), and accumulating evidence suggests a similar role for cathepsin G (16-18). Patients with Chediak-Higashi syndrome lack NE and suffer recurrent bacterial infections. To determine whether HIF-1α has an impact on neutrophil production of granule proteases, we measured NE and cathepsin G activity in WT, HIF-1α-null, and vHL-null blood neutrophils. Protease activity was measured using a synthetic peptide substrate containing recognition sites for each molecule to allow either fluorometric (NE) (FIG. 5A) or spectrophotometric (cathepsin G) detection (19) (FIG. 5B). HIF-1α-null neutrophils showed decreased enzymatic activity of each granule protease compared with WT neutrophils while vHL-null neutrophils exhibited increased protease activity. Mixing experiments with WT and HIF-1α-null macrophages excluded the possibility that HIF-1α-null neutrophils produce a greater amount of an (unknown) inhibitor rather than less of the granule proteases (FIG. 5, A and B).

The production of proteases by neutrophils may exert direct antimicrobial effects or, alternatively, may serve to activate cationic antimicrobial peptides from their inactive precursor forms (20, 21). An important component of innate immune defense in mammals is the cathelicidin family of antimicrobial peptides (22). These gene-encoded “natural antibiotics” exhibit broad-spectrum antimicrobial activity and are produced by several mammalian species on epithelial surfaces and within the granules of phagocytic cells. Proteolytic cleavage of an inactive precursor form to release the mature C terminal antimicrobial peptide is accomplished by proteases, such as elastase, upon degranulation of activated neutrophils (23). Mice have a single cathelicidin, cathelicidin-related antimicrobial peptide (CRAMP), which closely resembles the single human cathelicidin (LL-37). Importantly, we demonstrated in earlier experiments using the murine model of necrotizing skin infection that endogenous production of CRAMP was essential for mammalian innate immunity to GAS (24). We performed experiments to identify whether production or activation of CRAMP was under HIF-1α control. Lysates from WT, HIF-1α, HIF-1α-null, and vHL-null peritoneal neutrophils were analyzed by SDS-PAGE and immunoblotted with a rabbit anti-mouse CRAMP antibody against the CRAMP mature peptide. HIF-1α deletion led to a dramatic reduction of the active mature form of cathelicidin compared with WT neutrophils while CRAMP was expressed at higher levels in vHL-deficient neutrophils (FIG. 5C). Regulation of cathelicidin expression occurred at least in part at the mRNA level, as CRAMP transcript levels are reduced by 80% in HIF-1α-null macrophages, and conversely increased with loss of vHL (FIG. 5D). As would be expected, CRAMP mRNA was also increased by exposure of the neutrophils to hypoxia (FIG. 5D). Thus, the production and activation of cathelicidin antimicrobial peptides represents an additional myeloid cell killing mechanism that is affected by alterations in the HIF-1α pathway.

### HIF-1α is a Principal Regulator of NO Production in Response to Microbial Infection.

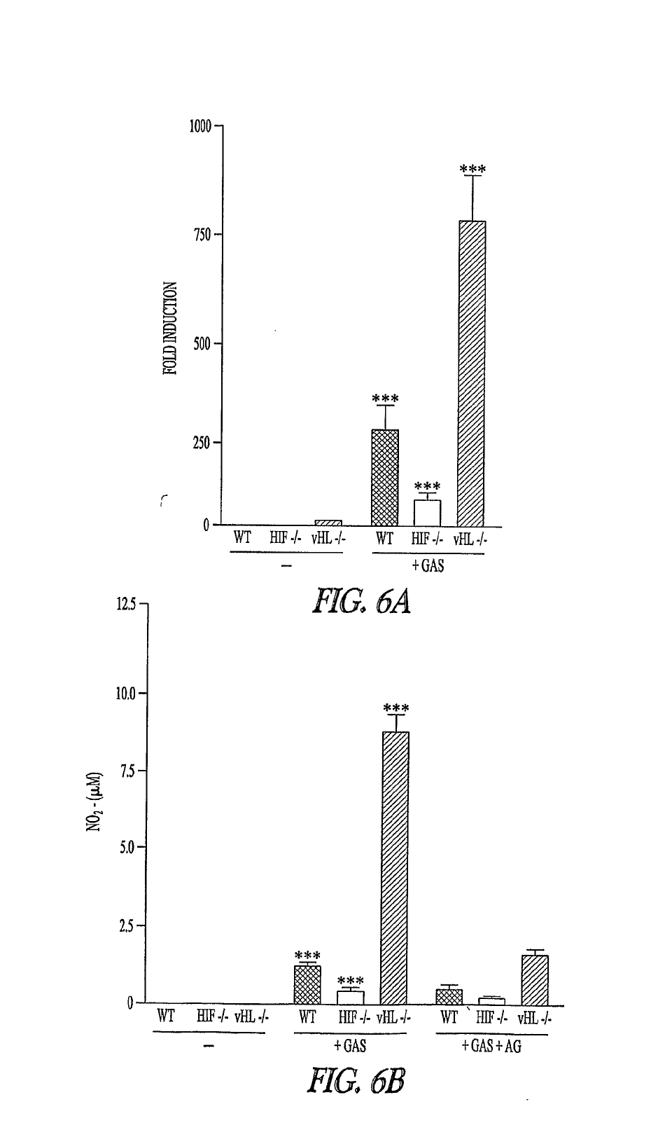

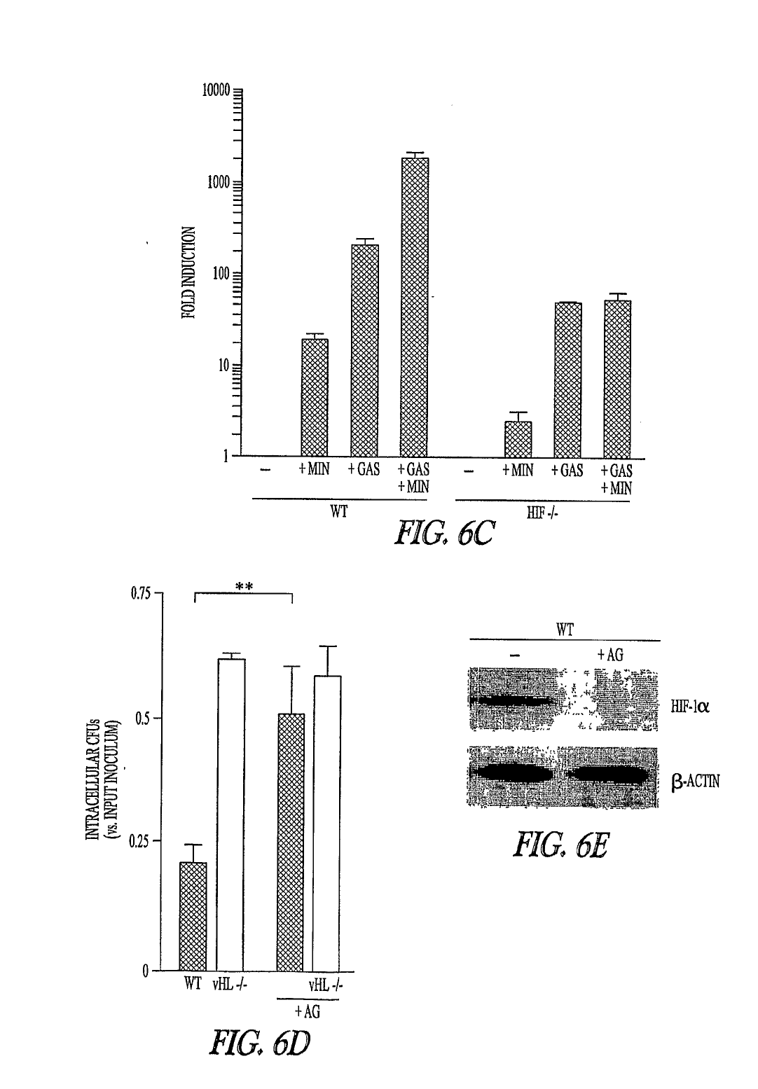

NO is known to exert antimicrobial properties against a variety of microbial species (25). Nitric oxide is enzymatically produced by NOS through the oxidation of arginine, and mice deficient in iNOS are more susceptible to microbial infection (26, 27). It has been well documented that HIF-1α is a transcriptional activator of iNOS expression (28-30), but no studies have examined this linkage in the context of microbial infection. Here we found that exposure of macrophages to GAS increased iNOS mRNA production approximately 250-fold (FIG. 6A). Deletion of HIF-1α resulted in an approximately 70% reduction in iNOS gene transcription, while deletion of vHL led to a marked increase in iNOS mRNA levels (FIG. 6A). Measurement of nitrite in cell culture supernatants confinned that the observed differences in iNOS induction translated directly to differences in NO production (FIG. 6B). Addition of the NOS inhibitor 1-amino-2-hydroxyguanidine, p-toluenesulfate (AG) significantly inhibited the production of NO in response to GAS (FIG. 6B). WT macrophages treated with L-Mim, a pharmological inducer of HIF-1α showed greatly increased expression of iNOS mRNA, but this increased expression was very low in HIF-1α-null cells (FIG. 6C), confirming a dependency of the observed effect on the presence of the transcription factor. These experiments indicate that augmentation of iNOS expression and subsequent microbicidal (microbial killing) (FIG. 2C) can be pharmacologically induced through increased HIF-1α expression.

To establish the functional importance of HIF-1α-induced iNOS expression and NO production, we performed macrophage microbicidal assays in the presence or absence of AG. FIG. 6D shows that AG inhibited the bactericidal activity of WT macrophages but did not further suppress the poor bactericidal activity of HIF-1α-null macrophages. We found similar results using the iNOS-specific inhibitor 1400 W. It has recently been demonstrated that NO, as well as certain reactive oxygen species, cytokines, and growth factors, can participate in stability regulation of HIF-1α and HIF-1 transactivation during normoxia (31-36). As seen in FIG. 6E, we found that inhibition of iNOS by AG blocked the ability of GAS exposure to generate increased levels of HIF-1α in WT macrophages. Thus, HIF-1α induces the production of NO, which not only acts as a key element in microbial killing, but also serves as a regulatory molecule that further stabilizes HIF-1α. This places HIF-1α at the center of an amplification loop during the innate immune response of myeloid cells to microbial infection.

HIF-1α Regulates Myeloid Cell TNF-α Production through a NO-Dependent Process.

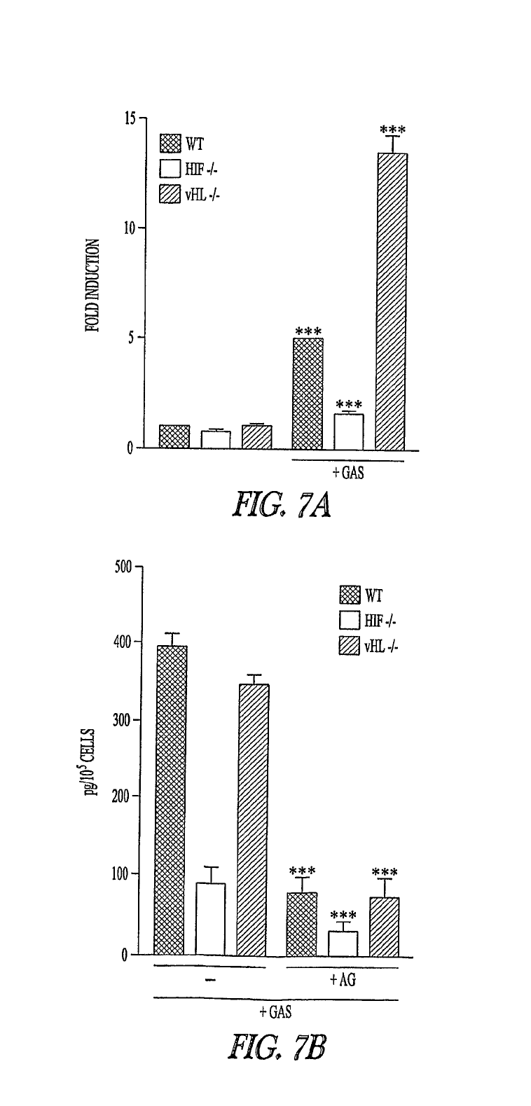

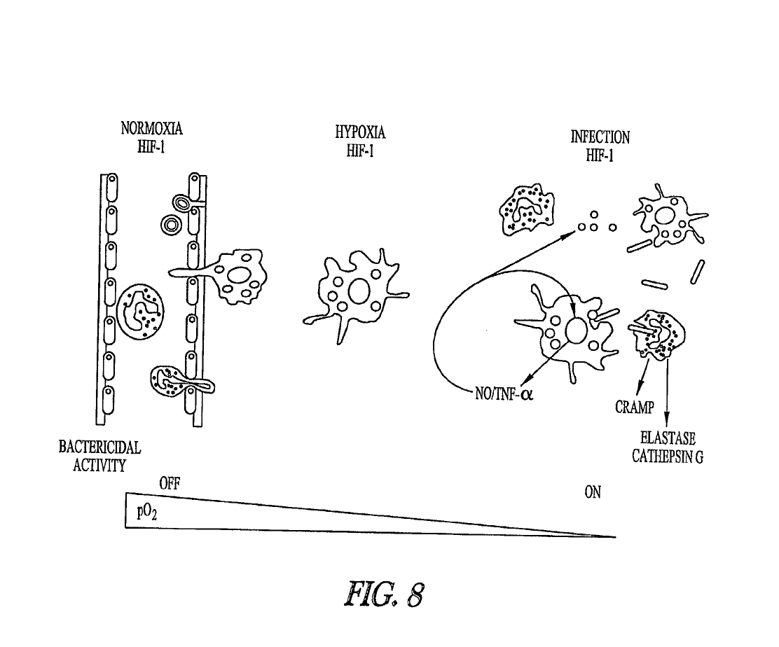

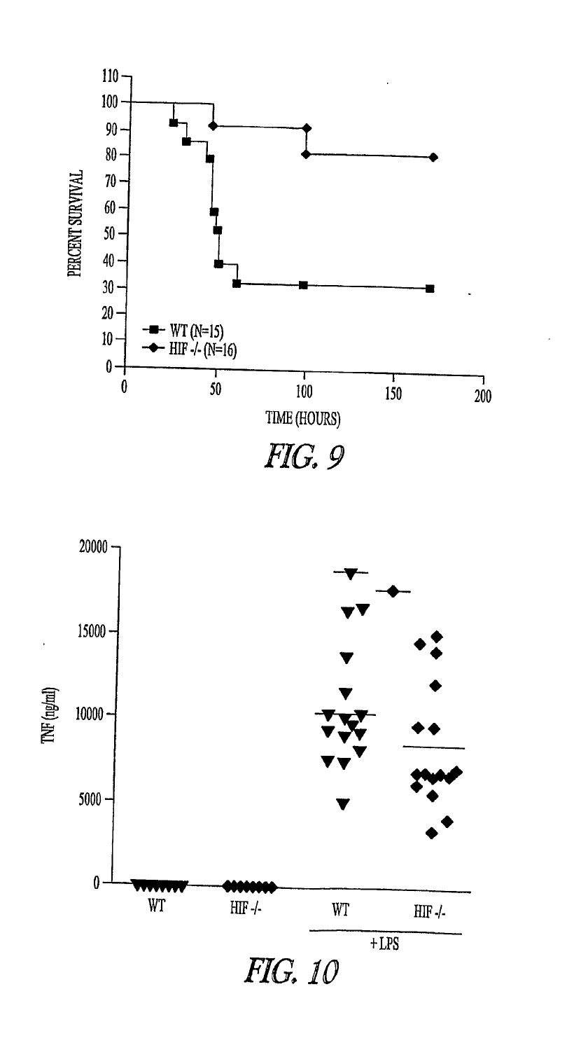

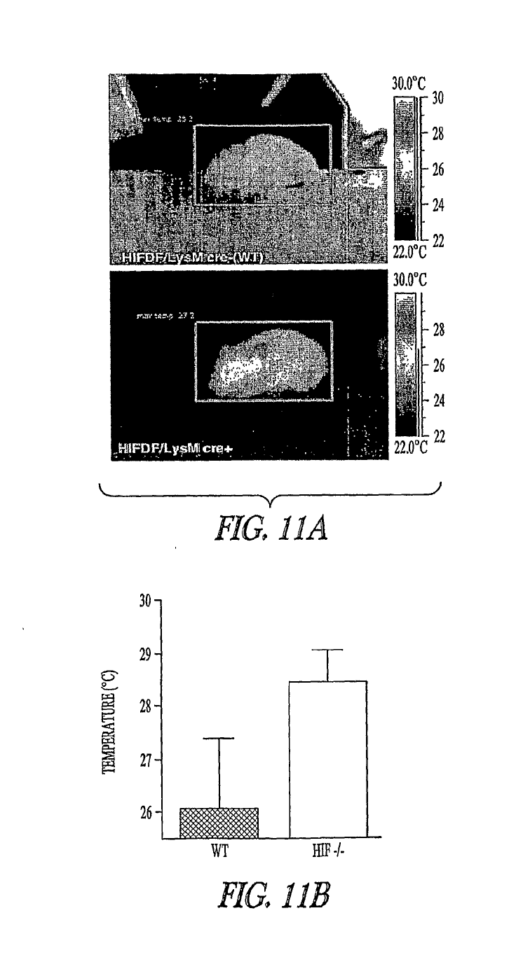

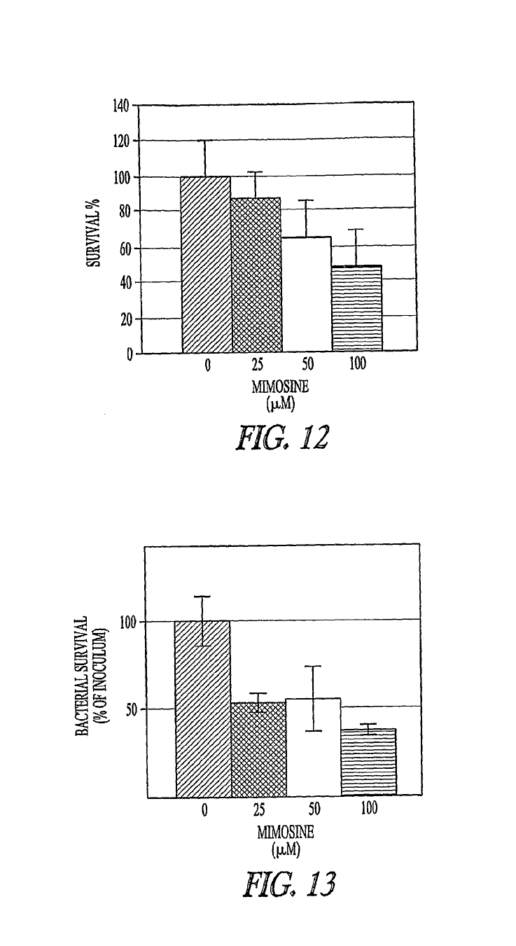

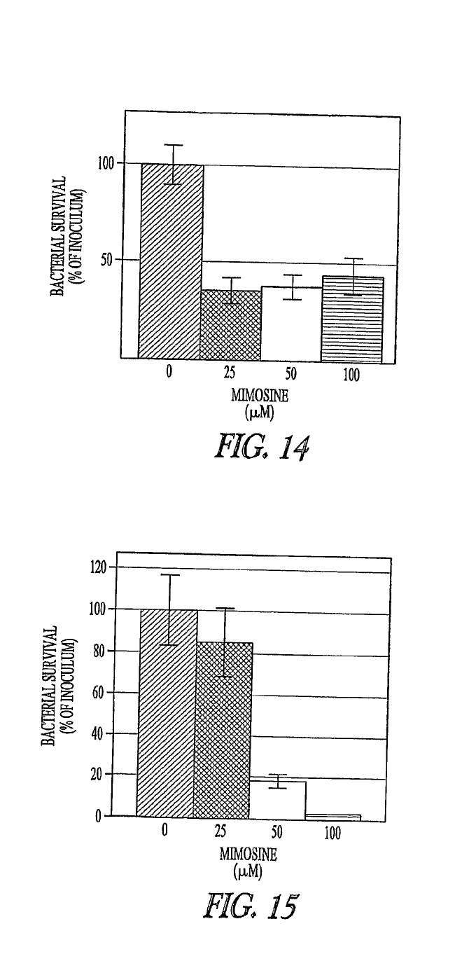

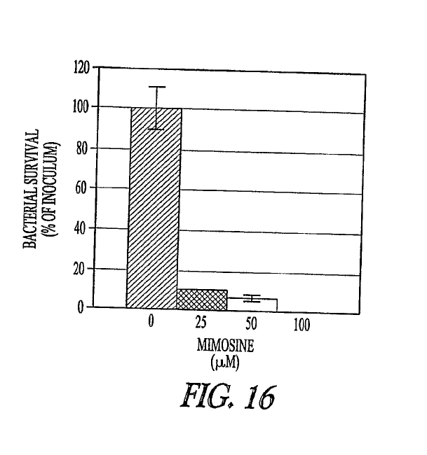

We next examined the expression pattern of TNF-α, a cytokine involved in the augmentation of inflammatory responses to microbial infection. Indeed, development of GAS-necrotizing fasciitis has been reported as a complication of anti-TNF-α therapy (37). As shown in FIG. 7A, GAS strongly induced TNF-α mRNA production in WT macrophages. This transcriptional response was severely diminished in HIF-1α-null macrophages and upregulated in vHL-null macrophages. Whereas basal levels of TNF-α protein expression were similar in WT, HIF-1α, and vHL-null macrophages, loss of HIF-1α also strongly depressed the rapid secretion of TNF-α protein in response to GAS (FIG. 7B). As NO is markedly induced under GAS stimulation, TNF-α induction by GAS may rely on HIF-1-dependent NO production. ELISA for secreted TNF-α demonstrated reduced amounts of TNF-α protein in conditioned supernatants of WT, HIF-1α, and vHL-deficient macrophages in the presence of the iNOS inhibitor AG (FIG. 7B). This finding indicates that NO production, acting in a HIF-1α controlled manner, contributes significantly to the macrophage TNF-α response to microbial infection.

### Harvest of Neutrophils, Macrophages, and Blood Leukocytes.

Neutrophils were either isolated from the peritoneal cavity 3 hours after injection of thioglycollate as previously described (11, 50) or derived from bone marrow as described (51). To isolate BM-derived macrophages, the marrow of femurs and tibias of WT, HIF-1 myeloid-null, or vHL myeloid-null mice were collected. Cells were plated in DMEM supplemented with 10% heat-inactivated FBS and 30% conditioned medium (a 7-day supernatant of fibroblasts from cell line L-929 stably transfected with an M-CSF expression vector). Mature adherent BM cells were harvested by gentle scraping after 7 days in culture. To isolate blood leukocytes, 200-500 μl of whole blood was collected by retroorbital bleed into cold EDTA-coated capillary tubes (Terumo Medical Corp.). Cells were centrifuged, erythrocytes were lysed using ACK RBS lysis buffer (0.15 M NH4Cl, 10.0 mM KHCO3, 0.1 mM EDTA), and unlysed cells were washed once with 1 ml PBS1% BSA.

### Bacterial Strains and Media.

GAS strain 5448 is an Ml serotype isolate from a patient with necrotizing fasciitis and toxic shock syndrome (52). Additional bacterial strains were obtained from the ATCC Bacteriology collection, specifically methicillin-resistant S. aureus (ATCC 33591, designation 328), S. typhimurium (ATCC 1311), and P. aeruginosa (ATCC 27853, designation Boston 41501). GAS was propagated in Todd-Hewitt broth (THB) (Difco; BD Diagnostics) and other strains in Luria-Bertani broth.

Bacterial killing assays. GAS were grown to logarithmic phase in THB to OD600=1×108 cfu/ml. Bacteria were added to macrophages at an MOI of 2.5 bacteria/cell and intracellular killing assessed using an antibiotic protection assay (11, 53) or, alternatively, total cell-associated bacteria measured by vigorous washing with PBS×3 to remove nonadherent bacteria. At the end of the assay, total cell lysate was plated on THB agar for enumeration of CFU. Comparable studies were performed with P. aeruginosa at an MOI of 25. To assess macrophage viability, the monolayers were washed with PBS and incubated with 0.04% Trypan blue for 10 minutes at 37° C. As specified in the FIG. 2 legend and in Results, macrophages were preincubated with L-Mim (800 μM), OH-pyridone (150 μM), desferrioxamine mesylate (100 μM), or CoCl₂ (100 μM) for 5 hours prior to the killing assay; each drug level was known to be sufficient for HIF-1 induction (13). Absence of bacterial inhibition was tested by incubating the drugs at the above concentrations with GAS (˜105) at 37° C. for 1-24 hours. Mouse model of GAS infection.

An established model of GAS subcutaneous infection was adapted for our studies (24, 54). Briefly, 100 μl of a midlogarithmic growth phase (˜107 cfu) of GAS was mixed with an equal volume of sterile Cytodex beads (Sigma-Aldrich) and injected subcutaneously into a shaved area on the flank of 5- to 8-week-old male littermates. Mice were weighed daily and monitored for development of necrotic skin lesions. After 96 hours, skin lesions, spleen, and blood (via retroorbital bleeding) were collected and homogenized in 1:1 mg/ml PBS. Serial dilutions of the mixture were plated on THB agar plates for enumeration of CFUs.

### Immunohistochemistry.

Lesions were processed, embedded into paraffin, and routine sections (5 μm) cut. Immunohistochemistry was performed with an antibody specific for neutrophils (purified anti-mouse neutrophils mAb; Accurate Chemical & Scientific Corp.) as described (55). To assess development of hypoxic regions within the lesions, mice were injected intraperitoneally with 60 mg/kg (weight/volume in PBS) pimonidazole (Hydroxyprobe-1, Natural Pharmacia International Inc.) 2 hours prior to sacrifice. Immunohistochemistry was performed with Hydroyprobe-1 mouse monoclonal antibody as reported (56). Reverse transcription and real-time quantitative PCR.

First-strand synthesis was obtained from 1 μg of total RNA isolated with Trizol reagent (Molecular Research Center Inc.) by the SuperScript system (Invitrogen Corp.), employing random primers. For real-time PCR (RT-PCR) analyses, cDNAs were diluted to a final concentration of 10 ng/μl and amplified in a TaqMan Universal Master Mix, SYBR Green (Applied Biosystems). cDNA (50 ng) was used as a template to determine the relative amount of mRNA by RT-PCR in triplicate (ABI PRISM 7700 Sequence Detection System; Applied Biosystems), using specific primers with the following sequences: iNOS forward 5′-ACCCTAAGAGTCACAAAATGGC-3′; iNOS reverse 5′-TTGATCCTCACATACTGTGGACG-3′; TNF-forward 5′-CCATTCCTGAGTTCTGCAAAGG-3′; TNF-reverse 5′-AGGTAGGAAGGCCTGAGATCTTATC-3′; TNF-probe 5′-6[FAM]AGTGGTCAGGTTGCCTCTGTCTCAGAATGA[BHQ]-3′; CRAMP forward 5′-CTTCAACCAGCAGTCCCTAGACA-3′; CRAMP reverse 5′-TCCAGGTCCAGGAGACGGTA-3′; elastase forward 5′-TGGCACCATTCTCCCGAG-3′; elastase reverse 5′-CATAGTCCACAACCAGCAGGC-3′; β-actin forward 5′-AGGCCCAGAGCAAGAGAGG-3′; and β-actin reverse 5′-TACATGGCTGGGGTGTTGAA-3′.

### Nitrite Determination.

The concentration of nitrite (NO2-), the stable oxidized derivative of NO, was determined in 100-μl aliquots of cell culture supernatants transferred to 96-well plates. Essentially, 100 μl of Griess reagent (1% sulfanilamide, 0.1% naphthylene diamine dihydrochloride, 2% H3PO4) was added per well, and the absorbances were measured at 540 nm in a microplate reader. Sodium nitrite diluted in culture medium was used as standard.

### Elastase and Cathepsin G Assays.

For elastase measurement, 100 μl of 0.2M Tris-HCL (pH 8.5) containing 1M NaCl was mixed with 50 μg of blood leukocytes lysed in HTAB buffer containing 0.1M Tris-Cl, pH 7.6, 0.15 M NaCl, and 0.5% hexadecyltrimethylammonium bromide. Next, 100 μl of MeOSuc-Ala-Ala-Pro-VaiNmec dissolved in DMSO at 20 mM was added to the buffered enzyme to start the reaction. The hydrolysis of the substrate was monitoring spectrofluorometrically using excitation at 370 nm and emission at 460 nm. For cathepsin G quantitation, 20 μl of Suc-Ala-Ala-Pro-Phe-NphNO2 dissolved at 20 mM in DMSO was diluted to 180 μl with 0.1 M HEPES buffer, pH 7.5. The reaction was started by the introduction of 10 μg of blood neutrophils lysed in HTAB buffer, and the increase in A410 was monitored.

### Western Blot Studies.

Peritoneal neutrophils or bone marrow-derived macrophages inoculated with GAS were harvested and washed with PBS, and proteins were extracted with HTAB or RIPA buffers. Protein concentration was calculated using the Bio-Rad assay (Bio-Rad Laboratories). Fifty milligrams of protein or nuclear extracts were loaded on a 10% Tris-tricine gel in an MES buffer (Invitrogen Corp.) or 3-8% Tris-tricine gel in a Tris-acetate buffer (Invitrogen Corp.) for CRAMP and HIF-1 Western blot respectively. Proteins were transferred to a nitrocellulose membrane; the membrane was blocked in 5% nonfat milk in 0.2% Tween TBS and then incubated in primary Ab diluted in 5% nonfat milk. The primary Abs used were rabbit anti-mouse CRAMP against the CRAMP mature peptide and rabbit anti-mouse HIF-1 (Cayman Chemical Co.). The secondary Ab was peroxidase-conjugated goat anti-rabbit (DAKO Corp.). Immunoreactive proteins were detected using the ECL chemiluminescent system (Amersham Biosciences).

### Reporter Assay.

Macrophages were derived from the marrow of femurs and tibiae of transgenic HRE-luciferase mice as described above. The luciferase reporter gene in these mice is driven by 6 specific HRE sequences. Cells were incubated with GAS or heat-inactivated GAS for 18 hours. As a positive control, macrophages were incubated under hypoxia (1%) or with the addition of L-Mim (800 μM), desferrioxamine mesylate (150 μM), or CoCl₂ (150 μM) during the same period of time. Cells were then washed out with PBS, and luciferase assay was performed by using the Bright-Glo Luciferase Assay kit (Promega Corp.). Luciferase activities were measured using a luminometer.

### Oxidative Burst Assay.

Isolated total blood leukocytes were resuspended at 4° C. in approximately 200 μl endotoxin- and pyrogen-free PBS, lacking Ca2+ and Mg2+ but containing 5 mM glucose. Immediately before the oxidative burst assay, 200 μL of PBS at 37° C. containing 1.5 mM Mg2+ and 1.0 mM Ca2+ were added to the cell suspension. Oxidative burst activity was measured by using the Fc OxyBURST Green assay reagent (Invitrogen Corp.) according to the manufacturer's instructions.

### Endothelial Cell Transmigration Assay.

Thioglycolate-stimulated neutrophils were added to the upper chamber of a Transwell membrane (Corning HTS) coated with a primary murine pulmonary endothelial monolayer. The chemokine fMLP (8 ng/μl) or GAS (MOI=10:1) was added to the lower well. The number of neutrophils migrating to the lower chamber was counted after 1 hour of incubation at 37° C.

### Reagents.

AG and 1400 W were purchased from EMD Biosciences. L-Mim, OH-pyridone, desferrioxamine mesylate, and CoCl₂ were purchased from Sigma-Aldrich.

Abbreviations. AG, 1-amino-2-hydroxyguanidine, p-toluenesulfate; CoCl₂, cobalt chloride; CRAMP, cathelicidin-related antimicrobial peptide; fMLP, N-formyl-methionyl-leucyl-phenylalanine; GAS, group A Streptococcus; HIF-1α, hypoxia-inducible factor 1, α-subunit; HRE, hypoxic response element; L-Mim, L-mimosine; MRSA, methicillin-resistant Staphylococcus aureus; NE, neutrophil elastase; OH-pyridone, 3-hydroxy-1,2-dimethyl-4(1H)-pyridone; THB, Todd-Hewitt broth; vHL, von Hippel-Lindau tumor-suppressor protein.

### REFERENCES

1. Vogelberg, K. H., and Konig, M. 1993. Hypoxia of diabetic feet with abnormal arterial blood flow. Clin. Investig. 71:466-470.
2. Arnold, F., West, D., and Kumar, S. 1987. Wound healing: the effect of macrophage and tumour derived angiogenesis factors on skin graft vascularization. Br. J. Exp. Pathol. 68:569-574.
3. Negus, R. P., Stamp, G. W., Hadley, J., and Balkwill, F. R. 1997. Quantitative assessment of the leukocyte infiltrate in ovarian cancer and its relationship to the expression of C—C chemokines. Am. J. Pathol. 150:1723-1734.
4. Semenza, G. L. 2001. HIF-1 and mechanisms of hypoxia sensing. Curr. Opin. Cell Biol. 13:167-171.
5. Flamme, I. et al. 1997. HRF, a putative basic helix-loop-helix-PAS-domain transcription factor is closely related to hypoxia-inducible factor-1α and developmentally expressed in blood vessels. Mech. Dev. 63:51-60.
6. Ema, M. et al. 1997. A novel bHLH-PAS factor with close sequence similarity to hypoxia-inducible factor-1α regulates the VEGF expression and is potentially involved in lung and vascular development. Proc. Natl. Acad. Sci. U.S. A. 94:4273-4278.
7. Tian, H., McKnight, S. L., and Russell, D. W. 1997. Endothelial PAS domain protein 1 (EPAS1), a transcription factor selectively expressed in endothelial cells. Genes Dev. 11:72-82.
8. Gu, Y. Z., Moran, S. M., Hogenesch, J. B., Wartman, L., and Bradfield, C. A. 1998. Molecular characterization and chromosomal localization of a third α-class hypoxia inducible factor subunit, HIF3α. Gene Expr. 7:205-213.
9. Burke, B. et al. 2002. Expression of HIF-1α by human macrophages: implications for the use of macrophages in hypoxia-regulated cancer gene therapy. J. Pathol. 196:204-212.
10. Hollander, A. P., Corke, K. P., Freemont, A. J., and Lewis, C. E. 2001. Expression of hypoxia-inducible factor 1α by macrophages in the rheumatoid synovium: implications for targeting of therapeutic genes to the inflamed joint. Arthritis Rheum. 44:1540-1544.
11. Cramer, T. et al. 2003. HIF-1α is essential for myeloid cell-mediated inflammation. Cell. 112:645-657.
12. Schwartz, R. H., Gerber, M. A., and McCoy, P. 1985. Effect of atmosphere of incubation on the isolation of group A streptococci from throat cultures. J. Lab. Clin. Med. 106:88-92.
13. Warnecke, C. et al. 2003. Activation of the hypoxia-inducible factor-pathway and stimulation of angiogenesis by application of prolyl hydroxylase inhibitors. FASEB J 17:1186-1188.
14. Kong, T., Eltzschig, H. K., Karhausen, J., Colgan, S. P., and Shelley, C. S. 2004. Leukocyte adhesion during hypoxia is mediated by HIF-1-dependent induction of 132 integrin gene expression. Proc. Natl. Acad. Sci. U.S.A. 101:10440-10445.
15. Reeves, E. P. et al. 2002. Killing activity of neutrophils is mediated through activation of proteases by K+ flux. Nature. 416:291-297.
16. Guyonnet, V., Johnson, J. K., Bangalore, N., Travis, J., and Long, P. L. 1991. In vitro activity of the human neutrophil cathepsin G on Eimeria tenella sporozoites. J. Parasitol. 77:775-779.
17. Miyasaki, K. T., and Bodeau, A. L. 1991. In vitro killing of oral Capnocytophaga by granule fractions of human neutrophils is associated with cathepsin G activity. J. Clin. Invest. 87:1585-1593.
18. Shafer, W. M., and Onunka, V. C. 1989. Mechanism of staphylococcal resistance to non-oxidative antimicrobial action of neutrophils: importance of pH and ionic strength in determining the bactericidal action of cathepsin G. J. Gen. Microbiol. 135:825-830.
19. Barrett, A. J. 1981. Cathepsin G. Methods Enzymol. 80:561-565.
20. Cole, A. M. et al. 2001. Inhibition of neutrophil elastase prevents cathelicidin activation and impairs clearance of bacteria from wounds. Blood. 97:297-304.
21. Sorensen, O. E. et al. 2001. Human cathelicidin, hCAP-18, is processed to the antimicrobial peptide LL-37 by extracellular cleavage with proteinase 3. Blood. 97:3951-3959.
22. Nizet, V., and Gallo, R. L. 2003. Cathelicidins and innate defense against invasive bacterial infection. Scand. J. Infect. Dis. 35:670-676.
23. Panyutich, A., Shi, J., Boutz, P. L., Zhao, C., and Ganz, T. 1997. Porcine polymorphonuclear leukocytes generate extracellular microbicidal activity by elastase-mediated activation of secreted proprotegrins. Infect. Immun. 65:978-985.
24. Nizet, V. et al. 2001. Innate antimicrobial peptide protects the skin from invasive bacterial infection. Nature. 414:454-457.
25. Lirk, P., Hoffmann, G., and Rieder, J. 2002. Inducible nitric oxide synthase-time for reappraisal. Curr. Drug Targets Inflamm. Allergy. 1:89-108.
26. Gyurko, R. et al. 2003. Mice lacking inducible nitric oxide synthase demonstrate impaired killing of Porphyromonas gingivalis. Infect. Immun. 71:4917-4924.
27. Alam, M. S. et al. 2002. Role of nitric oxide in host defense in murine salmonellosis as a function of its antibacterial and antiapoptotic activities. Infect. Immun. 70:3130-3142.
28. Melillo, G. et al. 1997. Functional requirement of the hypoxia-responsive element in the activation of the inducible nitric oxide synthase promoter by the iron chelator desferrioxamine. J. Biol. Chem. 272:12236-12243.
29. Jung, F., Palmer, L. A., Zhou, N., and Johns, R. A. 2000. Hypoxic regulation of inducible nitric oxide synthase via hypoxia inducible factor-1 in cardiac myocytes. Circ. Res. 86:319-325.
30. Hu, R., Dai, A., and Tan, S. 2002. Hypoxia-inducible factor 1α upregulates the expression of inducible nitric oxide synthase gene in pulmonary arteries of hypoxic rat. Chin. Med. J. 115:1833-1837.
31. Thornton, R. D. et al. 2000. Interleukin 1 induces hypoxia-inducible factor 1 in human gingival and synovial fibroblasts. Biochem. J. 350:307-312.
32. Hellwig-Burgel, T., Rutkowski, K., Metzen, E., Fandrey, J., and Jelkmann, W. 1999. Interleukin-113 and tumor necrosis factor-α stimulate DNA binding of hypoxia-inducible factor-1. Blood. 94:1561-1567.
33. Sandau, K. B., Fandrey, J., and Brune, B. 2001. Accumulation of HIF-1α under the influence of nitric oxide. Blood. 97:1009-1015.
34. Zhou, J., Fandrey, J., Schumann, J., Tiegs, G., and Brune, B. 2003. NO and TNF-alpha released from activated macrophages stabilize HIF-1 alpha in resting tubular LLC-PK1 cells. Am. J. Physiol. Cell Physiol. 284:C439-C446.
35. Zhou, J., Schmid, T., and Brune, B. 2003. Tumor necrosis factor-alpha causes accumulation of a ubiquitinated form of hypoxia inducible factor-1 through a nuclear factor-kappaB-dependent pathway. Mol. Biol. Cell. 14:2216-2225.
36. Kasuno, K. et al. 2004. Nitric oxide induces hypoxia-inducible factor 1 activation that is dependent on MAPK and phosphatidylinositol 3-kinase signaling. J. Biol. Chem. 279:2550-2558.
37. Chan, A. T., Cleeve, V., and Daymond, T. J. 2002. Necrotising fasciitis in a patient receiving infliximab for rheumatoid arthritis. Postgrad. Med. J. 78:47-48.
38. Nemoto, Y. et al. 1999. Differential effects of interleukin-4 and interleukin-10 on nitric oxide production by murine macrophages. Inflamm. Res. 48:643-650.
39. Drapier, J. C., Wietzerbin, J., and Hibbs, J. B. Jr. 1988. Interferon-gamma and tumor necrosis factor induce the L-arginine-dependent cytotoxic effector mechanism in murine macrophages. Eur. J. Immunol. 18:1587-1592.
40. Corradin, S. B. et al 1993. Induction of macrophage nitric oxide production by interferon-gamma and tumor necrosis factor-alpha is enhanced by interleukin-10. Eur. J. Immunol. 23:2045-2048.
41. Eigler, A., Moeller, J., and Endres, S. 1995. Exogenous and endogenous nitric oxide attenuates tumor necrosis factor synthesis in the murine macrophage cell line RAW 264.7. J. Immunol. 154:4048-4054.
42. Marcinkiewicz, J., Grabowska, A., and Chain, B. 1995. Nitric oxide up-regulates the release of inflammatory mediators by mouse macrophages. Eur. J. Immunol. 25:947-951.
43. Kuo, H. P. et al. 2000. Nitric oxide modulates interleukin-1beta and tumor necrosis factor-alpha synthesis by alveolar macrophages in pulmonary tuberculosis. Am. J. Respir. Crit. Care Med. 161:192-199.
44. Iuvone, T., D'Acquisto, F., Carnuccio, R., and Di Rosa, M. 1996. Nitric oxide inhibits LPS-induced tumor necrosis factor synthesis in vitro and in vivo. Life Sci. 59:PL207-PL211.
45. Wu, C. H. et al. 2003. Nitric oxide modulates pro- and anti-inflammatory cytokines in lipopolysaccharide-activated macrophages. J. Trauma. 55:540-545.
46. Griffiths, L. et al. 2000. The macrophage—a novel system to deliver gene therapy to pathological hypoxia. Gene Ther. 7:255-262.
47. Talks, K. L. et al. 2000. The expression and distribution of the hypoxia-inducible factors HIF-1α and HIF-2α in normal human tissues, cancers, and tumor-associated macrophages. Am. J. Pathol. 157:411-421.
48. Nathan, C. 2003. Immunology: oxygen and the inflammatory cell. Nature. 422:675-676.
49. Strieter, R. M. 2003. Mastering innate immunity. Nat. Med. 9:512-513.
50. Clausen, B. E., Burkhardt, C., Reith, W., Renkawitz, R., and Forster, I. 1999. Conditional gene targeting in macrophages and granulocytes using LysMcre mice. Transgenic Res. 8:265-277.
51. Walmsley, S. R. et al. 2005. Hypoxia-induced neutrophil survival is mediated by HIF-1α-dependent NF-B activity. J. Exp. Med. 201:105-115.
52. Kansal, R. G., McGeer, A., Low, D. E., Norrby-Teglund, A., and Kotb, M. 2000. Inverse relation between disease severity and expression of the streptococcal cysteine protease, SpeB, among clonal M1T1 isolates recovered from invasive group A streptococcal infection cases. Infect. Immun. 68:6362-6369.
53. Liu, G. Y. et al. 2004. Sword and shield: linked group B streptococcal β-hemolysin/cytolysin and carotenoid pigment function to subvert host phagocyte defense. Proc. Natl. Acad. Sci. U.S.A. 101:14491-14496.
54. Humar, D. et al. 2002. Streptolysin S and necrotising infections produced by group G streptococcus. Lancet. 359:124-129.
55. Grunstein, J., Roberts, W. G., Mathieu-Costello, O., Hanahan, D., and Johnson, R. S. 1999. Tumor-derived expression of vascular endothelial growth factor is a critical factor in tumor expansion and vascular function. Cancer Res. 59:1592-1598.
56. Blouw, B. et al. 2003. The hypoxic response of tumors is dependent on their microenvironment. Cancer Cell. 4:133-146.

1-7. (canceled)

8. A method for the prophylaxis and/or treatment of infection or virulence in a subject in need thereof, comprising administering to said subject a pharmaceutically effective amount of a HIF-1 modulating compound.

9. The method of claim 8, wherein said HIF-1 modulating compound is a HIF-1 agonist.

10. The method of claim 9, wherein said agonist is a compound that stabilizes HIF-1.

11. The method of claim 8, wherein said compound is a hydroxylase inhibitor.

12. The method of claim 8, wherein said HIF-1 modulating compound is a substrate-based inhibitor.

13. The method of claim 11, wherein said substrate-based inhibitor is 3-exomethyleneproline peptide like compounds.

14. The method of claim 11, wherein said substrate-based inhibitor is a proline derivative.

15. The method of claim 11, wherein said substrate-based inhibitor is a 4(S)hydroxy proline derivative.

16. The method of claim 11, wherein said substrate-based inhibitor is a 4-keto proline derivative.

17. The method of claim 8, wherein said HIF-1 modulating compound is a cofactor-based inhibitor.

18. The method of claim 8, wherein said HIF-1 modulating compound is a 2-oxoglutarate analogue, ascorbic acid analogue or an iron chelator.

19. The method of claim 18, wherein said compound is mimosine or a mimosine analog.

20. The method of claim 8, wherein HIF-1 modulating compound is a compound which stabilizes HIF-1α under normoxic conditions.

21. The method of claim 8, wherein said subject has an infection, a symptom of an infection, or a predisposition toward an infection.

22. The method of claim 8, wherein said infection or virulence is pathogenic.

23. The method of claim 8, wherein the infection is a bacterial, protozoal, fungal, nematode, or viral infection.

24. The method of claim 8, wherein the infection is by a pathogen that is antibiotic resistant.

25. The method of claim 8 further comprising administering to said subject a pharmaceutically effective amount of an antibiotic.

26. The method of claim 8 further comprising administering to said subject a pharmaceutically effective amount of an anti-fungal.

27. The method of claim 8 further comprising administering to said subject a pharmaceutically effective amount of an anti-viral.

28. The method of claim 8 further comprising administering to said subject a pharmaceutically effective amount of an anti-inflammatory.

29. The method of claim 8 further comprising administering to said subject a vaccine formulation of a pathogen.

30. A method for increasing the killing capacity of the cells of the innate immune system in a subject, said method comprising administering to said subject a pharmaceutically effective amount of a HIF-1 modulating compound.

31-32. (canceled)

33. A method for screening compounds for inhibiting infection or disease in a subject, or which induce or stimulate a host's pathogenic defense mechanisms, said method comprising:
screening compounds that increase or maintain the activity or level of HIF-1, and
identifying compounds that increase or facilitate the ability of a cell of the innate immune response to inhibit or reduce pathogen infectivity or virulence.

34-44. (canceled)

45. A method of prevention of respiratory tract infections in a subject in need thereof, comprising administering to said subject a pharmaceutically effective amount of a HIF-1 modulating compound.

46. A method of prevention of transmissible diseases in a subject in need thereof, comprising administering to said subject a pharmaceutically effective amount of a HIF-1 modulating compound.

47. (canceled)
